(19) United States
(12) Patent Application Publication
(10) Pub. No.: US 2005/0166269 A1
(43) Pub. Date: Jul. 28, 2005

(54) Sleep genes in Drosophila and their use for the screening, diagnosis and therapy of sleep disorders

(76) Inventors: Giulio Tononi, Verona, WI (US); Chiara Cirelli, Verona, WI (US)

Correspondence Address: Steven L. Highlander;Fulbright & Jaworski L.L.P., 600 Congress Avenue, Suite 2400, Austin, TX, 78701, US

(21) Appl. No.: 11/013,314

(22) Filed: Dec. 15, 2004

Related U.S. Application Data
(60) Provisional application No. 60/529,536, filed on Dec. 15, 2003.
(60) Provisional application No. 60/563,858, filed on Apr. 20, 2004.

(51) Int. Cl. A01K 67/033

(52) U.S. Cl. 800/3; 435/4; 800/8

(57) ABSTRACT

Methods of screening for a sleep altering compositions are disclosed as are the identities of various gene products that are involved in sleep function/dysfunction. Also described are methods for modifying the need for sleep and the response to sleep deprivation in subjects.

This application claims benefit of priority to U.S. Provisional Application Ser. No. 60/529,536, filed Dec. 15, 2003 and U.S. Provisional Application Ser. No. 60/563,858, filed Apr. 20, 2004, the entire contents of which are hereby incorporated by reference.

This invention was made with United States government support awarded by the following agencies: the Department of the Army and MRMC (DAAD 19-02-1-0036). The United States has certain rights in this invention.

### BACKGROUND OF THE INVENTION

1. Field of the Invention

The present invention relates generally to the fields of molecular biology, cell biology, and pharmacology. More particularly, it concerns the identification of sleep-related genes in Drosophila, and methods of screening for sleep-altering compositions that affect the expression or activity of these genes. The present invention also pertains to methods of modifying the need for sleep and the response to sleep deprivation, as well as methods of identifying the basis of a sleep disorder in a subject.

2. Description of Related Art

Sleep is a state of reduced consciousness in which the brain is relatively more responsive to internal than to external stimuli. Normal sleep is divided into non-rapid eye movement (NREM) and rapid eye movement (REM) sleep. In humans, the stages of sleep are stage I (light sleep), stage II, stages III and IV (deep or delta-wave sleep), and REM sleep. NREM sleep comprises stages I-IV.

The most prominent effect of total sleep deprivation in humans is cognitive impairment, with striking practical consequences. Each year, errors due to sleep deprivation and sleepiness cause 25,000 deaths, 2.5 millions of disabling injuries, and cost over $56,000,000,000 in the U.S. alone (National Commission on Sleep Disorders Research, 1994). Moreover, the National Highway Traffic Safety Administration estimates conservatively that each year drowsy driving is responsible for at least 100,000 automobile crashes, 71,000 injuries, and 1,550 fatalities (National Sleep Foundation, 2002). A sleep-deprived person tends to take longer to respond to stimuli, particularly when tasks are monotonous and low in cognitive demands. However, sleep deprivation produces more than just decreased alertness. Tasks emphasizing higher cognitive functions, such as logical reasoning, encoding, decoding and parsing complex sentences, complex subtraction tasks and tasks requiring divergent thinking, such as those involving a flexible thinking style and the ability to focus on a large number of goals simultaneously, are all significantly affected even after one single night of sleep deprivation. Tasks requiring sustained attention, such as those including goal-directed activities, can also be impaired by even a few hours of sleep loss.

NREM sleep is controlled by complex initiating and maintenance mechanisms, the extent of which is not fully known (reviewed in Saper et al., 2001; Belenky et al., 2003; Pace-Schott and Hobson, 2002). Probably no single sleep generating center exists. A more likely mechanism is sleep-generating circuits with inputs from the brainstem and hypothalamic neuronal groups. REM sleep is generated by mesencepthalic and pontine cholinergic neurons. It is characterized by muscle atonia, cortical activation, low-voltage desynchronization of the EEG, and rapid eye movements. REM has both tonic and phasic characteristics. Tonic muscle atonia is present throughout REM sleep. It results from inhibition of alpha motor neurons by clusters of peri-locus ceruleus neurons, which are referred to collectively as the dorsolateral small cell reticular group.

As the function of sleep has not been fully determined, the absolute number of hours necessary to fulfill its function in humans is still unknown. Some individuals claim full effectiveness with only 3-5 hours of sleep per night, while some admit needing at least 8 hours of sleep per night or more to perform effectively. Sleep deprivation is best defined by group means and in terms of the tasks impaired.

With decreased sleep, higher-order cognitive tasks are affected early and disproportionately (Belenky et al., 2003; Van Dongen et al., 2003). Tests requiring both speed and accuracy demonstrate considerably slowed speed before accuracy begins to fail. Total sleep duration of 7 hours per night over 1 week has resulted in decreased speed in tasks of both simple reaction time and more demanding computer-generated mathematical problem solving. Total sleep duration of 5 hours per night over 1 week shows both decrease in speed and the beginning of accuracy failure. Total sleep duration of 7 hours per night over 1 week leads to impairment of cognitive work requiring simultaneous focus on several tasks.

Sleep loss causes attention deficits, decrease in short-term memory, speech impediments, perseveration and inflexible thinking. These deficits can explain why sleep deprived subjects underestimate the severity of their cognitive impairment, often with tragic consequences. Another reason is the fact that the lack of sleep does not completely eliminate the capacity to perform, but rather makes the performance inconsistent and unreliable. Thus, a sleepy driver will either respond normally to an emergency or not at all, due to rapid changes in vigilance state and the sudden intrusion of “microsleeps,” defined as brief runs of theta or delta activities that break through the otherwise beta or alpha EEG of waking, during waking. Similarly, subjects may still be able to transiently perform at baseline levels in short tests even after 3-4 days of sleep deprivation. However, the same subjects will perform very poorly when engaged in tasks requiring sustained attention. New evidence suggests that not just a few hours of sleep, but several days of normal sleep/waking patterns are required to normalize cognitive performance after sleep deprivation.

Sleep deprivation is a relative concept. Small amounts of sleep loss (e.g., 1 hour per night over many nights) have subtle cognitive costs, which appear to go unrecognized by the individual experiencing the sleep loss (Belenky et al., 2003; Van Dongen et al., 2003). More severe restriction of sleep for a week leads to profound cognitive deficits similar to those seen in some stroke patients, which also appear to go unrecognized by the individual. The lack of recognition of the effects of sleep deprivation are not uncommon.

Chronic disease is also associated with sleep disorders. For example, problems like stroke and asthma attacks tend to occur more frequently during the night and early morning, perhaps due to changes in hormones, heart rate, and other characteristics associated with sleep. Sleep also affects some kinds of epilepsy in complex ways. REM sleep seems to help prevent seizures that begin in one part of the brain from spreading to other brain regions, while deep sleep may promote the spread of these seizures. Sleep deprivation also triggers seizures in people with some types of epilepsy.

Sleeping problems occur in almost all people with mental disorders, including those with depression and schizophrenia. People with depression, for example, often awaken in the early hours of the morning and find themselves unable to get back to sleep. The amount of sleep a person gets also strongly influences the symptoms of mental disorders. Sleep deprivation is an effective therapy for people with certain types of depression, while it can actually cause depression in other people. Extreme sleep deprivation can lead to a seemingly psychotic state of paranoia and hallucinations in otherwise healthy people, and disrupted sleep can trigger episodes of mania (agitation and hyperactivity) in people with manic depression. Sleeping problems are common in many other disorders as well, including Alzheimer's Disease, stroke, cancer, and head injury. These sleeping problems may arise from changes in the brain regions and neurotransmitters that control sleep, or from the drugs used to control symptoms of other disorders.

A greater understanding of the factors that affect sleep would facilitate the development of new and improved treatments of sleep disorders and sleep deprivation. Knowledge of such factors may result in the development of compounds to assist in continuous performance or sleep deprivation recovery, and would be particularly valuable in many branches of military, airline, medical and emergency, and security industries.

### SUMMARY OF THE INVENTION

Thus, in accordance with the present invention, there is provided a method of screening for a sleep altering composition comprising (a) providing a Drosophila cell; (b) contacting said cell with a candidate compound; and (c) measuring the effect of said compound on expression level or activity of a first gene product encoded by the group of genes consisting of CG18190, Jheh 1, CG7228, lama, disco, CG6664, Casein kinase II β subunit, CG9171, GstE1, cAMP-dependent protein kinase R2, CG15161, MESR3, Meics, Atpalpha, Calx, Rlip, nompC, H15, Lam, Glu-RIIA, Glu-RIIB, Ork1, Shaker, and Hyperkinetic, whereby a change in the expression level or activity of said gene product, as compared to the expression level or activity of said gene product in a similar cell not treated with said candidate compound, indicates that said candidate compound is a sleep altering composition. The Drosophila cell may be a neuronal cell. The cell may be located in a living fly. The composition may promote sleep, inhibit sleep, promote recovery from sleep deprivation or reduce the need for sleep.

The method may further comprise measuring the effect of said compound on the expression level or activity of a second gene product from said group. Measuring expression level may comprises measuring mRNA levels for said first gene product, measuring mRNA turnover for said first gene product, measuring protein levels for said first gene product. Measuring may further comprise a technique selected from the group consisting of quantitative RT-PCR Northern blot, ELISA or Western blot. Measuring activity may also comprise an assay for enzyme function or binding function. The method may also further comprise measuring the expression level or activity of said gene product in a similar cell not treated with said candidate compound, i.e., a negative control. The method may also further comprise treating said cell with a known sleep modulating composition, i.e., a positive control. The method may further comprise assessing the effect of said candidate substance on an intact organism.

In another embodiment, there is provided a method of reducing the need for sleep in a subject comprising modulating the expression level or activity of a gene product encoded by the group consisting of CG18190, Jheh 1, CG7228, lama, disco, CG6664, Casein kinase II β subunit, CG9171, GstE1, cAMP-dependent protein kinase R2, CG15161, MESR3, Meics, Atpalpha, Calx, Rlip, nompC, H15, Lam, Glu-RIIA, Glu-RIIB, Ork1, and Shaker, and Hyperkinetic. The expression level or activity of one or more gene product encoded by CG18190 and Jheh 1 may be increased, for example, by providing the gene product or small molecule agonist to said subject. The gene product or agonist may be provided to said subject multiple times over a defined period. The method may also further comprise providing a stimulant to said subject. Alternatively, the expression level or activity of Ork1 may be decreased, for example, by providing an antisense molecule, a ribozyme, an interfering RNA or an antagonist small molecule to said subject. The antisense, ribozyme, siRNA or antagonist may be provided to said subject multiple times over a defined period. The subject may suffer from a sleep disorder or from environmental sleep deprivation.

Yet another embodiment comprises a method of promoting recovery from sleep loss in a subject comprising modulating the expression level or activity of a gene product encoded by from the group of genes consisting of CG18190, Jheh 1, CG7228, lama, disco, CG6664, Casein kinase II β subunit, CG9171, GstE1, cAMP-dependent protein kinase R2, CG15161, MESR3, Meics, Atpalpha, Calx, Rlip, nompC, H15, Lam, Glu-RIIA, Glu-RIIB, Ork1, and Shaker, and Hyperkinetic. The expression level or activity of one or more gene products encoded by CG18190 or Jheh 1 may be increased, for example, by providing the gene product or an agonist small molecule to said subject. The gene product or agonist may be provided to said subject multiple times over a defined period. The method may further comprise providing a stimulant to said subject. Alternatively, the expression level or activity of Ork1 may be decreased, for example, by providing an antisense molecule, a ribozyme, an interfering RNA or an antagonist small molecule to said subject. The antisense molecule, ribozyme, interfering RNA or antagonist small molecule may be provided to said subject multiple times over a defined period. The subject may suffer from a sleep disorder or from environmental sleep deprivation.

In still yet another embodiment, there is provided a method of inhibiting sleep in a subject comprising modulating the expression level or activity of a gene product encoded by a gene selected from the group consisting of CG18190, Jheh 1, CG7228, lama, disco, CG6664, Casein kinase II β subunit, CG9171, GstE1, cAMP-dependent protein kinase R2, CG15161, MESR3, Meics, Atpalpha, Calx, Rlip, nompC, H15, Lam, Glu-RIIA, Glu-RIIB, Ork1, and Shaker, and Hyperkinetic. The expression level or activity of one or more gene product encoded by CG18190 or Jheh 1 may be increased, for example, by providing the gene product or an agonist small molecule to said subject. The gene product or agonist may be provided to said subject multiple times over a defined period. The method may further comprise providing a stimulant to said subject. Alternatively, the expression level or activity of Ork1 may be decreased, for example, by providing an antisense molecule, a ribozyme, an interfering RNA or an antagonist small molecule to said subject. The antisense molecule, ribozyme, interfering RNA or antagonist small molecule may be provided to said subject multiple times over a defined period. The subject may suffer from a sleep disorder or from environmental sleep deprivation.

In a further embodiment, there is provided a method of increasing sleep in a subject comprising modulating the expression level or activity of a gene product encoded by the group consisting CG18190, Jheh 1, CG7228, lama, disco, CG6664, Casein kinase II β subunit, CG9171, GstE1, cAMP-dependent protein kinase R2 , CG15161, MESR3, Meics, Atpalpha, Calx, Rlip, nompC, H15, Lam, Glu-RIIA, Glu-RIIB, Ork1, and Shaker, and Hyperkinetic. The expression level or activity of Ork1 may be increased, for example, by providing the gene product or an agonist small molecule to said subject. The gene product or agonist may be provided to said subject multiple times over a defined period. The method may further comprise providing a sedative to said subject. Alternatively, the expression level or activity of one or more gene product encoded by CG18190 or Jheh 1 may be decreased, for example, by providing an antisense molecule, a ribozyme, an interfering RNA or an antagonist small molecule to said subject. The antisense molecule, ribozyme, interfering RNA or antagonist small molecule may be provided to said subject multiple times over a defined period. The subject may suffer from a sleep disorder.

In still yet a further embodiment, there is provided a method for identifying the basis of a sleep disorder in a subject comprising (a) obtaining mRNA from a neuronal cell of said subject; and (b) measuring the expression level or activity of SEQ ID NOS:2, 4, 6, 8, 10, 12, 14, 16, 18, 20, 22, 24, 26, 28-30, 32, 34, 36, 38, 40, 42, 44, 46, 48, 54 and/or 55, whereby a change in the expression level or activity of a gene product in step (b), as compared to the expression level or activity of said gene product in a similar cell from a normal subject, identifies the basis of said sleep disorder.

Other embodiments include an isolated and purified nucleic acid comprising a segment encoding a polypeptide selected from the group consisting of SEQ ID NOS:2, 4, 6, 8, 10, 12, 14, 16, 18, 20, 22, 24, 26, 28-30, 32, 34, 36, 38, 40, 42, 44, 46, 48, 54 and/or 55. The nucleic acid may further comprise a promoter operably linked to said segment, wherein said promoter is active in eukaryotic cells, an may also further comprise a replication competent vector. The vector may be a plasmid vector or a viral vector. The nucleic acid may comprise a DNA sequence selected from the group consisting of CG18190, Jheh 1, CG7228, lama, disco, CG6664, Casein kinase II β subunit, CG9171, GstE1, cAMP-dependent protein kinase R2, CG15161, MESR3, Meics, Atpalpha, Calx, Rlip, nompC, H15, Lam, Glu-RIIA, Glu-RIIB, Ork1, Shaker, and Hyperkinetic.

The present invention further encompasses an isolated and purified polypeptide selected from the group consisting of SEQ ID NOS:2, 4, 6, 8, 10, 12, 14, 16, 18, 20, 22, 24, 26, 28-30, 32, 34, 36, 38, 40, 42, 44, 46, 48, 54 and/or 55. In addition, an isolated and purified peptide of no more than about 50 amino acids in length comprising a segment of 15 or more consecutive residues from a polypeptide selected from the group consisting of SEQ ID NOS:2, 4, 6, 8, 10, 12, 14, 16, 18, 20, 22, 24, 26, 28-30, 32, 34, 36, 38, 40, 42, 44, 46, 48, 54 and/or 55 is contemplated. The segment may comprise 20 or more, 25 or more, 30 or more, 35 or more, 40 or more, 45 or more, 50 or more 75 or more, or up to about 100 consecutive residues of said polypeptide.

Also provided are an isolated and purified oligonucleotide of no more than about 50 nucleotides in length comprising a segment of 15 or more consecutive bases from a polynucleotide selected from the group consisting of SEQ ID NOS:1, 3, 5, 7, 9, 11, 13, 15, 17, 19, 21, 23, 25, 27, 31, 33, 35, 37, 39, 41, 43, 45, 47 and/or 53, such as where the segment comprises 20 or more, 25 or more, 30 or more, 35 or more, 40 or more, 45 or more, 50 or more, 75 or more or about 100 consecutive bases of said polynucleotide. The oligonucleotide may be labeled with a detectable label.

Polyclonal antisera, antibodies of which bind immunologically to a polypeptide selected from the group consisting of SEQ ID NOS:2, 4, 6, 8, 10, 12, 14, 16, 18, 20, 22, 24, 26, 28-30, 32, 34, 36, 38, 40, 42, 44, 46, 48, 54 and/or 55, or a monoclonal antibody that binds immunologically to a polypeptide selected from the group consisting of SEQ ID NOS:2, 4, 6, 8, 10, 12, 14, 16, 18, 20, 22, 24, 26, 28-30, 32, 34, 36, 38, 40, 42, 44, 46, 48, 54 and/or 55, are provided as well.

As used herein the specification, “a” or “an” may mean one or more. As used herein in the claim(s), when used in conjunction with the word “comprising”, the words “a” or “an” may mean one or more than one. As used herein “another” may mean at least a second or more.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-D. Analysis of locomotor activity and sleep in fruit flies.

FIG. 1A—Schematic of the ultrasound activity monitoring system. A 44-kHz standing wave is passed across an independent enclosure containing a single fly. An integrated circuit samples a portion of each wave as a function of the transmit signal and compares it to the output from the receive signal for the same time window. When the fly moves its mass within the field, it perturbs the standing wave, and the resulting difference is counted as a movement. The output is sampled by a PC at 200 Hz, data are summed in 2-s bins, and stored for later processing (modified from ref. 23)

FIG. 1B—A Drosophila Activity Monitoring System (DAMS) monitor containing thirty-two 6.5-mm (5 mm I.D.) glass tubes, each housing a single fly.

FIG. 1C—Twenty-four hour locomotor activity of a single female wild-type Canton-S fly as measured by the DAMS infrared system. The fly is mostly active during the light period (from 8 am to 8 pm), and inactive during the dark period, when episodes of uninterrupted quiescence can last for several hours.

FIG. 1D—Typical pattern of sleep in a population of 96 female wild-type Canton-S flies as measured in a DAMS monitor. DAMS measures activity as counts (number of crossings) per minute. Wakefulness is defined as any period of at least 1 minute characterized by activity (one or more counts per minute; see FIG. 1C). Based on arousal threshold data, sleep is defined as any period of uninterrupted behavioral quiescence (no counts/min) lasting for at least 5 min. Mean values of the amount of sleep are calculated on consecutive 30-min time intervals and the time course is graphically shown over the entire day. In female flies, most of the sleep occurs at night (FIGS. 1C-D, Cirelli et al., unpublished data).

FIGS. 2A-D. The homeostatic regulation of sleep in the fruit flies.

FIG. 2A—The sleep deprivation apparatus in the inventors' laboratory. Each of the five framed boxes holds 10 DAMS monitors.

FIG. 2B—Increase in sleep duration following 6, 12, and 24 hours of sleep deprivation (SD) in female Canton-S flies (n=20-40 for each experiment). Each diagram shows the daily amount of sleep for baseline day (blue line), SD day (red line), and the first recovery day after SD (green line). Time and duration of SD are indicated by the red bars below the x axis. An increase in sleep duration is present after all 3 periods of SD, and occurs mainly during the first 6 hours following the end of SD. Flies were maintained in a 12:12 light dark cycle (light on at 8 am).

FIG. 2C—Amount of sleep lost (during SD) and of sleep recovered (during the first 6 hours of recovery day 1) for the experiments shown in FIG. 2B. More sleep is recovered after 12-24 h SD than after 6 h SD. Since female flies sleep mostly during the night, there is no significant difference in the amount of sleep lost and sleep recovered between 24 h SD and 12 h SD during the night. When female flies are sleep deprived for 12 hours during the light period (the corresponding graph is not shown in FIG. 2B), there is no significant sleep loss, nor significant sleep rebound. Note that in flies, as in mammals, the amount of sleep recovered after SD represents only a fraction of the sleep lost.

FIG. 2D—To measure sleep fragmentation, a sleep continuity score is calculated, which increases during continuous epochs with no locomotor activity and decreases during epochs with one or more counts of activity. The sleep continuity score is high if sleep is continuous and undisturbed, and low if sleep is fragmented. Blue lines in the upper diagram represents sleep scores for 16 female Canton-S flies during baseline. Green lines in the lower diagram show the sleep score for the same flies the day following 24 h SD. Note the significant increase in the sleep score immediately after the end of SD. In several flies this increase persists during the following night (FIGS. 2B-D, Cirelli et al., unpublished data).

FIG. 3—The escape response to a complex stimulus in flies. During the vigilance test, flies remain in the DAMS monitor where their locomotor activity is continuously recorded. The stimulus is delivered randomly every 2-10 min at either side of the glass tubes.

FIG. 4—Testing vigilance in wild-type flies before and after sleep deprivation. Wild-type Canton-S flies (n=20) were tested during the first 3 hours of the light period the day before and the day after 24 h of sleep deprivation (SD). During baseline the latency to beam crossing decreases significantly after the stimulus compared to before the stimulus (* P<0.01, paired t-test). After SD, however, the latency to beam crossing is as high before the stimulus as after the stimulus. Thus, even when awake, wild-type flies sleep deprived for 24 hours are impaired in their ability to respond to the stimulus. Similar data wee obtained in white ¹¹¹⁸ flies.

FIGS. 5A-B. Testing memory in flies: the heat box.

FIG. 5A—The heat-box in the inventors' laboratory.

FIG. 5B—A schematic diagram of the apparatus with 3 of the 16 parallel chambers shown (from Zars et al., 2000). A computer receives position information for individual flies from a light gate array. This is used to calculate the performance index (PI). PI is the time spent in the unpunished half of the chamber minus the time spent in the punished half of the chamber, divided by the total time. PI can vary from −1 to +1, with flies that are perfect avoiders having PI=+1. PI=0 indicates no side preference. PI is measured during training, when it is a measure of heat avoidance, and after training, when it is a measure of memory.

FIGS. 6A-C. Identification of short sleeper mutant lines.

FIG. 6A—Intra-individual consistency and inter-individual variability in the daily amount of sleep in fruit flies. Daily amount of sleep is shown for four 7-day old virgin female flies of the same mutant line.

FIG. 6B—Daily amount of sleep in 1547 insertional lines (P lines from ref. 47, female flies). Mean amount of sleep/24 hour is 616±169 (mean±SD; min 131, max 1155). Shaded areas show one (dark red) and two (light red) standard deviations from the mean.

FIG. 6C—Daily amount of sleep in female (upper panel, n=16) and male (lower panel, n=15) flies of a short sleeper line. For comparison, the blue line in each panel represents the daily amount of sleep in wild-type Canton-S flies (n=16).

FIGS. 7A-C—Identification of “no-rebound” mutant lines.

FIG. 7A—Cumulative graph showing the time course of the sleep rebound following 24 hour of sleep deprivation (SD) in female wild-type Canton-S flies. Daily amount of sleep during baseline was 580 min. Sleep recovered is expressed as % of sleep lost. At the end of recovery day 1, ˜40% of sleep was recovered, half of which during the first 2-3 hours following the end of SD (red circle). No further recovery occurred during recovery day 2.

FIG. 7B—Percentage of sleep recovered during the first 6 hours following 24 h SD in 593 insertional lines (P. lines from ref. 47, female flies). Most lines recovered 20% of the sleep lost during SD.

FIG. 7C—Increase in the sleep continuity score after 24 h SD in 593 insertional lines (same lines as in FIG. 7B). Bars indicate sleep scores for the first 6 hours of the light period during baseline (sleep score=37±30, mean±SD, blue bars) and after 24 h SD (118±67, red bars). The higher the sleep score, the lower the sleep fragmentation.

FIGS. 8A-F—Sleep in flies of the lines 1174 and 1179, called ss (short sleepers) flies.

FIG. 8A—Distribution of daily sleep amounts in ˜9000 mutant lines for both female and male flies (16 flies/line, ≧3 independent experiments/line). Shaded areas show one and two standard deviations from the mean (mean±SD, females: 624±167; males: 910±155). Red asterisks indicate ss flies.

FIG. 8B—Daily time course (in 30-min intervals) of the amount of sleep in wild-type Canton-S (CS) and ss flies. Curves connect mean values±SEM (min of sleep/24 hours, CS females (n=63)=664±17; CS males (n=55)=923±21; ss heterozygous females (h, n=50)=564±32; ss homozygous females (ss, n=60)=247±22; ss males (ss, n=58)=297±34). The white and black bars under the x axis indicate the light and dark period, respectively.

FIG. 8C—Arousal threshold differences between epochs of activity and immobility during the dark period. The y axis represents the percentage of escape responses triggered by a complex stimulus of low intensity, which is used as a measure of arousal threshold. Most (≧60%) wild-type (wt) and ss flies respond if they had been active during the minute before the stimulus was delivered (black columns). However, the ability to respond decreases significantly, relative to the periods of activity, when flies are stimulated after a period of immobility of at least 5 min (n=30 flies/line; #, p<0.01, paired t-test). During the dark period the arousal threshold is also significantly decreased after 1 min of immobility. Values are mean±SEM for the entire 12-hour dark period.

FIG. 8D—Duration and number of sleep episodes during 24 hours of baseline recording in wild-type (wt) and ss flies (mean±SEM, 32 flies/line; *, p<0.05, t-test).

FIG. 8E—Daily time course of the amount of sleep in ss flies under 12:12 light-dark conditions (dashed line) and constant darkness (solid line; ss females (n=123)=163±17; ss males (n=34)=283±34).

FIG. 8F—Left panel. Locomotor activity of an individual wild-type fly (upper panel) and ss fly (lower panel) over 6 days in constant darkness. Actograms are double plotted. The grey bar under the plots represents subjective day, the black bar represents subjective night. Right panel. Autocorrelation analysis of locomotor behavior in wild-type and ss female flies kept in constant darkness for 7 consecutive days (n=130 flies/line). The asterisks indicate the rhythmicity index (RI), a measure of the strength of the activity rhythm (RI, wild-type=0.54, ss=0.52). The estimated period is 24.0 hours in wild-type flies and 24.1 hours in ss flies. Autocorrelation analysis for individual ss flies indicated that >90% are rhythmic (data not shown).

FIGS. 9A-D—Response to sleep deprivation (SD) and measures of performance in ss flies.

FIG. 9A—Increase in sleep duration after SD. Black columns represent sleep lost (in min) during 24 hours of SD, grey columns represent sleep gain—the number of minutes flies overslept relative to baseline during the first 24 h after SD (#, p<0.05, paired t-test). The amount of sleep recovered, expressed as percentage of sleep lost (red columns) ranges between 10 and 25% and is similar in wild type (wt, females and males) and ss flies. Note that positive and negative values on the y axis are on different scales.

FIG. 9B—Increase in sleep intensity after SD. In all flies the number of brief awakenings (upper panel) is significantly reduced during the light period after SD relative to baseline, while the duration of the sleep episodes (middle panel) is significantly increased (#, p<0.05, paired t-test, n=32 flies/line). In both cases the change is significantly smaller in ss flies relative to wild-type flies (*, p<0.05, t-test). Lower panel. Arousal threshold was measured as in FIG. 1C. Black columns represent the percentage of escape response in flies that had been moving the minute before the stimulus was delivered, while white and grey columns refer to flies that have been immobile for 5 min (sleeping flies). The percentage of flies responding to the stimulus is lower during recovery sleep after SD (grey columns) relative to baseline sleep (white columns) in both wild-type and ss flies, but the change is significant only in wild-type flies (n=32 flies/line; #, p<0.05; paired t-test) but not in ss flies (O=0.08). Values are mean±SEM for the first 6 hours of the light period after SD.

FIG. 9C—Upper panel. Locomotor activity measured in the Drosophila Activity Monitoring System as activity index (AI)—the number of beam crossings/min (n=16 female (f) and 16 male (m) flies/line, light period). Values were calculated including only awake flies. Lower panel. Locomotor behavior measured in the heat box during the 10-min adaptation period preceding the delivery of the thermal stimulus (all flies were awake during that period). The distance traveled per min is measured in arbitrary units (n=25 flies/line).

FIG. 9D—Assessment of performance before and after SD. Upper panel. The response to a complex stimulus is measured as the percentage increase in the number of beam crossings during the minute following the delivery of the stimulus relative to the minute prior to the stimulation. In wild-type flies, but not in ss flies, the increase is significantly reduced during recovery (rec) after SD relative to baseline (bl). All flies had been active (i.e., awake) during the minute before the delivery of the stimulus. Values are mean±SEM averaged for the entire light period (one stimulus/hour; n=32 flies/line, #, p<0.05, paired t-test). Lower panel. The response to a thermal stimulus is measured as the latency (in sec) to beam crossing after heat was applied to the side of the chamber housing the flies (mean±SEM, n=32 flies/line). The escape response after SD worsens (i.e., latency increases) in wild-type flies but not in ss flies. Each fly was tested once during the first 2 hours after the end of SD and at the corresponding time of day during baseline (#, p<0.05, paired t-test).

FIGS. 10A-D—The Shaker channel and the ss mutation.

FIG. 10A—The alpha subunit of the Shaker channel includes 6 transmembrane segments: S1-S4 form the voltage-sensor module, S5-S6 form the pore region.

FIG. 10B—Schematic representation of the Shaker transcription unit with 19 exons. The grey bar indicates the N-terminal variable region, the green bar indicates the common central region, and the blue bar indicates the C-terminal variable region. The red arrow indicates the approximate location of the ss mutation.

FIG. 10C—Sequence alignment of the S1 domain. The threonine residue is conserved between Shaker homologues in different species.

FIG. 10D—Shaker transcripts from fly heads and bodies. The probe was a fragment of 550 bp spanning exons 9 and 10. To check that equal amounts of RNA were being compared blots were reprobed with probes to actin (not shown).

FIGS. 11A-C—Genetic mapping of the shaking and short sleep phenotype in ss flies.

FIG. 11A—Cytological and genetic locations of the markers used to map the phenotypes.

FIG. 11B—Crossing scheme to generate recombinants. Left, heterozygous females (v f/Sh^{ss}) were crossed to v f males and the male progeny were divided by phenotype (shaking, forked and vermillion) into one of the six genotypes. Right, daily sleep amount for each of the six genotypes. Number (N) indicates the number of individual flies tested. In classes 1 through 4 the male progeny from the cross were directly tested. In classes 5 and 6 (*), there were not enough isolates to generate a statistically valid number. Instead, males in classes 5 and 6 were crossed to females (C(1)x) and the recombinant chromosomes generated from this cross were individually tested. Two recombinant chromosomes in classes 5 and eight recombinant chromosomes in class 6 consistently produced a short sleeper and a normal sleeper phenotype, respectively. As such, the individual data were combined for this graph.

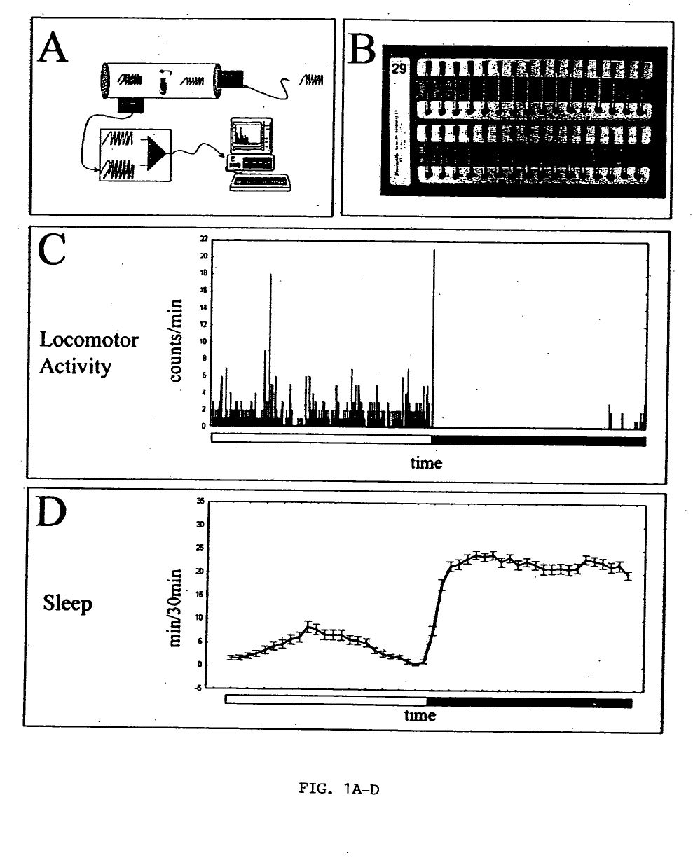

FIG. 1C—Complementation results between Sh^{ss} and previously described Sh alleles for the recessive ss phenotype. The Allele/Sh^{ss} heterozygous females were generated from crosses between Sh^{ss} females and males with the Sh allele indicated in the first column on the left. For comparison, Allele/Sh⁺ heterozygous females are included. The Allele/Sh⁺ were generated by crossing w¹¹¹⁸ females to males with the Sh allele indicated in the first column on the left. Daily sleep amount (mean±SEM, min/24 h) was recorded over two days. Female flies with daily sleep amount≦290 min are below 2 standard deviations from the norm (FIG. 1A). Only Sh¹⁰²/Sh^{ss} shows a strong short sleeper phenotype and therefore fails to complement the ss phenotype of Sh^{ss}.

FIG. 12—Distribution of daily sleep amounts in male flies of ˜9000 mutant lines and in several Shaker alleles (thin black lines). The null alleles Sh¹⁰² Sh¹³³, and Sh^{M} are shown both before (black line) and after (red line) being outcrossed to w¹¹¹⁸ (Sh⁺). In Sh¹⁰², Sh¹³³, and Sh^{M} flies daily sleep amount before the outcrossing was (min/day, mean±SEM) 593±17, 890±27, and 705±22, respectively. After the outcrossing it was 181±36, 427±29, and 274±28, respectively. Wild-type CS and white¹¹¹⁸ flies (thick black lines) are shown for comparison. The blue line indicates ss flies. For each line at least 30 flies were tested in 2 independent experiments.

FIGS. 13A-B—The injection of anti-Kv1.2 on the right cerebral cortex causes a significant and prolonged decrease of slow waves on the side of the injection. Slow waves are the most prominent marker of slow wave sleep, and their presence can be quantified by using power spectrum analysis of the EEG signal. Slow waves correspond to the frequency band of 0.5-4 Hz. In FIG. 13B, note that other frequency bands outside the slow waves range are not affected by the anti-Kv1.2 unilateral injection.

### DESCRIPTION OF ILLUSTRATIVE EMBODIMENTS

As discussed above, the response to sleep and sleep deprivation is likely a complex one that affects many different aspects of an individual's overall waking performance. Unfortunately, for many endeavors—military, airline, medical and emergency, and security industries to name a few—it is common for personnel to work extremely long hours and to have extended periods where they are given only brief opportunities for sleep. Thus, the present invention seeks to provide compositions and methods for addressing the need for sleep response-modifying therapies.

The present invention is based in part on the inventors' discovery of various genes that are associated with a diminished sleep requirement and normal continuous performance in Drosophila. Using the fruit fly as an experimental model, the inventors have identified genes that are associated with a diminished sleep requirement as well as those that permit to maintain a normal level of performance after sleep deprivation.

The fruit fly, Drosophila melanogaster, has been used as an ubiquitous model for the characterization of cellular processes (e.g., signaling pathways) involved in a variety of human diseases. In fact, the cellular functions of many genes known to be affected in human diseases were initially identified in Drosophila (see e.g., Holley et al., 1997). This high degree of conservation of morphogenetic processes between Drosophila and humans has made Drosophila a prime model system for the identification of putative drug targets using function based genetic approaches. The fruit fly is now an established model system to study gene-related disorders in humans. Extensive genome database resources (13,600 genes sequenced and annotated) are available. In addition, fruit fly genetics is simple enough to perform rapid mutagenesis, screenings, and breedings to elucidate the genetics of particular disorders.

The majority of fly genes are shared with humans. In fact, it is becoming increasingly apparent that the vertebrate genome arose from the amplification of a core set of genes not much larger than that of the fly. The majority (77%) of the genes involved in human diseases have fly counterparts (Reiter et al., 2001), and the expression of human genes into flies very often results in phenotypes that mimic human diseases (e.g., human α-synuclein in Drosophila causes a phenotype that resembles human Parkinson's disease; Auluck et al., 2002). In addition to myriad similarities in cellular structure and function, humans and flies share pathways for intercellular and intracellular signaling (from membrane receptors and ion channels to nuclear transcription factors), developmental patterning, learning and behavior, as well as tumor formation and metastasis, to give just a few examples (Littleton and Ganetzky, 2000). Thus, flies are now taken as simplified versions of vertebrate animals rather than simply as models of themselves.

Flies are neuro-biologically complex organisms with some 250,000 neurons. The fruit fly sleeps and needs sleep in much the same way as humans and other mammals do (Shaw et al., 2000). Fruit fly sleep, like sleep in mammals, is characterized by increased arousal threshold, changes in brain electrical activity, and is homeostatically regulated independent of the circadian clock. Also as in mammals, sleep is abundant in young flies and it is reduced in older flies, and is modulated by stimulants such as caffeine and hypnotics. Finally, several molecular markers modulated by sleep and wakefulness in mammals are also modulated by behavioral state in Drosophila.

The present inventors' have discovered various Drosophila genes that are associated with a diminished sleep requirement and normal continuous performance. In particular, the inventors have devised extensive experimental measurements of brain activity (EEG-like recordings), monitoring of locomotor activity, and vigilance tests that were implemented simultaneously in thousands of fruit flies. Based on these studies, they have now identified candidate genes that are associated with continuous performance in the fruit fly. Eighteen “short sleeper” and 5 sleep deprivation-resistant lines (in which sleep deprivation that does not result in low performance) were identified. Most of the affected genes associated with these lines have been identified. The sequence of these genes, and in some cases the function of the genes, are known. For others, this is the first report of functional significance.

The details of the present invention are described in the following pages.

A. Drosophila melanogaster

1. Basics

Drosophila melanogaster is a fruit fly, an insect about 3 mm long, of the kind that accumulates around spoiled fruit. It is also one of the most valuable of organisms in biological research, particularly in genetics and developmental biology. Drosophila has been used as a model organism for research for almost a century, and today, several thousand scientists are working on many different aspects of the fruit fly. Its importance for human health was recognized by the award of the Nobel Prize in medicine/physiology to Ed Lewis, Christiane Nusslein-Volhard and Eric Wieschaus in 1995.

Part of the reason people work on Drosophila is historical—so much is already known about it that it is easy to handle and well-understood. Part of it is practical—it is a small organism with a short life cycle of just two weeks, and is cheap and easy to keep large numbers. Mutant flies, with defects in any of several thousand genes are available, and the entire genome has recently been sequenced. Together, these advantages draw many researchers to use this system for a wide variety of scientific endeavors.

The Drosophila egg is about half a millimeter long. It takes about one day after fertilization for the embryo to develop and hatch into a worm-like larva. The larva eats and grows continuously, molting one day, two days, and four days after hatching (first, second and third instars). After two days as a third instar larva, it molts one more time to form an immobile pupa. Over the next four days, the body is completely remodeled to give the adult winged form, which then hatches from the pupal case and is fertile after another day (timing is for 25° C.; at 18° C., development takes twice as long).

Drosophila is so popular that, it would be almost impossible to list the number of things that are being done with it. Originally, it was mostly used in genetics, for example, to discover that genes were related to proteins and to study the rules of genetic inheritance. More recently, it has been used extensively in developmental biology, looking to see how a complex organism arises from a relatively simple fertilized egg. Embryonic development is where most of the attention is concentrated, but there is also a great deal of interest in how various adult structures develop in the pupa, mostly focused on the development of the compound eye, but also on the wings, legs and other organs.

Drosophila has four pairs of chromosomes: the X/Y sex chromosomes and the autosomes 2, 3, and 4. The fourth chromosome is quite tiny and rarely heard from. The size of the genome is about 165 million bases and contains and estimated 14,000 genes (by comparison, the human genome has 3,300 million bases and may have about 40,000 genes; yeast has about 5800 genes in 13.5 million base bases). The genome is now completely sequenced, and analysis of the data continues.

Polytene chromosomes are the magic markers that first put Drosophila in the spotlight. As the fly larva grows, it keeps the same number of cells, but needs to make much more gene product. The result is that the cells get much bigger and each chromosome divides hundreds of times, but all the strands stay attached to each other. The result is a massively thick polytene chromosome, which can easily be seen under the microscope. Even better, these chromosomes have a pattern of dark and light bands, like a bar code, which is unique for each section of the chromosome. As a result, by reading polytene bands, one can see what part of the chromosome one is looking at. Any large deletions, or other rearrangements of part of a chromosome can be identified, and using modern nucleic acid probes, individual cloned genes can be placed on the polytene map.

The standard map of the polytene chromosome divides the genome into 102 numbered bands (1-20 is the X, 21-60 is the second, 61-100 the third and 101-102 the fourth); each of those is divided into six letter bands (A-F) and those are subdivided into up to 13 numbered divisions. The location of many genes is known to the resolution of a letter band, usually with a guess to the number location (e.g., 42C7-9, 60A1-2). The polytene divisions do not have exactly the same length of sequence in them, but on average, a letter band contains about 300 kB of DNA, and 15-25 genes.

2. Nomenclature

The rules of Drosophila nomenclature are well defined and reasonably straightforward. For example, each gene has both a unique name and a unique gene symbol that is usually shorter than the name and contains no spaces, allowing genotypes to be described in an unambiguous and manageable way. Both are italicized in print. In general, genes are named in one of three ways. First, according to a mutant phenotype of the gene (generally the phenotype of the first mutant allele identified), e.g., white (w), Shaker (Sh), and cubitus interruptus (ci). The name and symbol are capitalized if the phenotype of the mutant allele for which the gene was named is dominant to a wild-type allele. Be aware, however, that many nominally ‘dominant genes’ have recessive alleles and many ‘recessive genes’ have dominant alleles.

Second, genes may be named according to a category of phenotypic effect, such as suppressor, enhancer, Minute, lethal, sterile, along with identifying information relevant to the class (the name of the gene that is suppressed or enhanced, or the chromosomal location of Minutes, lethals and steriles). Examples include: suppressor of forked (su(f)), Enhancer of Star (E(S)), Minute (1)15D (M(1)15D), lethal (3)85Ea (1(3)85Ea), and male sterile (2)1 (ms(2)1).

Third, when the product of a gene is known, the gene is typically named according to the product encoded, with a chromosomal location or series number if part of a multigene family. Examples include Tubulin (3)67C (Tub67C), Superoxide dismutase (Sod), and transfer RNA arginine (tRNA-Arg1). Superscripts identify individual mutant alleles of a gene: w^{a}, l(2)40Fg¹, Antp^{LC}. A “+” superscript indicates a wild-type allele of the gene. A “+” in place of a gene symbol indicates that the chromosome or the complete genotype, depending on the context, is wild type.

Chromosome aberrations are named according to the type of rearrangement, the chromosome or chromosomes involved, and an identifying symbol. The basic types of aberrations and their abbreviations are: deficiency (Df), duplication (Dp), inversion (In), transposition (Tp), translocation (T), compound (C), ring (R), levosynaptic element (LS) and dextrosynaptic element (DS). These are written as: Type(Chromosome)Identifier. The identifier may or may not convey information about the rearrangement. For example, Df(3R)by10 is the name of a deficiency in the right arm of the third chromosome; in this case the identifier reflects the inclusion of the blistery (by) gene within the deficiency and the 10 distinguishes it from others in a series. Superscripts, which define unique alleles, are not used with symbols of genes deleted by deficiencies (they are used only when the gene is interrupted, rather than removed, by the aberration). Df(3R)by10, Df(3R)by62, and Df(3R)by77 represent three unique deficiencies, but not unique alleles of by—the gene is equally absent in all three aberrations. T(2;3)ap^{Xa} refers to a translocation between chromosomes 2 and 3; here the translocation is named for the mutant allele of the apterous gene that results from one of the translocation breakpoints. Tp(1;3)O4 names a three-break event that resulted in the insertion of a piece of chromosome 1 into chromosome 3. In this case the identifier, O4, is arbitrary, formed from the name of the person who recovered the aberration and a series number.

Balancers are an important class of aberration and one for which shorthand is commonly used. Lindsley & Zimm (1992) define a set of core balancer symbols that are commonly used to represent a particular set of aberrations and markers. The most popular balancers exist in a variety of marker combinations, all with at least one dominant visible marker. There are three different standard ways of representing balancer chromosomes. (1) balancer symbol—a single symbol represents a unique set of aberrations and markers, e.g., TM3-Sb; (2) balancer short genotype—a core balancer symbol is combined with aberration, transposon and allele symbols to describe a unique balancer variant, e.g., TM3, Sb[1]; and (3) balancer full genotype—all aberration, transposon and allele symbols that comprise the unique balancer variant are explicitly stated, e.g., In(3LR)TM3, kni^{ri-1} p^{p} sep¹ l(3)89Aa¹ Sb¹ Ubx^{bx-}^{34e} e¹.

Transposon nomenclature has four basic parts: source of transposon ends, included genes, construct symbol, and insertion identifier. A transposon symbol is composed of ends{symbol}. A full transposon genotype adds the gene^{allele} symbols of all included genes, with the form ends{genes=symbol}. The symbol for a specific insertion of a given transposon has the form ends{symbol}identifier.

A properly assembled genotype represents all mutant components of the stock in the order 1;Y;2;3;4. Within a chromosome, aberrations precede gene symbols. A comma and space separate aberrations from gene symbols and genes are listed in the left-right order of the unrearranged chromosome. Gene symbols are separated by a space. Homologues are separated by a solidus (/) and heterologues are separated by a semicolon. Homozygous chromosomes are defined only once: cn bw implies cn bw/cn bw, and + implies +/+.

For example: (a) cv¹; sp¹; th¹—the stock is homozygous for three recessive mutations, crossveinless 1 on chromosome 1, speck 1 on 2, and thread 1 on 3; (b) In(1)dl-49+B^{M1}, sc¹ v^{Of}—the stock is homozygous for two inversions on the X, delta-49 and Bar of Muller, and two recessive mutations, scute 1 and vermillion Of; (c) Df(3L)emc5, red¹ /TM2, emc² p^{p} Ubx¹ e^{s}—the stock is heterozygous for a deficiency on the left arm of chromosome 3 that includes the extra macrochaetae gene, and also carries a mutation in the red gene (adults will express the recessive emc phenotype as well as the dominant Ubx phenotype because the balancer carries a mutant allele of emc in addition to the standard TM2 markers pink peach, Ultrabithorax 1, and ebony sooty); (d) T(2;3)CyOTM6, CyO: TM6/pr¹ cn¹ Adh^{ufs}; mwh¹ ry⁵⁰⁶ e¹—a translocation is superimposed on two balancer chromosomes, CyO and TM6 (the normal sequence homologues carry mutations in the 2nd chromosome genes purple, cinnabar and Alcohol dehydrogenase and the 3rd chromosome genes multiple wing hair, rosy and ebony); and (e) y¹ w¹¹¹⁸ P{ry^{+7.2}=hsFLP}1; TM3, ry^{RK} Sb¹ Ser¹/TM6B, ry^{CB} Tb¹ ca¹—this stock carries mutant alleles of yellow and white on the X as well as a P element transposon that is marked with a functionally wild-type allele of the rosy gene, as well an allele that expresses the yeast FLP gene, but in most cases only visible markers are shown in transposon genotypes (the symbol for this specific construct is hsFLP, and the identifier for this particular insertion of the hsFLP construct is 1).

The rules for designating autosomal homologues can't be strictly applied to sex chromosomes. Sometimes the genotypes of both sexes are explicitly defined, using the form X/X x X/Y or X/X & X/Y. More often a condensed notation is used and it is left to the user to apply the rules of segregation and sex determination to identify the genotype of each sex. For example, compound 1st, or attached-X, chromosomes are commonly used to create balanced stocks of X-linked female sterile mutations. In a stock of the female sterile mutation diminutive 1, the genotypes of males and females are dm¹/Y and C(1)DX/Y, respectively, but the stock genotype is usually written as dm¹/C(1)DX. The latter seems to imply a stock of triplo-X flies, but triplo-X metafemales have extremely low viability and survivors are sterile. The only interpretation consistent with the biology is that females carry a maternally inherited compound X, males carry a paternally inherited dm¹ X, and both sexes carry a wild-type Y chromosome inherited from the opposite sex.

B. Peptides and Polypeptides

In one aspect of the invention, previously unknown polypeptides that are involved with sleep are provided in an isolated and purified state. A list of these genes is provided in TABLE 1, and are included in the sequence listing as SEQ ID NOS:2, 4, 6, 8, 10, 12, 14, 16, 18, 20, 22, 24, 26, 28, 30, 54 and 55.

TABLE 1
Novel Sleep Gene List

| | | Cytogenetic | |
|---|---|---|---|
| Fly lines | Gene name | map | SEQ ID NOS. |
| EP(2)2508 (24/7) | CG18190, Jheh 1 | 55F7 | 1(2), 3(4) |
| 2p32 | CG7228 | 28D3 | 5(6) |
| 3p161 | lama | 64C15 | 7(8) |
| 5682 | disco | 14B1 | 9(10) |
| 13303 | CG6664 | 73E4 | 11(12) |
| 12739 | Casein kinase II | 10E3 | 13(14) |
| | β subunit | | |
| 12748 | CG9171 | 25F4 | 15(16) |
| 12832 | GstE1 | 55C6 | 17(18) |
| EP(2)2162) | cAMP-dependent | 46D1 | 19(20) |
| | protein kinase R2 | | |
| EP(2)2221 | CG15161, MESR3 | 36F7-9 | 21(22), 23(24) |
| EP(3)3717 | Meics | 70C7 | 25(26) |
| Df 3357 | Atpalpha, Calx, Rlip | 93B2-5 | 27(28-30), |
| | | | 31(32), 33(34) |
| Df 3788 | nompC, H15, Lam, | 25D6-F5 | 35(36), 37(38) |
| | Glu-RIIA, Glu-RIIB | | 39(40), 41(42) |
| | | | 43(44) |
| Df 5707 | Ork1 | 9F7-8 | 45(46) |
| EMS 1174, 1179 | Shaker | 16F4-5 | 47(48-52) |
| Hk1, Hkx | Hyperkinetic | 9B5 | vvv |

Other embodiments of the present invention pertain to isolated and purified peptides of about 10 to no more than about 50 amino acids in length comprising a segment of 10 or more residues from the polypeptide discussed above. These peptides (or fragments) of the polypeptides that may or may not retain various of the functions discussed above. Peptides may be produced de novo using chemical synthesis. Fragments, including the N-terminus of the molecule may be generated by genetic engineering of translation stop sites within the coding region (discussed below). Alternatively, treatment of the polypeptide with proteolytic enzymes, known as proteases, can produce a variety of N-terminal, C-terminal and internal fragments. Examples of fragments may include contiguous residues of SEQ ID NOS:2, 4, 6, 8, 10, 12, 14, 16, 18, 20, 22, 24, 26, 28-30, 32, 34, 36, 38, 40, 42, 44, 46, 48, 54 or 55 that are 10, 11, 12, 13, 14, 15, 16, 17, 18, 19, 20, 21, 22, 23, 24, 25, 30, 35, 40, 45, 50, 55, 60, 65, 75, 80, 85, 90, 95, 100, 200, 300, 400 or more amino acids in length. These fragments may be purified according to known methods, such as precipitation (e.g., ammonium sulfate), HPLC, ion exchange chromatography, affinity chromatography (including immunoaffinity chromatography) or various size separations (sedimentation, gel electrophoresis, gel filtration).

1. Variants of Polypeptides

Amino acid sequence variants of the polypeptides of the present invention can be substitutional, insertional or deletion variants. Deletion variants lack one or more residues of the native protein which are not essential for function or immunogenic activity, and are exemplified by the variants lacking a transmembrane sequence described above. Another common type of deletion variant is one lacking secretory signal sequences or signal sequences directing a protein to bind to a particular part of a cell. Insertional mutants typically involve the addition of material at a non-terminal point in the polypeptide. This may include the insertion of an immunoreactive epitope or simply a single residue. Terminal additions, called fusion proteins, are discussed below.

Substitutional variants typically contain the exchange of one amino acid for another at one or more sites within the protein, and may be designed to modulate one or more properties of the polypeptide, such as stability against proteolytic cleavage, without the loss of other functions or properties. Substitutions of this kind preferably are conservative, that is, one amino acid is replaced with one of similar shape and charge. Conservative substitutions are well known in the art and include, for example, the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine; methionine to leucine or isoleucine; phenylalanine to tyrosine, leucine or methionine; serine to threonine; threonine to serine; tryptophan to tyrosine; tyrosine to tryptophan or phenylalanine; and valine to isoleucine or leucine.

The following is a discussion based upon changing of the amino acids of a protein to create an equivalent, or even an improved, second-generation molecule. For example, certain amino acids may be substituted for other amino acids in a protein structure without appreciable loss of interactive binding capacity with structures such as, for example, antigen-binding regions of antibodies or binding sites on substrate molecules. Since it is the interactive capacity and nature of a protein that defines that protein's biological functional activity, certain amino acid substitutions can be made in a protein sequence, and its underlying DNA coding sequence, and nevertheless obtain a protein with like properties. It is thus contemplated by the inventors that various changes may be made in the DNA sequences of genes without appreciable loss of their biological utility or activity, as discussed below. Table 1 shows the codons that encode particular amino acids.

In making such changes, the hydropathic index of amino acids may be considered. The importance of the hydropathic amino acid index in conferring interactive biologic function on a protein is generally understood in the art (Kyte and Doolittle, 1982). It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like.

Each amino acid has been assigned a hydropathic index on the basis of their hydrophobicity and charge characteristics (Kyte and Doolittle, 1982), these are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

It is known in the art that certain amino acids may be substituted by other amino acids having a similar hydropathic index or score and still result in a protein with similar biological activity, i.e., still obtain a biological functionally equivalent protein. In making such changes, the substitution of amino acids whose hydropathic indices are within ±2 is preferred, those which are within ±1 are particularly preferred, and those within ±0.5 are even more particularly preferred.

It is also understood in the art that the substitution of like amino acids can be made effectively on the basis of hydrophilicity. U.S. Pat. No. 4,554,101, incorporated herein by reference, states that the greatest local average hydrophilicity of a protein, as governed by the hydrophilicity of its adjacent amino acids, correlates with a biological property of the protein. As detailed in U.S. Pat. No. 4,554,101, the following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine *−0.5); cysteine (−1.0); methionine (−1.3); valine (−1.5); leucine (−1.8); isoleucine (−1.8); tyrosine (−2.3); phenylalanine (−2.5); tryptophan (−3.4).

It is understood that an amino acid can be substituted for another having a similar hydrophilicity value and still obtain a biologically equivalent and immunologically equivalent protein. In such changes, the substitution of amino acids whose hydrophilicity values are within ±2 is preferred, those that are within ±1 are particularly preferred, and those within ±0.5 are even more particularly preferred.

As outlined above, amino acid substitutions are generally based on the relative similarity of the amino acid side-chain substituents, for example, their hydrophobicity, hydrophilicity, charge, size, and the like. Exemplary substitutions that take various of the foregoing characteristics into consideration are well known to those of skill in the art and include: arginine and lysine; glutamate and aspartate; serine and threonine; glutamine and asparagine; and valine, leucine and isoleucine.

Another embodiment for the preparation of polypeptides according to the invention is the use of peptide mimetics. Mimetics are peptide-containing molecules that mimic elements of protein secondary structure (Johnson et al., 1993). The underlying rationale behind the use of peptide mimetics is that the peptide backbone of proteins exists chiefly to orient amino acid side chains in such a way as to facilitate molecular interactions, such as those of antibody and antigen. A peptide mimetic is expected to permit molecular interactions similar to the natural molecule. These principles may be used, in conjunction with the principles outline above, to engineer second generation molecules having many of the natural properties of the polypeptides of SEQ ID NOS:2, 4, 6, 8, 10, 12, 14, 16, 18, 20, 22, 24, 26, 28-30, 32, 34, 36, 38, 40, 42, 44, 46, 48, 54, or 55 but with altered and even improved characteristics.

2. Domain Switching

Domain switching involves the generation of chimeric molecules using different but, in this case, related polypeptides, for example, homologs from different species. These molecules may have additional value in that these “chimeras” can be distinguished from natural molecules, while possibly providing the same function.

3. Fusion Proteins

A specialized kind of insertional variant is the fusion protein. This molecule generally has all or a substantial portion of the native molecule, linked at the N- or C-terminus, to all or a portion of a second polypeptide. For example, fusions typically employ leader sequences from other species to permit the recombinant expression of a protein in a heterologous host. Another useful fusion includes the addition of a immunologically active domain, such as an antibody epitope, to facilitate purification of the fusion protein. Inclusion of a cleavage site at or near the fusion junction will facilitate removal of the extraneous polypeptide after purification. Other useful fusions include linking of functional domains, such as active sites from enzymes, glycosylation domains, cellular targeting signals or transmembrane regions.

4. Purification of Proteins

It will be desirable to purify the novel polypeptide sequences of the present invention or variants thereof. Protein purification techniques are well known to those of skill in the art. These techniques involve, at one level, the crude fractionation of the cellular milieu to polypeptide and non-polypeptide fractions. Having separated the polypeptide from other proteins, the polypeptide of interest may be further purified using chromatographic and electrophoretic techniques to achieve partial or complete purification (or purification to homogeneity). Analytical methods particularly suited to the preparation of a pure peptide are ion-exchange chromatography, exclusion chromatography; polyacrylamide gel electrophoresis; isoelectric focusing. A particularly efficient method of purifying peptides is fast protein liquid chromatography or even HPLC.

Certain aspects of the present invention concern the purification, and in particular embodiments, the substantial purification, of an encoded protein or peptide. The term “purified protein or peptide” as used herein, is intended to refer to a composition, isolatable from other components, wherein the protein or peptide is purified to any degree relative to its naturally-obtainable state. A purified protein or peptide therefore also refers to a protein or peptide, free from the environment in which it may naturally occur.

Generally, “purified” will refer to a protein or peptide composition that has been subjected to fractionation to remove various other components, and which composition substantially retains its expressed biological activity. Where the term “substantially purified” is used, this designation will refer to a composition in which the protein or peptide forms the major component of the composition, such as constituting about 50%, about 60%, about 70%, about 80%, about 90%, about 95% or more of the proteins in the composition.

Various methods for quantifying the degree of purification of the protein or peptide will be known to those of skill in the art in light of the present disclosure. These include, for example, determining the specific activity of an active fraction, or assessing the amount of polypeptides within a fraction by SDS/PAGE analysis. A preferred method for assessing the purity of a fraction is to calculate the specific activity of the fraction, to compare it to the specific activity of the initial extract, and to thus calculate the degree of purity, herein assessed by a “-fold purification number.” The actual units used to represent the amount of activity will, of course, be dependent upon the particular assay technique chosen to follow the purification and whether or not the expressed protein or peptide exhibits a detectable activity.

Various techniques suitable for use in protein purification will be well known to those of skill in the art. These include, for example, precipitation with ammonium sulphate, PEG, antibodies and the like or by heat denaturation, followed by centrifugation; chromatography steps such as ion exchange, gel filtration, reverse phase, hydroxylapatite and affinity chromatography; isoelectric focusing; gel electrophoresis; and combinations of such and other techniques. As is generally known in the art, it is believed that the order of conducting the various purification steps may be changed, or that certain steps may be omitted, and still result in a suitable method for the preparation of a substantially purified protein or peptide.

There is no general requirement that the protein or peptide always be provided in their most purified state. Indeed, it is contemplated that less substantially purified products will have utility in certain embodiments. Partial purification may be accomplished by using fewer purification steps in combination, or by utilizing different forms of the same general purification scheme. For example, it is appreciated that a cation-exchange column chromatography performed utilizing an HPLC apparatus will generally result in a greater “-fold” purification than the same technique utilizing a low pressure chromatography system. Methods exhibiting a lower degree of relative purification may have advantages in total recovery of protein product, or in maintaining the activity of an expressed protein.

It is known that the migration of a polypeptide can vary, sometimes significantly, with different conditions of SDS/PAGE (Capaldi et al., 1977). It will therefore be appreciated that under differing electrophoresis conditions, the apparent molecular weights of purified or partially purified expression products may vary.

High Performance Liquid Chromatography (HPLC) is characterized by a very rapid separation with extraordinary resolution of peaks. This is achieved by the use of very fine particles and high pressure to maintain an adequate flow rate. Separation can be accomplished in a matter of minutes, or at most an hour. Moreover, only a very small volume of the sample is needed because the particles are so small and close-packed that the void volume is a very small fraction of the bed volume. Also, the concentration of the sample need not be very great because the bands are so narrow that there is very little dilution of the sample.

Gel chromatography, or molecular sieve chromatography, is a special type of partition chromatography that is based on molecular size. The theory behind gel chromatography is that the column, which is prepared with tiny particles of an inert substance that contain small pores, separates larger molecules from smaller molecules as they pass through or around the pores, depending on their size. As long as the material of which the particles are made does not adsorb the molecules, the sole factor determining rate of flow is the size. Hence, molecules are eluted from the column in decreasing size, so long as the shape is relatively constant. Gel chromatography is unsurpassed for separating molecules of different size because separation is independent of all other factors such as pH, ionic strength, temperature, etc. There also is virtually no adsorption, less zone spreading and the elution volume is related in a simple matter to molecular weight.

Affinity Chromatography is a chromatographic procedure that relies on the specific affinity between a substance to be isolated and a molecule that it can specifically bind to. This is a receptor-ligand type interaction. The column material is synthesized by covalently coupling one of the binding partners to an insoluble matrix. The column material is then able to specifically adsorb the substance from the solution. Elution occurs by changing the conditions to those in which binding will not occur (alter pH, ionic strength, temperature, etc.).

A particular type of affinity chromatography useful in the purification of carbohydrate containing compounds is lectin affinity chromatography. Lectins are a class of substances that bind to a variety of polysaccharides and glycoproteins. Lectins are usually coupled to agarose by cyanogen bromide. Conconavalin A coupled to Sepharose was the first material of this sort to be used and has been widely used in the isolation of polysaccharides and glycoproteins; other lectins that have been include lentil lectin and wheat germ agglutinin, which has been useful in the purification of N-acetyl glucosaminyl residues and Helix pomatia lectin. Lectins themselves are purified using affinity chromatography with carbohydrate ligands. Lactose has been used to purify lectins from castor bean and peanuts; maltose has been useful in extracting lectins from lentils and jack bean; N-acetyl-D galactosamine is used for purifying lectins from soybean; N-acetyl glucosaminyl binds to lectins from wheat germ; D-galactosamine has been used in obtaining lectins from clams and L-fucose will bind to lectins from lotus.

The matrix should be a substance that itself does not adsorb molecules to any significant extent and that has a broad range of chemical, physical and thermal stability. The ligand should be coupled in such a way as to not affect its binding properties. The ligand should also provide relatively tight binding. And it should be possible to elute the substance without destroying the sample or the ligand. One of the most common forms of affinity chromatography is immunoaffinity chromatography. The generation of antibodies that would be suitable for use in accord with the present invention is discussed below.

5. Synthetic Peptides

As discussed above, the present invention encompasses peptides of the larger polypeptide sequences. Because of their relatively small size, the peptides of the invention can also be synthesized in solution or on a solid support in accordance with conventional techniques. Various automatic synthesizers are commercially available and can be used in accordance with known protocols. See, e.g., Stewart and Young (1984); Tam et al. (1983); Merrifield (1986); and Barany and Merrifield (1979), each incorporated herein by reference. Short peptide sequences, or libraries of overlapping peptides, usually from about 6 up to about 35 to 50 amino acids, which correspond to the selected regions described herein, can be readily synthesized and then screened in screening assays designed to identify reactive peptides. Alternatively, recombinant DNA technology may be employed wherein a nucleotide sequence which encodes a peptide of the invention is inserted into an expression vector, transformed or transfected into an appropriate host cell and cultivated under conditions suitable for expression.

6. Antigen Compositions and Antibody Generation

The present invention also provides for the use of proteins, polypeptides, or peptides as antigens for the immunization of animals relating to the production of antibodies. It is proposed that the antibodies of the present invention will find useful application in standard immunochemical procedures, such as ELISA and Western blot methods and in immunohistochemical procedures such as tissue staining, as well as in other procedures which may utilize antibodies specific to antigen epitopes.

Thus, certain embodiments of the present invention pertain to a polyclonal antisera, antibodies of which bind immunologically to a polypeptide selected from the group consisting of SEQ ID NOS:2, 4, 6, 8, 10, 12, 14, 16, 18, 20, 22, 24, 26, 28-30, 32, 34, 36, 38, 40, 42, 44, 46, 48, 54, or 55. In other embodiments, the invention pertains to a monoclonal antibody that immunologically binds to a polypeptide selected from the group consisting of SEQ ID NOS:2, 4, 6, 8, 10, 12, 14, 16, 18, 20, 22, 24, 26, 28-30, 32, 34, 36, 38, 40, 42, 44, 46, 48, 54, or 55.

Polyclonal sera is prepared by immunizing an animal with an immunogen comprising a polypeptide of the present invention and collecting antisera from that immunized animal. A wide range of animal species can be used for the production of antisera. Typically an animal used for production of anti-antisera is a non-human animal including rabbits, mice, rats, hamsters, pigs or horses. Because of the relatively large blood volume of rabbits, a rabbit is a preferred choice for production of polyclonal antibodies.

As is well known in the art, a given composition may vary in its immunogenicity. It is often necessary therefore to boost the host immune system, as may be achieved by coupling a peptide or polypeptide immunogen to a carrier. Exemplary and preferred carriers are keyhole limpet hemocyanin (KLH) and bovine serum albumin (BSA). Other albumins such as ovalbumin, mouse serum albumin or rabbit serum albumin can also be used as carriers. Means for conjugating a polypeptide to a carrier protein are well known in the art and include glutaraldehyde, m-maleimidobencoyl-N-hydroxysuccinimide ester, carbodiimide and bis-biazotized benzidine.

It is envisioned that SEQ ID NOS:2, 4, 6, 8, 10, 12, 14, 16, 18, 20, 22, 24, 26, 28-30, 32, 34, 36, 38, 40, 42, 44, 46, 48, 54, or 55 or portions thereof, will be coupled, bonded, bound, conjugated or chemically-linked to one or more agents via linkers, polylinkers or derivatized amino acids. This may be performed such that a bispecific or multivalent composition or vaccine is produced. It is further envisioned that the methods used for administration to animals, i.e., pharmaceutically acceptable, will be familiar to those of skill in the art.

As also is well known in the art, the immunogenicity of a particular immunogen composition can be enhanced by the use of non-specific stimulators of the immune response, known as adjuvants. Exemplary and preferred adjuvants include complete Freund's adjuvant (a non-specific stimulator of the immune response containing killed Mycobacterium tuberculosis), incomplete Freund's adjuvants and aluminum hydroxide adjuvant.

The amount of immunogen composition used in the production of polyclonal antibodies varies upon the nature of the immunogen as well as the animal used for immunization. A variety of routes can be used to administer the immunogen (subcutaneous, intramuscular, intradermal, intravenous and intraperitoneal). The production of polyclonal antibodies may be monitored by sampling blood of the immunized animal at various points following immunization. A second, booster, injection may also be given. The process of boosting and titering is repeated until a suitable titer is achieved. When a desired level of immunogenicity is obtained, the immunized animal can be bled and the serum isolated and stored, and/or the animal can be used to generate mAbs.

MAbs may be readily prepared through use of well-known techniques, such as those exemplified in U.S. Pat. No. 4,196,265, incorporated herein by reference. Typically, this technique involves immunizing a suitable animal with a selected immunogen composition, e.g., a purified or partially purified protein, polypeptide or peptide. The immunizing composition is administered in a manner effective to stimulate antibody producing cells. Rodents such as mice and rats are preferred animals, however, the use of rabbit, sheep frog cells is also possible. The use of rats may provide certain advantages (Goding, 1986), but mice are preferred, with the BALB/c mouse being most preferred as this is most routinely used and generally gives a higher percentage of stable fusions.

Following immunization, somatic cells with the potential for producing antibodies, specifically B-lymphocytes (B-cells), are selected for use in the mAb generating protocol. These cells may be obtained from biopsied spleens, tonsils or lymph nodes, or from a peripheral blood sample. Spleen cells and peripheral blood cells are preferred, the former because they are a rich source of antibody-producing cells that are in the dividing plasmablast stage, and the latter because peripheral blood is easily accessible. Often, a panel of animals will have been immunized and the spleen of animal with the highest antibody titer will be removed and the spleen lymphocytes obtained by homogenizing the spleen with a syringe. Typically, a spleen from an immunized mouse contains approximately 5×10⁷ to 2×10⁸ lymphocytes.

The antibody-producing B lymphocytes from the immunized animal are then fused with cells of an immortal myeloma cell, generally one of the same species as the animal that was immunized. Myeloma cell lines suited for use in hybridoma-producing fusion procedures preferably are non-antibody-producing, have high fusion efficiency, and enzyme deficiencies that render then incapable of growing in certain selective media which support the growth of only the desired fused cells (hybridomas). Fusion procedures usually produce viable hybrids at low frequencies, around 1×10⁻⁶ to 1×10⁻⁸. However, this does not pose a problem, as the viable, fused hybrids are differentiated from the parental, unfused cells (particularly the unfused myeloma cells that would normally continue to divide indefinitely) by culturing in a selective medium.

Xelected hybridomas are serially diluted and cloned into individual antibody-producing cell lines, which clones can then be propagated indefinitely to provide mAbs. The cell lines may be exploited for mAb production in two basic ways. A sample of the hybridoma can be injected (often into the peritoneal cavity) into a histocompatible animal of the type that was used to provide the somatic and myeloma cells for the original fusion. The injected animal develops tumors secreting the specific monoclonal antibody produced by the fused cell hybrid. The body fluids of the animal, such as serum or ascites fluid, can then be tapped to provide mAbs in high concentration. The individual cell lines could also be cultured in vitro, where the mAbs are naturally secreted into the culture medium from which they can be readily obtained in high concentrations. mAbs produced by either means may be further purified, if desired, using filtration, centrifugation and various chromatographic methods such as HPLC or affinity chromatography.

In general, both polyclonal and monoclonal antibodies against the novel polypeptide sequences of the present invention may be used in a variety of embodiments. For example, they may be employed in antibody cloning protocols to obtain cDNAs or genes encoding other polypeptides associated with sleep regulation. They may also be used in inhibition studies to analyze the effects of related peptides in cells or animals. The antibodies of the present invention will also be useful in immunolocalization studies to analyze the distribution of SEQ ID NOS:2, 4, 6, 8, 10, 12, 14, 16, 18, 20, 22, 24, 26, 28-30, 32, 34, 36, 38, 40, 42, 44, 46, 48, 54, or 55 during various cellular events, for example, to determine the cellular or tissue-specific distribution of these polypeptides under different points in the cell cycle. A particularly useful application of such antibodies is in purifying native or recombinant polypeptides, for example, using an antibody affinity column. The operation of all such immunological techniques will be known to those of skill in the art in light of the present disclosure.

C. Nucleic Acids

The present invention also provides, in certain embodiments, isolated and purified nucleic acids that include a segment encoding a polypeptide selected from the group consisting of SEQ ID NOS: 1, 3, 5, 7, 9, 11, 13, 15, 17, 19, 21, 23, 25, 27, 31, 33, 35, 37, 39, 41, 43, 45, 47 and 53. In other embodiments, the invention provides for isolated and purified oligonucleotides of no more than about 50 nucleotides in length that include a segment of 15 or more consecutive bases from a polynucleotide selected from the group consisting of SEQ ID NOS: 1, 3, 5, 7, 9, 11, 13, 15, 17, 19, 21, 23, 25, 27, 31, 33, 35, 37, 39, 41, 43, 45, 47 and 53. The present invention is not limited in scope to these nucleic acids, however, as one of ordinary skill in the could, using these nucleic acids, readily identify related homologs in these and various other species (e.g., rat, rabbit, dog, monkey, gibbon, human, chimp, ape, baboon, cow, pig, horse, sheep, cat and other species).

The invention discloses specific polynucleotide sequences. It should be clear that the present invention is not limited to the specific nucleic acids disclosed herein. As discussed below, an equivalent polynucleotide may contain a variety of different bases and yet still produce a corresponding polypeptide that is functionally indistinguishable, and in some cases structurally, from the human and mouse genes disclosed herein.

Similarly, any reference to a nucleic acid should be read as encompassing a host cell containing that nucleic acid and, in some cases, capable of expressing the product of that nucleic acid. Cells comprising nucleic acids of the present invention may prove useful in the context of screening for agents that induce, repress, inhibit, augment, interfere with, block, abrogate, stimulate or enhance sleep or sleep response.

1. Nucleic Acids Encoding Novel Polypeptide Sequences

Nucleic acids according to the present invention may encode the entirety of SEQ ID NOS: 1, 3, 5, 7, 9, 11, 13, 15, 17, 19, 21, 23, 25, 27, 31, 33, 35, 37, 39, 41, 43, 45, 47 and 53 a domain of one of these sequences, or any other fragment of one of these sequences as set forth herein.

The nucleic acid may be derived from genomic DNA, i.e., cloned directly from the genome of a particular organism. In preferred embodiments, however, the nucleic acid would comprise complementary DNA (cDNA). Also contemplated is a cDNA plus a natural intron or an intron derived from another gene; such engineered molecules are sometime referred to as “mini-genes.” At a minimum, these and other nucleic acids of the present invention may be used as molecular weight standards in, for example, gel electrophoresis.

The term “cDNA” is intended to refer to DNA prepared using messenger RNA (mRNA) as template. The advantage of using a cDNA, as opposed to genomic DNA or DNA polymerized from a genomic, non- or partially-processed RNA template, is that the cDNA primarily contains coding sequences of the corresponding protein. There may be times when the full or partial genomic sequence is preferred, such as where the non-coding regions are required for optimal expression or where non-coding regions such as introns are to be targeted in an antisense strategy.

It also is contemplated that a given sequence from a given species may be represented by natural variants that have slightly different nucleic acid sequences but, nonetheless, encode the same protein (see Table 1 below).

As used in this application, the term “an isolated and purified nucleic acid” refers to a nucleic acid molecule that has been isolated free of total cellular nucleic acid. In preferred embodiments, the invention concerns a nucleic acid sequence essentially as set forth in SEQ ID NOS: 1, 3, 5, 7, 9, 11, 13, 15, 17, 19, 21, 23, 25, 27, 31, 33, 35, 37, 39, 41, 43, 45, 47 and 53. The term “as set forth in SEQ ID NOS: 1, 3, 5, 7, 9, 11, 13, 15, 17, 19, 21, 23, 25, 27, 31, 33, 35, 37, 39, 41, 43, 45, 47 and 53” means that the nucleic acid sequence substantially corresponds to a portion of SEQ ID NOS: 1, 3, 5, 7, 9, 11, 13, 15, 17, 19, 21, 23, 25, 27, 31, 33, 35, 37, 39, 41, 43, 45, 47 and 53. The term “functionally equivalent codon” is used herein to refer to codons that encode the same amino acid, such as the six codons for arginine or serine (TABLE 2, below), and also refers to codons that encode biologically equivalent amino acids, as discussed in the following pages.

TABLE 2

| | |
|---|---|
| Amino Acids | Codons |

| | | | |
|---|---|---|---|
| Alanine | Ala | A | GCA GCC GCG GCU |
| Cysteine | Cys | C | UGC UGU |
| Aspartic acid | Asp | D | GAC GAU |
| Glutamic acid | Glu | E | GAA GAG |
| Phenylalanine | Phe | F | UUC UUU |
| Glycine | Gly | G | GGA GGC GGG GGU |
| Histidine | His | H | CAC CAU |
| Isoleucine | Ile | I | AUA AUC AUU |
| Lysine | Lys | K | AAA AAG |
| Leucine | Leu | L | UUA UUG CUA CUC CUG CUU |
| Methionine | Met | M | AUG |
| Asparagine | Asn | N | AAC AAU |
| Proline | Pro | P | CCA CCC CCG CCU |
| Glutamine | Gln | Q | CAA CAG |
| Arginine | Arg | R | AGA AGG CGA CGC CGG CGU |
| Serine | Ser | S | AGC AGU UCA UCC UCG UCU |
| Threonine | Thr | T | ACA ACC ACG ACU |
| Valine | Val | V | GUA GUC GUG GUU |
| Tryptophan | Trp | W | UGG |
| Tyrosine | Tyr | Y | UAC UAU |

Allowing for the degeneracy of the genetic code, sequences that have at least about 50%, usually at least about 60%, more usually about 70%, most usually about 80%, preferably at least about 90% and most preferably about 95% of nucleotides that are identical to the nucleotides of SEQ ID NOS: 1, 3, 5, 7, 9, 11, 13, 15, 17, 19, 21, 23, 25, 27, 31, 33, 35, 37, 39, 41, 43, 45, 47 and 53 are contemplated. Sequences that are essentially the same as those set forth in SEQ ID NOS: 1, 3, 5, 7, 9, 11, 13, 15, 17, 19, 21, 23, 25, 27, 31, 33, 35, 37, 39, 41, 43, 45, 47 and 53 may also be functionally defined as sequences that are capable of hybridizing to a nucleic acid segment containing the complement of SEQ ID NOS: 1, 3, 5, 7, 9, 11, 13, 15, 17, 19, 21, 23, 25, 27, 31, 33, 35, 37, 39, 41, 43, 45, 47 and 53 under standard conditions.

The DNA segments of the present invention include those encoding biologically functional equivalent proteins and peptides, as described above. Such sequences may arise as a consequence of codon redundancy and amino acid functional equivalency that are known to occur naturally within nucleic acid sequences and the proteins thus encoded. Alternatively, functionally equivalent proteins or peptides may be created via the application of recombinant DNA technology, in which changes in the protein structure may be engineered, based on considerations of the properties of the amino acids being exchanged. Changes designed by man may be introduced through the application of site-directed mutagenesis techniques or may be introduced randomly and screened later for the desired function, as described below.

2. Oligonucleotide Probes and Primers

Naturally, the present invention also encompasses DNA segments that are complementary, or essentially complementary, to the sequence set forth in SEQ ID NOS: 1, 3, 5, 7, 9, 11, 13, 15, 17, 19, 21, 23, 25, 27, 31, 33, 35, 37, 39, 41, 43, 45, 47 and 53. Nucleic acid sequences that are “complementary” are those that are capable of base-pairing according to the standard Watson-Crick complementary rules. As used herein, the term “complementary sequences” means nucleic acid sequences that are substantially complementary, as may be assessed by the same nucleotide comparison set forth above, or as defined as being capable of hybridizing to the nucleic acid segment of SEQ ID NOS: 1, 3, 5, 7, 9, 11, 13, 15, 17, 19, 21, 23, 25, 27, 31, 33, 35, 37, 39, 41, 43, 45, 47 and 53 under relatively stringent conditions such as those described herein. Such sequences may encode entire proteins corresponding to SEQ ID NOS: 1, 3, 5, 7, 9, 11, 13, 15, 17, 19, 21, 23, 25, 27, 31, 33, 35, 37, 39, 41, 43, 45, 47 and 53 or functional or non-functional fragments thereof.

Alternatively, the hybridizing segments may be shorter oligonucleotides. Sequences of 17 bases long should occur only once in the human genome and, therefore, suffice to specify a unique target sequence. Although shorter oligomers are easier to make and increase in vivo accessibility, numerous other factors are involved in determining the specificity of hybridization. Both binding affinity and sequence specificity of an oligonucleotide to its complementary target increases with increasing length. It is contemplated that exemplary oligonucleotides of 8, 9, 10, 11, 12, 13, 14, 15, 16, 17, 18, 19, 20, 25, 30, 35, 40, 45, 50, 55, 60, 65, 70, 75, 80, 85, 90, 95, 100 or more base pairs will be used, although others are contemplated. Longer polynucleotides encoding 250, 500, 1000, 1212, 1500, 2000, 2500, 3000 or 5000 bases and longer are contemplated as well. Such oligonucleotides will find use, for example, as probes in Southern and Northern blots and as primers in amplification reactions.

Suitable hybridization conditions will be well known to those of skill in the art. In certain applications, for example, substitution of amino acids by site-directed mutagenesis, it is appreciated that lower stringency conditions are required. Under these conditions, hybridization may occur even though the sequences of probe and target strand are not perfectly complementary, but are mismatched at one or more positions. Conditions may be rendered less stringent by increasing salt concentration and decreasing temperature. For example, a medium stringency condition could be provided by about 0.1 to 0.25 M NaCl at temperatures of about 37° C. to about 55° C., while a low stringency condition could be provided by about 0.15 M to about 0.9 M salt, at temperatures ranging from about 20° C. to about 55° C. Thus, hybridization conditions can be readily manipulated, and thus will generally be a method of choice depending on the desired results.

In other embodiments, hybridization may be achieved under conditions of, for example, 50 mM Tris-HCl (pH 8.3), 75 mM KCl, 3 mM MgCl₂, 10 mM dithiothreitol, at temperatures between approximately 20° C. to about 37° C. Other hybridization conditions utilized could include approximately 10 mM Tris-HCl (pH 8.3), 50 mM KCl, 1.5 μM MgCl₂, at temperatures ranging from approximately 40° C. to about 72° C. Formamide and SDS also may be used to alter the hybridization conditions.

One method of using probes and primers of the present invention is in the search for nucleic acids related to SEQ ID NOS: 1, 3, 5, 7, 9, 11, 13, 15, 17, 19, 21, 23, 25, 27, 31, 33, 35, 37, 39, 41, 43, 45, 47 and 53 or, more particularly, homologs of these sequences from other species. Normally, the target DNA will be a genomic or cDNA library, although screening may involve analysis of RNA molecules. By varying the stringency of hybridization, and the region of the probe, different degrees of homology may be discovered.

Another way of exploiting probes and primers of the present invention is in s site-directed, or site-specific mutagenesis. Site-specific mutagenesis is a technique useful in the preparation of individual peptides, or biologically functional equivalent proteins or peptides, through specific mutagenesis of the underlying DNA. The technique further provides a ready ability to prepare and test sequence variants, incorporating one or more of the foregoing considerations, by introducing one or more nucleotide sequence changes into the DNA. Site-specific mutagenesis allows the production of mutants through the use of specific oligonucleotide sequences which encode the DNA sequence of the desired mutation, as well as a sufficient number of adjacent nucleotides, to provide a primer sequence of sufficient size and sequence complexity to form a stable duplex on both sides of the deletion junction being traversed. Typically, a primer of about 17 to 25 nucleotides in length is preferred, with about 5 to 10 residues on both sides of the junction of the sequence being altered.

The technique typically employs a bacteriophage vector that exists in both a single-stranded and double-stranded form. Typical vectors useful in site-directed mutagenesis include vectors such as the M13 phage. These phage vectors are commercially available and their use is generally well known to those skilled in the art. Double-stranded plasmids are also routinely employed in site directed mutagenesis, which eliminates the step of transferring the gene of interest from a phage to a plasmid.

In general, site-directed mutagenesis is performed by first obtaining a single-stranded vector, or melting of two strands of a double-stranded vector which includes within its sequence a DNA sequence encoding the desired protein. An oligonucleotide primer bearing the desired mutated sequence is synthetically prepared. This primer is then annealed with the single-stranded DNA preparation, taking into account the degree of mismatch when selecting hybridization conditions, and subjected to DNA polymerizing enzymes such as E. coli polymerase I Klenow fragment, in order to complete the synthesis of the mutation-bearing strand. Thus, a heteroduplex is formed wherein one strand encodes the original non-mutated sequence and the second strand bears the desired mutation. This heteroduplex vector is then used to transform appropriate cells, such as E. coli cells, and clones are selected that include recombinant vectors bearing the mutated sequence arrangement.

The preparation of sequence variants of the selected gene using site-directed mutagenesis is provided as a means of producing potentially useful species and is not meant to be limiting, as there are other ways in which sequence variants of genes may be obtained. For example, recombinant vectors encoding the desired gene may be treated with mutagenic agents, such as hydroxylamine, to obtain sequence variants.

3. Antisense Constructs

Antisense methodology takes advantage of the fact that nucleic acids tend to pair with “complementary” sequences. By complementary, it is meant that polynucleotides are those which are capable of base-pairing according to the standard Watson-Crick complementarity rules. That is, the larger purines will base pair with the smaller pyrimidines to form combinations of guanine paired with cytosine (G:C) and adenine paired with either thymine (A:T) in the case of DNA, or adenine paired with uracil (A:U) in the case of RNA. Inclusion of less common bases such as inosine, 5-methylcytosine, 6-methyladenine, hypoxanthine and others in hybridizing sequences does not interfere with pairing.

Targeting double-stranded (ds) DNA with polynucleotides leads to triple-helix formation; targeting RNA will lead to double-helix formation. Antisense polynucleotides, when introduced into a target cell, specifically bind to their target polynucleotide and interfere with transcription, RNA processing, transport, translation and/or stability. Antisense RNA constructs, or DNA encoding such antisense RNA's, may be employed to inhibit gene transcription or translation or both within a host cell, either in vitro or in vivo, such as within a host animal, including a human subject.

Antisense constructs may be designed to bind to the promoter and other control regions, exons, introns or even exon-intron boundaries of a gene. It is contemplated that the most effective antisense constructs will include regions complementary to intron/exon splice junctions. Thus, it is proposed that a preferred embodiment includes an antisense construct with complementarity to regions within 50-200 bases of an intron-exon splice junction. It has been observed that some exon sequences can be included in the construct without seriously affecting the target selectivity thereof. The amount of exonic material included will vary depending on the particular exon and intron sequences used. One can readily test whether too much exon DNA is included simply by testing the constructs in vitro to determine whether normal cellular function is affected or whether the expression of related genes having complementary sequences is affected.

As stated above, “complementary” or “antisense” means polynucleotide sequences that are substantially complementary over their entire length and have very few base mismatches. For example, sequences of fifteen bases in length may be termed complementary when they have complementary nucleotides at thirteen or fourteen positions. Naturally, sequences which are completely complementary will be sequences which are entirely complementary throughout their entire length and have no base mismatches. Other sequences with lower degrees of homology also are contemplated. For example, an antisense construct which has limited regions of high homology, but also contains a non-homologous region (e.g., ribozyme; see below) could be designed. These molecules, though having less than 50% homology, would bind to target sequences under appropriate conditions.

It may be advantageous to combine portions of genomic DNA with cDNA or synthetic sequences to generate specific constructs. For example, where an intron is desired in the ultimate construct, a genomic clone will need to be used. The cDNA or a synthesized polynucleotide may provide more convenient restriction sites for the remaining portion of the construct and, therefore, would be used for the rest of the sequence.

4. Ribozymes

Although proteins traditionally have been used for catalysis of nucleic acids, another class of macromolecules has emerged as useful in this endeavor. Ribozymes are RNA-protein complexes that cleave nucleic acids in a site-specific fashion. Ribozymes have specific catalytic domains that possess endonuclease activity (Kim and Cook, 1987; Gerlach et al., 1987; Forster and Symons, 1987). For example, a large number of ribozymes accelerate phosphoester transfer reactions with a high degree of specificity, often cleaving only one of several phosphoesters in an oligonucleotide substrate (Michel and Westhof, 1990; Reinhold-Hurek and Shub, 1992). This specificity has been attributed to the requirement that the substrate bind via specific base-pairing interactions to the internal guide sequence (“IGS”) of the ribozyme prior to chemical reaction.

Ribozyme catalysis has primarily been observed as part of sequence-specific cleavage/ligation reactions involving nucleic acids (Joyce, 1989). For example, U.S. Pat. No. 5,354,855 reports that certain ribozymes can act as endonucleases with a sequence specificity greater than that of known ribonucleases and approaching that of the DNA restriction enzymes. Thus, sequence-specific ribozyme-mediated inhibition of gene expression may be particularly suited to therapeutic applications (Scanlon et al., 1991; Sarver et al., 1990). Recently, it was reported that ribozymes elicited genetic changes in some cells lines to which they were applied; the altered genes included the oncogenes H-ras, c-fos and genes of HIV. Most of this work involved the modification of a target mRNA, based on a specific mutant codon that is cleaved by a specific ribozyme.

5. RNA and RNA Interference

In certain embodiments, the nucleic acid is an RNA molecule. For example, the RNA molecule can be a messenger RNA (mRNA) molecule. In other embodiments, the RNA molecule is an interfering RNA. RNA interference (RNA₁) is a form of gene silencing triggered by double-stranded RNA (dsRNA). DsRNA activates post-transcriptional gene expression surveillance mechanisms that appear to function to defend cells from virus infection and transposon activity. Fire et al. (1998); Grishok et al. (2000); Ketting et al. (1999); Lin & Avery (1999); Montgomery et al. (1998); Sharp (1999); Sharp & Zamore (2000); Tabara et al. (1999). Activation of these mechanisms targets mature, dsRNA-complementary mRNA for destruction. RNAᵢ offers major experimental advantages for study of gene function. These advantages include a very high specificity, ease of movement across cell membranes, and prolonged down-regulation of the targeted gene. Fire et al. (1998); Grishok et al. (2000); Ketting et al. (1999); Lin & Avery (1999); Montgomery et al. (1998); Sharp (1999); Sharp & Zamore (2000); Tabara et al. (1999). RNAᵢ also is incredibly potent. It has been estimated that only a few copies of dsRNA are required to knock down >95% of targeted gene expression in a cell. Fire et al. (1998). Moreover, dsRNA has been shown to silence genes in a wide range of systems, including plants, protozoans, C. elegans and Drosophila. Grishok et al. (2000); Sharp (1999); Sharp & Zamore (1999).

D. Vectors for Cloning, Gene Transfer and Expression

Within certain embodiments, expression vectors are employed to express a polypeptide selected from the group consisting of SEQ ID NOS: 1, 3, 5, 7, 9, 11, 13, 15, 17, 19, 21, 23, 25, 27, 31, 33, 35, 37, 39, 41, 43, 45, and 47. In other embodiments, the expression vectors are used in gene therapy. Expression requires that appropriate signals be provided in the vectors, and which include various regulatory elements, such as enhancers/promoters from both viral and mammalian sources that drive expression of the genes of interest in host cells. Elements designed to optimize messenger RNA stability and translatability in host cells also are defined. The conditions for the use of a number of dominant drug selection markers for establishing permanent, stable cell clones expressing the products are also provided, as is an element that links expression of the drug selection markers to expression of the polypeptide.

1. Regulatory Elements

Throughout this application, the term “expression construct” is meant to include any type of genetic construct containing a nucleic acid coding for a gene product in which part or all of the nucleic acid encoding sequence is capable of being transcribed. The transcript may be translated into a protein, but it need not be. In certain embodiments, expression includes both transcription of a gene and translation of mRNA into a gene product. In other embodiments, expression only includes transcription of the nucleic acid encoding a gene of interest.

In preferred embodiments, the nucleic acid encoding a gene product is under transcriptional control of a promoter. A “promoter” refers to a DNA sequence recognized by the synthetic machinery of the cell, or introduced synthetic machinery, required to initiate the specific transcription of a gene. The phrase “under transcriptional control” means that the promoter is in the correct location and orientation in relation to the nucleic acid to control RNA polymerase initiation and expression of the gene.

The term promoter will be used here to refer to a group of transcriptional control modules that are clustered around the initiation site for RNA polymerase II. Much of the thinking about how promoters are organized derives from analyses of several viral promoters, including those for the HSV thymidine kinase (tk) and SV40 early transcription units. These studies, augmented by more recent work, have shown that promoters are composed of discrete functional modules, each consisting of approximately 7-20 bp of DNA, and containing one or more recognition sites for transcriptional activator or repressor proteins.

At least one module in each promoter functions to position the start site for RNA synthesis. The best known example of this is the TATA box, but in some promoters lacking a TATA box, such as the promoter for the mammalian terminal deoxynucleotidyl transferase gene and the promoter for the SV40 late genes, a discrete element overlying the start site itself helps to fix the place of initiation.

Additional promoter elements regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the tk promoter, the spacing between promoter elements can be increased to 50 bp apart before activity begins to decline. Depending on the promoter, it appears that individual elements can function either co-operatively or independently to activate transcription.

In various embodiments, the human cytomegalovirus (CMV) immediate early gene promoter, the SV40 early promoter, the Rous sarcoma virus long terminal repeat, rat insulin promoter and glyceraldehyde-3-phosphate dehydrogenase can be used to obtain high-level expression of the coding sequence of interest. The use of other viral or mammalian cellular or bacterial phage promoters which are well-known in the art to achieve expression of a coding sequence of interest is contemplated as well, provided that the levels of expression are sufficient for a given purpose.

By employing a promoter with well-known properties, the level and pattern of expression of the protein of interest following transfection or transformation can be optimized. Further, selection of a promoter that is regulated in response to specific physiologic signals can permit inducible expression of the gene product. Tables 2 and 3 list several regulatory elements that may be employed, in the context of the present invention, to regulate the expression of the gene of interest. This list is not intended to be exhaustive of all the possible elements involved in the promotion of gene expression but, merely, to be exemplary thereof.

Enhancers are genetic elements that increase transcription from a promoter located at a distant position on the same molecule of DNA. Enhancers are organized much like promoters. That is, they are composed of many individual elements, each of which binds to one or more transcriptional proteins.

The basic distinction between enhancers and promoters is operational. An enhancer region as a whole must be able to stimulate transcription at a distance; this need not be true of a promoter region or its component elements. On the other hand, a promoter must have one or more elements that direct initiation of RNA synthesis at a particular site and in a particular orientation, whereas enhancers lack these specificities. Promoters and enhancers are often overlapping and contiguous, often seeming to have a very similar modular organization.

Below is a list of viral promoters, cellular promoters/enhancers and inducible promoters/enhancers that could be used in combination with the nucleic acid encoding a gene of interest in an expression construct (Table 2 and Table 3). Additionally, any other promoter/enhancer combination (for example, as per the Eukaryotic Promoter Data Base EPDB) could also be used to drive expression of the gene. Eukaryotic cells can support cytoplasmic transcription from certain bacterial promoters if the appropriate bacterial polymerase is provided, either as part of the delivery complex or as an additional genetic expression construct.

TABLE 3
Promoter and/or Enhancer

| Promoter/Enhancer | References |
|---|---|
| Immunoglobulin Heavy Chain | Banerji et al., 1983; Gilles et al., 1983; Grosschedl |
| | et al., 1985; Atchinson et al., 1986, 1987; Imler et |
| | al., 1987; Weinberger et al., 1984; Kiledjian et al., |
| | 1988; Porton et al.; 1990 |
| Immunoglobulin Light Chain | Queen et al., 1983; Picard et al., 1984 |
| T-Cell Receptor | Luria et al., 1987; Winoto et al., 1989; Redondo et |
| | al.; 1990 |
| HLA DQ a and/or DQ β | Sullivan et al., 1987 |
| β-Interferon | Goodbourn et al., 1986; Fujita et al., 1987; |
| | Goodbourn et al., 1988 |
| Interleukin-2 | Greene et al., 1989 |
| Interleukin-2 Receptor | Greene et al., 1989; Lin et al., 1990 |
| MHC Class II 5 | Koch et al., 1989 |
| MHC Class II HLA-DRa | Sherman et al., 1989 |
| β-Actin | Kawamoto et al., 1988; Ng et al.; 1989 |
| Muscle Creatine Kinase (MCK) | Jaynes et al., 1988; Horlick et al., 1989; Johnson et |
| | al., 1989 |
| Prealbumin (Transthyretin) | Costa et al., 1988 |
| Elastase I | Ornitz et al., 1987 |
| Metallothionein (MTII) | Karin et al., 1987; Culotta et al, 1989 |
| Collagenase | Pinkert et al., 1987; Angel et al., 1987a |
| Albumin | Pinkert et al., 1987; Tronche et al., 1989, 1990 |
| α-Fetoprotein | Godbout et al., 1988; Campere et al., 1989 |
| t-Globin | Bodine et al., 1987; Perez-Stable et al., 1990 |
| β-Globin | Trudel et al., 1987 |
| c-fos | Cohen et al., 1987 |
| c-HA-ras | Triesman, 1986; Deschamps et al., 1985 |
| Insulin | Edlund et al., 1985 |
| Neural Cell Adhesion Molecule | Hirsh et al., 1990 |
| (NCAM) | |
| α₁-Antitrypain | Latimer et al., 1990 |
| H2B (TH2B) Histone | Hwang et al., 1990 |
| Mouse and/or Type I Collagen | Ripe et al., 1989 |
| Glucose-Regulated Proteins | Chang et al., 1989 |
| (GRP94 and GRP78) | |
| Rat Growth Hormone | Larsen et al., 1986 |
| Human Serum Amyloid A (SAA) | Edbrooke et al., 1989 |
| Troponin I (TN I) | Yutzey et al., 1989 |
| Platelet-Derived Growth Factor | Pech et al., 1989 |
| (PDGF) | |
| Duchenne Muscular Dystrophy | Klamut et al., 1990 |
| SV40 | Banerji et al., 1981; Moreau et al., 1981; Sleigh et |
| | al., 1985; Firak et al., 1986; Herr et al., 1986; |
| | Imbra et al., 1986; Kadesch et al., 1986; Wang et |
| | al., 1986; Ondek et al., 1987; Kuhl et al., 1987; |
| | Schaffner et al., 1988 |
| Polyoma | Swartzendruber et al., 1975; Vasseur et al., 1980; |
| | Katinka et al., 1980, 1981; Tyndell et al., 1981; |
| | Dandolo et al., 1983; de Villiers et al., 1984; Hen |
| | et al., 1986; Satake et al., 1988; Campbell and/or |
| | Villarreal, 1988 |
| Retroviruses | Kriegler et al., 1982, 1983; Levinson et al., 1982; |
| | Kriegler et al., 1983, 1984a, b, 1988; Bosze et al., |
| | 1986; Miksicek et al., 1986; Celander et al., 1987; |
| | Thiesen et al., 1988; Celander et al., 1988; Choi et |
| | al., 1988; Reisman et al., 1989 |
| Papilloma Virus | Campo et al., 1983; Lusky et al., 1983; Spandidos |
| | and/or Wilkie, 1983; Spalholz et al., 1985; Lusky |
| | et al., 1986; Cripe et al., 1987; Gloss et al., 1987; |
| | Hirochika et al., 1987; Stephens et al., 1987; Glue |
| | et al., 1988 |
| Hepatitis B Virus | Bulla et al., 1986; Jameel et al., 1986; Shaul et al., |
| | 1987; Spandau et al., 1988; Vannice et al, 1988 |
| Human Immunodeficiency Virus | Muesing et al., 1987; Hauber et al., 1988; |
| | Jakobovits et al., 1988; Feng et al., 1988; Takebe |
| | et al., 1988; Rosen et al., 1988; Berkhout et al., |
| | 1989; Laspia et al., 1989; Sharp et al., 1989; |
| | Braddock et al., 1989 |
| Cytomegalovirus (CMV) | Weber et al., 1984; Boshart et al., 1985; Foecking |
| | et al., 1986 |
| Gibbon Ape Leukemia Virus | Holbrook et al., 1987; Quinn et al., 1989 |

TABLE 4
Inducible Elements

| Element | Inducer | References |
|---|---|---|
| MT II | Phorbol Ester (TFA) | Palmiter et al., 1982; |
| | Heavy metals | Haslinger et al., 1985; |
| | | Searle et al., 1985; Stuart |
| | | et al., 1985; Imagawa et |
| | | al., 1987, Karin et al., |
| | | 1987; Angel et al., 1987b; |
| | | McNeall et al., 1989 |
| MMTV (mouse | Glucocorticoids | Huang et al., 1991; Lee et |
| mammary tumor | | al., 1981; Majors et al., |
| virus) | | 1983; Chandler et al., |
| | | 1983; Lee et al., 1984; |
| | | Ponta et al., 1985; Sakai et |
| | | al., 1988 |
| β-Interferon | poly(rI)x | Tavernier et al., 1983 |
| | poly(rc) | |
| Adenovirus 5 E2 | ElA | Imperiale et al., 1984 |
| Collagenase | Phorbol Ester (TPA) | Angel et al., 1987a |
| Stromelysin | Phorbol Ester (TPA) | Angel et al., 1987b |
| SV40 | Phorbol Ester (TPA) | Angel et al., 1987b |
| Murine MX Gene | Interferon, Newcastle | Hug et al., 1988 |
| | Disease Virus | |
| GRP78 Gene | A23187 | Resendez et al., 1988 |
| α-2-Macroglobulin | IL-6 | Kunz et al., 1989 |
| Vimentin | Serum | Rittling et al., 1989 |
| MHC Class I | Interferon | Blanar et al., 1989 |
| Gene H-2 κb | | |
| HSP70 | ElA, SV40 Large T | Taylor et al., 1989, 1990a, |
| | Antigen | 1990b |
| Proliferin | Phorbol Ester-TPA | Mordacq et al., 1989 |
| Tumor Necrosis | PMA | Hensel et al., 1989 |
| Factor | | |
| Thyroid Stimulating | Thyroid Hormone | Chatterjee et al., 1989 |
| Hormone α Gene | | |

Where a cDNA insert is employed, one will typically desire to include a polyadenylation signal to effect proper polyadenylation of the gene transcript. The nature of the polyadenylation signal is not believed to be crucial to the successful practice of the invention, and any such sequence may be employed such as human growth hormone and SV40 polyadenylation signals. Also contemplated as an element of the expression cassette is a terminator. These elements can serve to enhance message levels and to minimize read through from the cassette into other sequences.

2. Selectable Markers

In certain embodiments of the invention, the cells contain nucleic acid constructs of the present invention, a cell may be identified in vitro or in vivo by including a marker in the expression construct. Such markers would confer an identifiable change to the cell permitting easy identification of cells containing the expression construct. Usually the inclusion of a drug selection marker aids in cloning and in the selection of transformants, for example, genes that confer resistance to neomycin, puromycin, hygromycin, DHFR, GPT, zeocin and histidinol are useful selectable markers. Alternatively, enzymes such as herpes simplex virus thymidine kinase (tk) or chloramphenicol acetyltransferase (CAT) may be employed. Immunologic markers also can be employed. The selectable marker employed is not believed to be important, so long as it is capable of being expressed simultaneously with the nucleic acid encoding a gene product. Further examples of selectable markers are well known to one of skill in the art.

3. Multigene Constructs and IRES

In certain embodiments of the invention, the use of internal ribosome binding sites (IRES) elements are used to create multigene, or polycistronic, messages. IRES elements are able to bypass the ribosome scanning model of 5′ methylated Cap dependent translation and begin translation at internal sites (Pelletier and Sonenberg, 1988). IRES elements from two members of the picornaovirus family (polio and encephalomyocarditis) have been described (Pelletier and Sonenberg, 1988), as well an IRES from a mammalian message (Macejak and Sarnow, 1991). IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple genes can be efficiently expressed using a single promoter/enhancer to transcribe a single message.

Any heterologous open reading frame can be linked to IRES elements. This includes genes for secreted proteins, multi-subunit proteins, encoded by independent genes, intracellular or membrane-bound proteins and selectable markers. In this way, expression of several proteins can be simultaneously engineered into a cell with a single construct and a single selectable marker.

4. Gene Transfer

There are a number of ways in which nucleic acids may be introduced into cells. In certain embodiments of the invention, a vector (also referred to herein as a gene delivery vector) is employed to deliver the expression construct. By way of illustration, in some embodiments, a vector may comprises a virus or a non-viral engineered construct derived.

Viral Gene Transfer.

The ability of certain viruses to enter cells via receptor-mediated endocytosis, to integrate into host cell genome and express viral genes stably and efficiently have made them attractive candidates for the transfer of foreign genes into mammalian cells (Ridgeway, 1988; Nicolas and Rubenstein, 1988; Baichwal and Sugden, 1986; Temin, 1986). The first viruses used as gene delivery vectors were DNA viruses including the papovaviruses (simian virus 40, bovine papilloma virus, and polyoma) (Ridgeway, 1988; Baichwal and Sugden, 1986). Generally, these have a relatively low capacity for foreign DNA sequences and have a restricted host spectrum. They can accommodate only up to 8 kb of foreign genetic material but can be readily introduced in a variety of cell lines and laboratory animals (Nicolas and Rubenstein, 1988; Temin, 1986). Where viral vectors are employed to deliver the gene or genes of interest, it is generally preferred that they be replication-defective, for example as known to those of skill in the art and as described further herein below.

One of the preferred methods for in vivo delivery of expression constructs involves the use of an adenovirus expression vector. “Adenovirus expression vector” is meant to include those constructs containing adenovirus sequences sufficient to (a) support packaging of the construct and (b) to express a polynucleotide that has been cloned therein. In this context, expression does not require that the gene product be synthesized.

In preferred embodiments, the expression vector comprises a genetically engineered form of adenovirus. Knowledge of the genetic organization of adenovirus, a 36 kb, linear, double-stranded DNA virus, allows substitution of large pieces of adenoviral DNA with foreign sequences up to 7 kb (Grunhaus and Horwitz, 1992). In contrast to retrovirus, the adenoviral infection of host cells does not result in chromosomal integration because adenoviral DNA can replicate in an episomal manner without potential genotoxicity. Also, adenoviruses are structurally stable, and no genome rearrangement has been detected after extensive amplification. Adenovirus can infect virtually all epithelial cells regardless of their cell cycle stage and are able to infect non-dividing cells such as, for example, cardiomyocytes. So far, adenoviral infection appears to be linked only to mild disease such as acute respiratory disease in humans.

Adenovirus is particularly suitable for use as a gene delivery vector because of its mid-sized genome, ease of manipulation, high titer, wide target cell range and high infectivity. Both ends of the viral genome contain 100-200 base pair inverted repeats (ITRs), which are cis elements necessary for viral DNA replication and packaging. The early (E) and late (L) regions of the genome contain different transcription units that are divided by the onset of viral DNA replication. The E1 region (E1A and E1B) encodes proteins responsible for the regulation of transcription of the viral genome and a few cellular genes. The expression of the E2 region (E2A and E2B) results in the synthesis of the proteins for viral DNA replication. These proteins are involved in DNA replication, late gene expression and host cell shut-off (Renan, 1990). The products of the late genes, including the majority of the viral capsid proteins, are expressed only after significant processing of a single primary transcript issued by the major late promoter (MLP). The MLP, (located at 16.8 m.u.) is particularly efficient during the late phase of infection, and all the mRNA's issued from this promoter possess a 5′-tripartite leader (TPL) sequence which makes them preferred mRNA's for translation.

In a current system, recombinant adenovirus is generated from homologous recombination between shuttle vector and provirus vector. Due to the possible recombination between two proviral vectors, wild-type adenovirus may be generated from this process. Therefore, it is important to minimize this possibility by, for example, reducing or eliminating adnoviral sequence overlaps within the system and/or to isolate a single clone of virus from an individual plaque and examine its genomic structure.

Generation and propagation of the current adenovirus vectors, which are replication deficient, depend on a unique helper cell line, designated 293, which was transformed from human embryonic kidney cells by Ad5 DNA fragments and constitutively expresses E1 proteins (Graham et al., 1977). Since the E3 region is dispensable from the adenovirus genome (Jones and Shenk, 1978), the current adenovirus vectors, with the help of 293 cells, carry foreign DNA in either the E1, the E3 or both regions (Graham and Prevec, 1991). In nature, adenovirus can package approximately 105% of the wild-type genome (Ghosh-Choudhury et al., 1987), providing capacity for about 2 extra kb of DNA. Combined with the approximately 5.5 kb of DNA that is replaceable in the E1 and E3 regions, the maximum capacity of such adenovirus vectors is about 7.5 kb, or about 15% of the total length of the vector. Additionally, modified adenoviral vectors are now available which have an even greater capacity to carry foreign DNA.

Helper cell lines may be derived from human cells such as human embryonic kidney cells, muscle cells, hematopoietic cells or other human embryonic mesenchymal or epithelial cells. Alternatively, the helper cells may be derived from the cells of other mammalian species that are permissive for human adenovirus. Such cells include, e.g., Vero cells or other monkey embryonic mesenchymal or epithelial cells. As stated above, a preferred helper cell line is 293.

Racher et al. (1995) disclosed improved methods for culturing 293 cells and propagating adenovirus. In one format, natural cell aggregates are grown by inoculating individual cells into 1 liter siliconized spinner flasks (Techne, Cambridge, UK) containing 100-200 ml of medium. Following stirring at 40 rpm, the cell viability is estimated with trypsan blue. In another format, Fibra-Cel microcarriers (Bibby Sterlin, Stone, UK) (5 g/l) is employed as follows. A cell inoculum, resuspended in 5 ml of medium, is added to the carrier (50 ml) in a 250 ml Erlenmeyer flask and left stationary, with occasional agitation, for 1 to 4 h. The medium is then replaced with 50 ml of fresh medium and shaking initiated. For virus production, cells are allowed to grow to about 80% confluence, after which time the medium is replaced (to 25% of the final volume) and adenovirus added at an MOI of 0.05. Cultures are left stationary overnight, following which the volume is increased to 100% and shaking commenced for another 72 h.

Other than the requirement that the adenovirus vector be replication defective, or at least conditionally defective, the nature of the adenovirus vector is not believed to be crucial to the successful practice of the invention. The adenovirus may be selected from any of the 42 different known serotypes or subgroups A-F. Adenovirus type 5 of subgroup C is a preferred starting material for obtaining a replication-defective adenovirus vector for use in the present invention. This is, in part, because Adenovirus type 5 is a human adenovirus about which a great deal of biochemical and genetic information is known, and it has historically been used for most constructions employing adenovirus as a vector.

As stated above, one adenoviral vector according to the present invention lacks an adenovirus E1 region and thus, is replication. Typically, it is most convenient to introduce the polynucleotide encoding the gene of interest at the position from which the E1-coding sequences have been removed. However, the position of insertion of the construct within the adenovirus sequences is not critical to the invention. Further, other adenoviral sequences may be deleted and/or inactivated in addition to or in lieu of the E1 region. For example, the E2 and E4 regions are both necessary for adenoviral replication and thus may be modified to render an adenovirus vector replication-defective, in which case a helper cell line or helper virus complex may employed to provide such deleted/inactivated genes in trans. The polynucleotide encoding the gene of interest may alternatively be inserted in lieu of a deleted E3 region such as in E3 replacement vectors as described by Karlsson et al. (1986), or in a deleted E4 region where a helper cell line or helper virus complements the E4 defect. Other modifications are known to those of skill in the art and are likewise contemplated herein.

Adenovirus is easy to grow and manipulate and exhibits broad host range in vitro and in vivo. This group of viruses can be obtained in high titers, e.g., 10⁹-10¹² plaque-forming units per ml, and they are highly infective. The life cycle of adenovirus does not require integration into the host cell genome. The foreign genes delivered by adenovirus vectors are episomal and, therefore, have low genotoxicity to host cells. No side effects have been reported in studies of vaccination with wild-type adenovirus (Couch et al., 1963; Top et al., 1971), demonstrating their safety and therapeutic potential as in vivo gene transfer vectors.

Adenovirus vectors have been used in eukaryotic gene expression (Levrero et al., 1991; Gomez-Foix et al., 1992) and vaccine development (Grunhaus and Horwitz, 1992; Graham and Prevec, 1992). Recently, animal studies indicated that recombinant adenovirus could be used for gene therapy (Stratford-Perricaudet and Perricaudet, 1991; Stratford-Perricaudet et al., 1990; Rich et al., 1993). Studies in administering recombinant adenovirus to different tissues include administration via intracoronary catheter into one or more coronary arteries of the heart (Hammond, et al., U.S. Pat. Nos. 5,792,453 and 6,100,242) trachea instillation (Rosenfeld et al., 1991; Rosenfeld et al., 1992), muscle injection (Ragot et al., 1993), peripheral intravenous injections (Herz and Gerard, 1993) and stereotactic inoculation into the brain (Le Gal La Salle et al., 1993).

The retroviruses are a group of single-stranded RNA viruses characterized by an ability to convert their RNA to double-stranded DNA in infected cells by a process of reverse-transcription (Coffin, 1990). The resulting DNA then stably integrates into cellular chromosomes as a provirus and directs synthesis of viral proteins. The integration results in the retention of the viral gene sequences in the recipient cell and its descendants. The retroviral genome contains three genes, gag, pol, and env that code for capsid proteins, polymerase enzyme, and envelope components, respectively. A sequence found upstream from the gag gene contains a signal for packaging of the genome into virions. Two long terminal repeat (LTR) sequences are present at the 5′ and 3′ ends of the viral genome. These contain strong promoter and enhancer sequences and are also required for integration in the host cell genome (Coffin, 1990).

In order to construct a retroviral vector, a nucleic acid encoding a gene of interest is inserted into the viral genome in the place of certain viral sequences to produce a virus that is replication-defective. In order to produce virions, a packaging cell line containing the gag, pol, and env genes but without the LTR and packaging components is constructed (Mann et al., 1983). When a recombinant plasmid containing a cDNA, together with the retroviral LTR and packaging sequences is introduced into this cell line (by calcium phosphate precipitation for example), the packaging sequence allows the RNA transcript of the recombinant plasmid to be packaged into viral particles, which are then secreted into the culture media (Nicolas and Rubenstein, 1988; Temin, 1986; Mann et al., 1983). The media containing the recombinant retroviruses is then collected, optionally concentrated, and used for gene transfer. Retroviral vectors are able to infect a broad variety of cell types. However, integration and stable expression require the division of host cells (Paskind et al., 1975).

A novel approach designed to allow specific targeting of retrovirus vectors was recently developed based on the chemical modification of a retrovirus by the chemical addition of lactose residues to the viral envelope. This modification could permit the specific infection of hepatocytes via sialoglycoprotein receptors.

A different approach to targeting of recombinant retroviruses was designed in which biotinylated antibodies against a retroviral envelope protein and against a specific cell receptor were used. The antibodies were coupled via the biotin components by using streptavidin (Roux et al., 1989). Using antibodies against major histocompatibility complex class I and class II antigens, they demonstrated the infection of a variety of human cells that bore those surface antigens with an ecotropic virus in vitro (Roux et al., 1989).

There are certain limitations to the use of retrovirus vectors in all aspects of the present invention. For example, retrovirus vectors usually integrate into random sites in the cell genome. This can lead to insertional mutagenesis through the interruption of host genes or through the insertion of viral regulatory sequences that can interfere with the function of flanking genes (Varmus et al., 1981). Another concern with the use of defective retrovirus vectors is the potential appearance of wild-type replication-competent virus in the packaging cells. This can result from recombination events in which the intact-sequence from the recombinant virus inserts upstream from the gag, pol, env sequence integrated in the host cell genome. However, new packaging cell lines are now available that should greatly decrease the likelihood of recombination (Markowitz et al., 1988; Hersdorffer et al., 1990).

Other viral vectors may be employed as expression constructs in the present invention. Vectors derived from viruses such as vaccinia virus (Ridgeway, 1988; Baichwal and Sugden, 1986; Coupar et al., 1988) adeno-associated virus (AAV) (Ridgeway, 1988; Baichwal and Sugden, 1986; Hermonat and Muzycska, 1984) and herpesviruses may be employed. They offer several attractive features for various mammalian cells (Friedmann, 1989; Ridgeway, 1988; Baichwal and Sugden, 1986; Coupar et al., 1988; Horwich et al., 1990).

With the recent recognition of defective hepatitis B viruses, new insight was gained into the structure-function relationship of different viral sequences. In vitro studies showed that the virus could retain the ability for helper-dependent packaging and reverse transcription despite the deletion of up to 80% of its genome (Horwich et al., 1990). This indicated that large portions of the genome could be replaced with foreign genetic material. The hepatotropism and persistence (integration) were particularly attractive properties for liver-directed gene transfer. Chang et al., recently introduced the chloramphenicol acetyltransferase (CAT) gene into duck hepatitis B virus genome in the place of the polymerase, surface, and pre-surface coding sequences. It was co-transfected with wild-type virus into an avian hepatoma cell line. Culture media containing high titers of the recombinant virus were used to infect primary duckling hepatocytes. Stable CAT gene expression was detected for at least 24 days after transfection (Chang et al., 1991).

Non-Viral Delivery.

Several non-viral gene delivery vectors for the transfer of expression constructs into mammalian cells also are contemplated by the present invention. These include calcium phosphate precipitation (Graham and Van Der Eb, 1973; Chen and Okayama, 1987; Rippe et al., 1990) DEAE-dextran (Gopal, 1985), electroporation (Tur-Kaspa et al., 1986; Potter et al., 1984), direct microinjection (Harland and Weintraub, 1985), DNA-loaded liposomes (Nicolau and Sene, 1982; Fraley et al., 1979) and lipofectamine-DNA complexes, cell sonication (Fechheimer et al., 1987), gene bombardment using high velocity microprojectiles (Yang et al., 1990), and receptor-mediated transfection (Wu and Wu, 1987; Wu and Wu, 1988). Some of these techniques may be successfully adapted for in vivo or ex vivo use.

In yet another embodiment, the expression vector may simply consist of naked recombinant DNA or plasmids comprising the expression construct. Transfer of the construct may be performed by any of the methods mentioned above which physically or chemically permeabilize the cell membrane. This is particularly applicable for transfer in vitro but it may be applied to in vivo use as well. Dubensky et al. (1984) successfully injected polyomavirus DNA in the form of calcium phosphate precipitates into liver and spleen of adult and newborn mice demonstrating active viral replication and acute infection. Benvenisty and Neshif (1986) also demonstrated that direct intraperitoneal injection of calcium phosphate-precipitated plasmids results in expression of the transfected genes. It is envisioned that DNA encoding a gene of interest may also be transferred in a similar manner in vivo and express the gene product.

In still another embodiment of the invention, transferring of a naked DNA expression construct into cells may involve particle bombardment. This method depends on the ability to accelerate DNA-coated microprojectiles to a high velocity allowing them to pierce cell membranes and enter cells without killing them (Klein et al., 1987). Several devices for accelerating small particles have been developed. One such device relies on a high voltage discharge to generate an electrical current, which in turn provides the motive force (Yang et al., 1990). The microprojectiles used have consisted of biologically inert substances such as tungsten or gold beads.

Selected organs including the liver, skin, and muscle tissue of rats and mice have been bombarded in vivo (Yang et al., 1990; Zelenin et al., 1991). This may require surgical exposure of the tissue or cells, to eliminate any intervening tissue between the gun and the target organ, i.e., ex vivo treatment. Again, DNA encoding a particular gene may be delivered via this method and still be incorporated by the present invention.

In a further embodiment of the invention, the expression construct may be entrapped in a liposome, another non-viral gene delivery vector. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh and Bachhawat, 1991). Also contemplated are lipofectamine-DNA complexes.

Liposome-mediated nucleic acid delivery and expression of foreign DNA in vitro has been very successful. Wong et al., (1980) demonstrated the feasibility of liposome-mediated delivery and expression of foreign DNA in cultured chick embryo, HeLa and hepatoma cells. Nicolau et al. (1987) accomplished successful liposome-mediated gene transfer in rats after intravenous injection.

In certain embodiments of the invention, the liposome may be complexed with a hemagglutinating virus (HVJ). This has been shown to facilitate fusion with the cell membrane and promote cell entry of liposome-encapsulated DNA (Kaneda et al., 1989). In other embodiments, the liposome may be complexed or employed in conjunction with nuclear non-histone chromosomal proteins (HMG-1) (Kato et al., 1991). In yet further embodiments, the liposome may be complexed or employed in conjunction with both HVJ and HMG-1. In that such expression constructs have been successfully employed in transfer and expression of nucleic acid in vitro and in vivo, then they are applicable for the present invention. Where a bacterial promoter is employed in the DNA construct, it also will be desirable to include within the liposome an appropriate bacterial polymerase.

Other expression constructs which can be employed to deliver a nucleic acid encoding a particular gene into cells are receptor-mediated delivery vehicles. These take advantage of the selective uptake of macromolecules by receptor-mediated endocytosis in almost all eukaryotic cells. Because of the cell type-specific distribution of various receptors, the delivery can be highly specific (Wu and Wu, 1993).

Receptor-mediated gene targeting vehicles generally consist of two components: a cell receptor-specific ligand and a DNA-binding agent. Several ligands have been used for receptor-mediated gene transfer. The most extensively characterized ligands are asialoorosomucoid (ASOR) (Wu and Wu, 1987) and transferrin (Wagner et al., 1990). Recently, a synthetic neoglycoprotein, which recognizes the same receptor as ASOR, has been used as a gene delivery vehicle (Ferkol et al., 1993; Perales et al., 1994) and epidermal growth factor (EGF) has also been used to deliver genes to squamous carcinoma cells (Myers, EPO 0 273 085).

In other embodiments, the delivery vehicle may comprise a ligand and a liposome. For example, Nicolau et al. (1987) employed lactosyl-ceramide, a galactose-terminal asialganglioside, incorporated into liposomes and observed an increase in the uptake of the insulin gene by hepatocytes. Thus, it is feasible that a nucleic acid encoding a particular gene also may be specifically delivered into a cell type by any number of receptor-ligand systems with or without liposomes. For example, epidermal growth factor (EGF) may be used as the receptor for mediated delivery of a nucleic acid into cells that exhibit upregulation of EGF receptor. Mannose can be used to target the mannose receptor on liver cells. Also, antibodies to CD5 (CLL), CD22 (lymphoma), CD25 (T-cell leukemia) and MAA (melanoma) can similarly be used as targeting moieties.

In certain embodiments, gene transfer may more easily be performed under ex vivo conditions. Ex vivo gene therapy refers to the isolation of cells from an animal, the delivery of a nucleic acid into the cells in vitro, and then the return of the modified cells back into an animal. This may involve the surgical removal of tissue/organs from an animal or the primary culture of cells and tissues.

E. Screening Methods

The present invention includes embodiments that provide for methods of screening for a sleep-altering composition. Virtually any assay technique known to those of skill in the art is contemplated by the present invention. For example, the assays may comprise random high-throughput screening of large libraries of candidate substances. Alternatively, the assays may be used to focus on particular classes of compounds selected with an eye towards structural attributes that are believed to make them more likely to alter expression level or activity of a gene of interest. The assays involved in these screening methods may include cell-free assays, in vitro assays, in cyto assays, in vivo assays, or any assay technique known to those of skill in the art.

A sleep-altering composition is any composition that can modify the sleep requirements or the response to sleep deprivation of a subject. The candidate substance to be tested can be a substance suspected of altering expression level or activity of CG18190, Jheh 1, CG7228, lama, disco, CG6664, Casein kinase II β subunit, CG9171, GstE1, cAMP-dependent protein kinase R2, CG 5161, MESR3, Meics, Atpalpha, Calx, Rlip, nompC, H15, Lam, Glu-RIIA, Glu-RIIB, Ork1, Shaker, or Hyperkinetic gene products, or a homolog thereof. Alternatively, the candidate may simply be a member of a selected library of compounds. It will, of course, be understood that all the screening methods of the present invention are useful in themselves notwithstanding the fact that effective candidates may not be found. The invention provides methods for screening for such candidates, not solely methods of finding them.

1. Modulators

A modulator may be a protein or fragment thereof, a small molecule, an antibody, an oligonucleotide, or even a polynucleotide. It may prove to be the case that the most useful pharmacological compounds will be compounds that are structurally related to known modulators of the expression level or activity of CG18190, Jheh 1, CG7228, lama, disco, CG6664, Casein kinase II β subunit, CG9171, GstE1, cAMP-dependent protein kinase R2 , CG15161, MESR3, Meics, Atpalpha, Calx, Rlip, nompC, H15, Lam, Glu-RIIA, Glu-RIIB, Ork1, Shaker, or Hyperkinetic gene products. Using lead compounds to help develop improved compounds is known as “rational drug design” and includes not only comparisons with known modulators, but predictions relating to the structure of target molecules.

The goal of rational drug design is to produce structural analogs of biologically active polypeptides or target compounds. By creating such analogs, it is possible to fashion drugs, which are more active or stable than the natural molecules, which have different susceptibility to alteration or which may affect the function of various other molecules. In one approach, one would generate a three-dimensional structure for a target molecule, or a fragment thereof. This could be accomplished by x-ray crystallography, computer modeling or by a combination of both approaches.

It also is possible to use antibodies to ascertain the structure of a target compound activator or inhibitor. In principle, this approach yields a pharmacore upon which subsequent drug design can be based. It is possible to bypass protein crystallography altogether by generating anti-idiotypic antibodies to a functional, pharmacologically active antibody. As a mirror image of a mirror image, the binding site of anti-idiotype would be expected to be an analog of the original antigen. The anti-idiotype could then be used to identify and isolate peptides from banks of chemically- or biologically-produced peptides. Selected peptides would then serve as the pharmacore. Anti-idiotypes may be generated using the methods described herein for producing antibodies, using an antibody as the antigen.

On the other hand, one may simply acquire, from various commercial sources, small molecule libraries that are believed to meet the basic criteria for useful drugs in an effort to “brute force” the identification of useful compounds. Screening of such libraries, including combinatorially generated libraries (e.g., peptide libraries), is a rapid and efficient way to screen large number of related (and unrelated) compounds for activity. Combinatorial approaches also lend themselves to rapid evolution of potential drugs by the creation of second, third and fourth generation compounds modeled of active, but otherwise undesirable compounds.

Candidate compounds may include compounds isolated from natural sources, such as animals, bacteria, fungi, plant sources, including leaves and bark, and marine samples may be assayed as candidates for the presence of potentially useful pharmaceutical agents. It will be understood that the pharmaceutical agents to be screened could also be derived or synthesized from chemical compositions or man-made compounds. Thus, it is understood that the candidate substance identified by the present invention may be peptide, polypeptide, oligonucleotide, polynucleotide, small molecule inhibitors or any other compounds that may be designed through rational drug design starting from known inhibitors or stimulators.

Other suitable modulators include antisense molecules, ribozymes, small interfering RNAs, and antibodies (including single-chain antibodies or expression constructs coding thereof), each of which would be specific for a given target molecule. Such compounds are described in greater detail elsewhere in this document. For example, an antisense molecule that bound to a translational or transcriptional start site, or splice junctions, would be an ideal candidate inhibitor.

In addition to the modulating compounds initially identified, the inventors also contemplate that other sterically similar compounds may be formulated to mimic the key portions of the structure of the modulators. Such compounds, which may include peptidomimetics of peptide modulators, may be used in the same manner as the initial modulators.

An agent that alters sleep may, according to the present invention, be one which exerts its effect upstream, downstream or directly on a known pathway involved in sleep regulation. Regardless of the type of composition identified by the present screening methods, the effect of the composition is sleep alteration.

2. In Vitro Assays

A quick, inexpensive and easy assay to run is an in vitro assay. Such assays can be run quickly and in large numbers, thereby increasing the amount of information obtainable in a short period of time. A variety of vessels may be used to run the assays, including test tubes, plates, dishes and other surfaces such as dipsticks or beads. One example of a cell free assay is a binding assay. While not directly addressing effects on the activity of a molecule, much less sleep alteration, the ability of a candidate substance to bind to a target in vitro may be evidence of a related biological effect on an organism. For example, binding of a molecule to a target may, in and of itself, be inhibitory, due to steric, allosteric or charge-charge interactions.

The target may be either free in solution, fixed to a support, expressed in or on the surface of a cell. Either the target or the compound may be labeled, thereby permitting determining of binding. Usually, the target will be the labeled species, decreasing the chance that the labeling will interfere with or enhance binding. Competitive binding formats can be performed in which one of the agents is labeled, and one may measure the amount of free label versus bound label to determine the effect on binding.

A technique for high throughput screening of compounds is described in WO 84/03564, U.S. Pat. No. 6,457,809, U.S. Pat. No. 6,406,921, and U.S. Pat. No. 5,994,131. Large numbers of small peptide test compounds are synthesized on a solid substrate, such as plastic or some other surface. Bound polypeptide is detected by various methods.

3. In Cyto Assays

Various cells and cell lines can be utilized for screening assays, including cells specifically engineered for this purpose. A particularly useful example of a cell for use in the present screening assays is a Drosophila cell of neuronal origin. However, other cells including those from mammals and even humans may be used. One of skill in the art would understand that the invention disclosed herein contemplates a wide variety of in cyto assays for measuring parameters that correlate with expression level or activity of CG18190, Jheh 1, CG7228, lama, disco, CG6664, Casein kinase II β subunit, CG9171, GstE1, cAMP-dependent protein kinase R2, CG15161, MESR3, Meics, Atpalpha, Calx, Rlip, nompC, H15, Lam, Glu-RIIA, Glu-RIIB, Ork1, Shaker, or Hyperkinetic gene products.

Depending on the assay, culture may be required. The cell may be examined using any of a number of different physiologic assays to assess effects, such as phosphorylation levels, enzymatic activity (in case of enzymes), binding properties (in case of receptors), or electrophysiological currents (in case of ionic channels). Alternatively, molecular analysis may be performed, for example, looking at protein expression, mRNA expression (including differential display of whole cell or polyA NA) and other parameters associated with expression level or activity of CG18190, Jheh 1, CG7228, lama, disco, CG6664, Casein kinase II β subunit, CG9171, GstE1, cAMP-dependent protein kinase R2 , CG15161, MESR3, Meics, Atpalpha, Calx, Rlip, nompC, H15, Lam, Glu-RIIA, Glu-RIIB, Ork1, Shaker, or Hyperkinetic gene products.

4. In Vivo Assays

In vivo assays may involve the use of various animal, particularly including flies, but also mammals and humans. Specific assays may also use non-human transgenic animals that have been engineered to have specific defects or carry markers that can be used to measure the ability of a candidate substance to reach and effect different cells within the organism. Due to their size, ease of handling, and information on their physiology and genetic make-up, flies are the preferred transgenic embodiment, with mice being the preferred mammalian transgenics. However, other animals are suitable as well, including rats, rabbits, hamsters, guinea pigs, gerbils, woodchucks, cats, dogs, sheep, goats, pigs, cows, horses and monkeys (including chimps, gibbons and baboons). Assays for modulators may be conducted using an animal model derived from any of these species.

In such assays, one or more candidate substances are administered to the organism, and the ability of the candidate substance(s) to alter one or more characteristics, as compared to a similar animal not treated with the candidate substance(s), identifies an effect of the candidate substance on the expression level or activity of a gene product selected from the group consisting of CG18190, Jheh 1, CG7228, lama, disco, CG6664, Casein kinase II β subunit, CG9171, GstE1, cAMP-dependent protein kinase R2, CG15161, MESR3, Meics, Atpalpha, Calx, Rlip, nompC, H15, Lam, Glu-RIIA, Glu-RIIB, Ork1, Shaker, or Hyperkinetic gene products.

Treatment of organisms with test compounds will involve the administration of the compound, in an appropriate form, to the organism. Any animal model known to those of skill in the art can be used in the screening techniques of the present invention. Administration will be by any route that could be utilized for clinical or non-clinical purposes, including but not limited to oral, nasal, buccal, intratumoral, or even topical. Alternatively, administration may be by intratracheal instillation, bronchial instillation, intradermal, subcutaneous, intramuscular, intraperitoneal. inhalation or intravenous injection. Determining the effectiveness of a compound in vivo may involve a variety of different criteria. Also, measuring toxicity and dose response can be performed in animals in a more meaningful fashion than in in vitro or in cyto assays.

5. Drosophila Assays

Various assays are available for assessing sleep and sleep effects in Drosophila, several of which are disclosed in the examples. Such assays are designed to measure sleep, the effects of sleep deprivation on various performance aspects such as vigilance and memory.

DAMS.

Fly behavior can be monitored using visual observation, an ultrasound activity monitoring system, and an automatic infrared system (Drosophila Activity Monitoring System, DAMS; Trikinetics, Waltham, Mass.). The ultrasound method (Shaw et al., 2000) allows a continuous, high-resolution measurement of the behavior of a single fly housed inside an ultrasound standing wave chamber (FIG. 1A). Whenever the fly moves its head, wings, or limbs, a perturbation of the standing wave is produced and is counted as a movement. Although very precise, this method is impractical for evaluating sleep/waking parameters in a large-scale project. The DAMS is instead designed to monitor hundreds or thousands of flies simultaneously. One DAMS monitor contains 32 glass tubes, each housing a single fly and enough food for 1-week recording (FIG. 1B). As each fly moves back and forth in its tube, it interrupts an infrared light beam that bisects the tube. Each crossing is counted as a movement and the number of movement every minute are summed up and expressed as “activity index”. Both the ultrasound and the infrared system had been validated by visual observation and give similar results: flies are mostly active and moving around during the day, while during the night they show long periods of immobility that can last several hours (FIG. 1C).

Behavioral quiescence qualifies as sleep only if it is accompanied by a reversible increase in arousal threshold. Arousal threshold in flies has been measured using vibratory, visual, auditory stimuli (Shaw et al., 2000; Nitz et al., 2002) and, more recently, thermal stimuli (inventors' unpublished results). In all cases it was found that flies that had been behaviorally awake immediately before the stimulus readily responded to low and medium stimulus intensities. By contrast, flies that had been behaviorally quiescent for 5 min or more rarely showed a motor response, although they quickly responded when the stimulus intensity was increased. Thus, sleep can be operatively defined in flies as any period of behavioral quiescence (no counts detected by the DAMS) lasting longer than 5 minutes (FIG. 1D).

Agitator Platform.

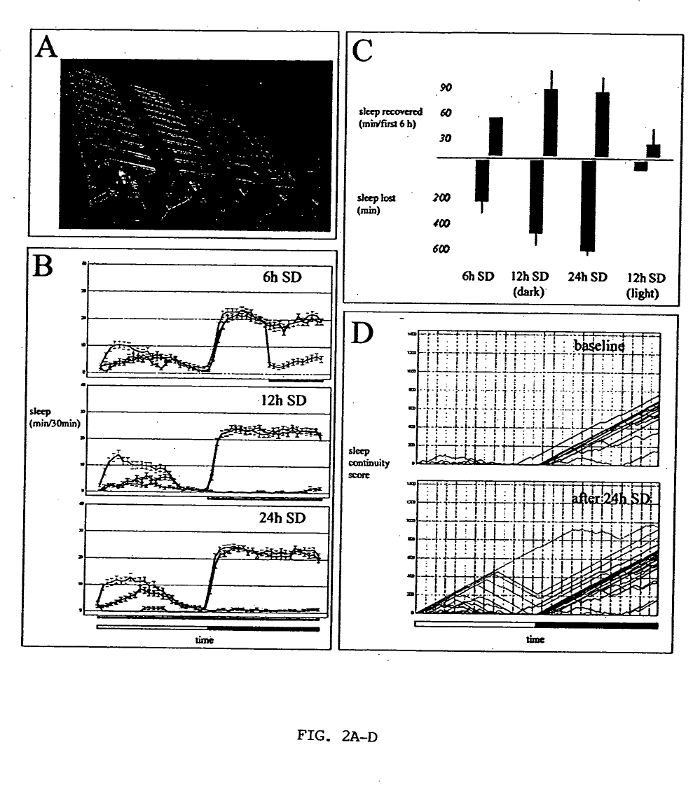

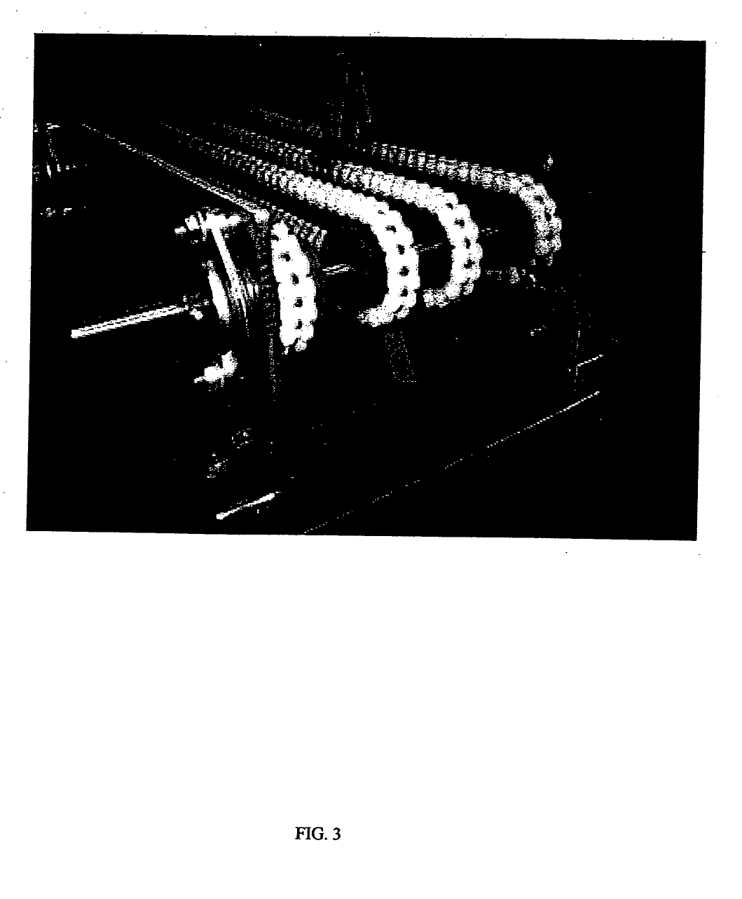

Sleep deprivation can be performed by gentle tapping on the glass tube whenever the fly stops moving for more than 5 min, or automatically. Currently, in the inventors' laboratory, wakefulness is enforced by placing the DAMS monitors vertically within a framed box able to rotate along its major axis under the control of a motor (FIG. 2A). The box can rotate 180° C. clock-wise or counter-clock-wise (2-3 revolutions/min). At the nadir of each rotation, the monitors are dropped 1 cm. This causes the flies to fall from their current position to the bottom of the tube. This method can effectively sleep deprive thousands of flies simultaneously for one or more days. Wild-type flies sleep longer after being sleep deprived (FIGS. 2B-C). Like in mammals, this sleep rebound occurs mainly immediately after the end of the sleep deprivation period (FIG. 2B), is more pronounced after longer (12-24 hours) than after shorter (6 hours) periods of sleep loss, and the recovered sleep only represents a fraction of what was lost (FIG. 2C). Importantly, there is no increase in sleep duration when female flies are subjected to 12 hours of the same stimulation during the day (when they are normally awake), ruling out aspecific effects (FIG. 2C). In mammals, sleep after sleep deprivation is also qualitatively different, i.e., is richer in slow-wave activity, a well-characterized EEG marker of sleep intensity and sleep pressure, and is less fragmented (i.e., there are fewer periods of brief awakenings during sleep; refs. Borbely and Achermann, 1999; Huber et al., 2000). New evidence from the inventors' laboratory shows that in flies sleep continuity is increased and the number of brief awakenings is reduced after sleep deprivation (Huber et al., 2004; FIG. 2D).

VAV Stimulus.

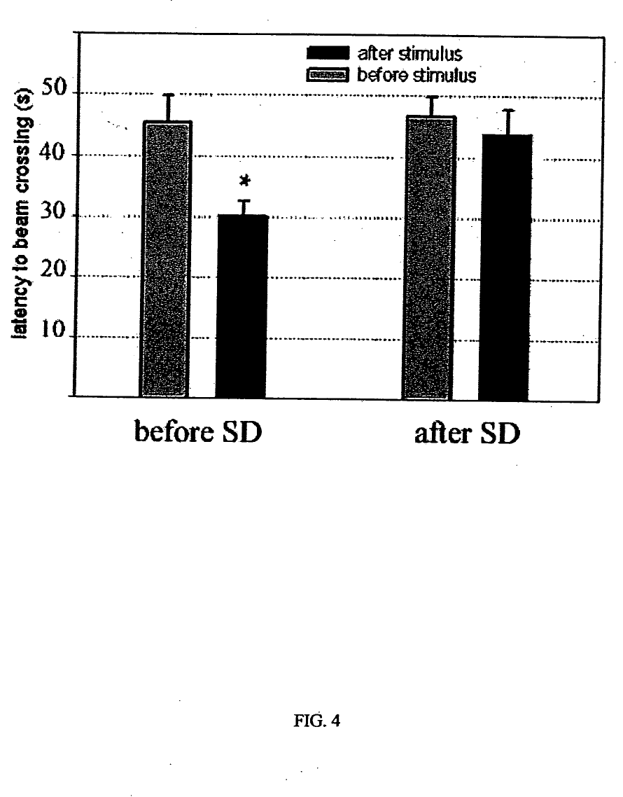

The inventors have assessed the effects of sleep deprivation on vigilance and memory in wild-type flies using vigilance tests and memory tests. In the vigilance test (FIG. 3), the locomotor response induced by a complex stimulus (visual+acoustic+vibratory) produced by a flap vigorously pushed against the glass tubes where the flies are housed is measured. Wild-type flies, as well as most mutant lines tested so far, respond by moving away from the side where the stimulus is delivered. By doing so, they cross the infrared beam, and the latency to crossing is measured by the DAMS monitor. The inventors only consider periods during which flies are awake and spontaneously patrolling the tubes (flies do not respond to the stimulus when asleep). The inventors calculate the mean latency to crossing the infrared beam from the time point at which the stimulus is delivered. For comparison, we then calculate the mean latency to crossing the infrared beam for a time point 1 minute before the stimulus is delivered. The difference before the 2 mean latencies is taken as an indicator of vigilance. A recent study performed in the inventors' laboratory shows that this difference is reduced in wild-type flies after 24 hours of sleep deprivation, an indication that vigilance is affected by sleep loss (Huber et al., 2004; FIG. 4).

Heat Box.

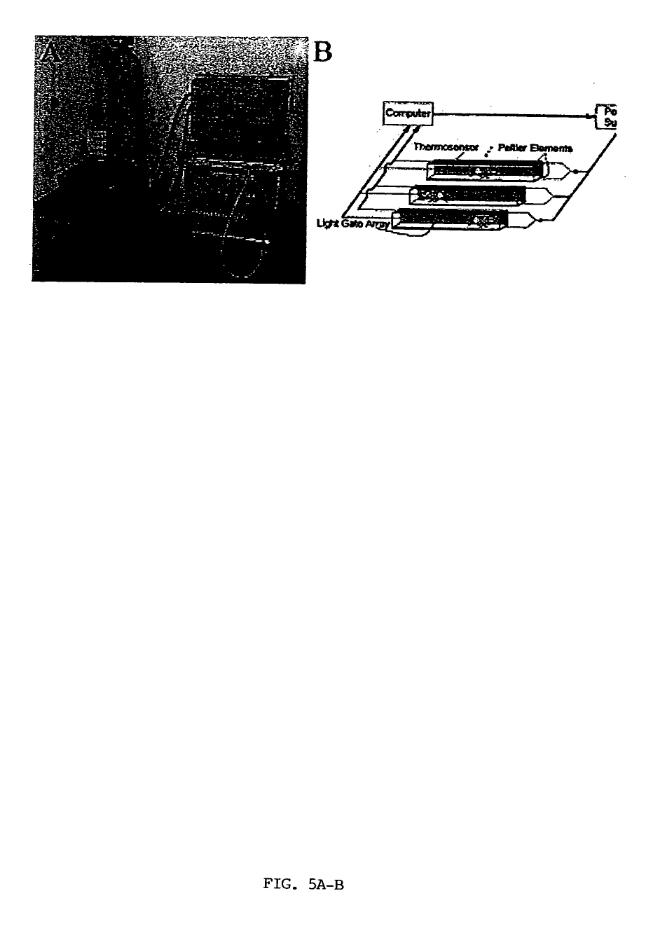

The ability of flies to learn and to retain memories can be tested using the heat box system, introduced by Dr. Martin Heisenberg (Wustmann et al., 1996; Wustmann and Heisenberg, 1997; Putz and Heisenberg, 2002). In each heating chamber of this apparatus (FIG. 5), a fly can be conditioned to avoid one side of the chamber if the chamber is heated whenever the fly enters that side; in a subsequent memory test without heat, the fly keeps avoiding the heat-associated side. The procedure has been extensively tested and offers several advantages relative to other methods: 1) it is fast, robust, requires little handling and therefore it is suitable to test a large number of flies; 2) flies are freely moving; 3) statistically significant learning curves can be obtained for individual flies. The inventors' laboratory has recently acquired a heat box system with 16 individual heating chambers. The system was built in Germany by the same people who developed the system in Dr. Heisenberg's laboratory several years ago.

F. Diagnosing and Treating Sleep Defects

In another aspect of the invention, there inventors now provide methods for identifying defects in the expression and activity of various gene targets that play important roles in sleep and sleep regulation. Thus, in another embodiment, there are provided methods for modulating the need for sleep or sleep deprivation recovery in a subject, include modulating the expression level or activity of one or more gene products encoded by a selected from the group consisting of CG18190, Jheh 1, CG7228, lama, disco, CG6664, Casein kinase II β subunit, CG9171, GstE1, cAMP-dependent protein kinase R2, CG15161, MESR3, Meics, Atpalpha, Calx, Rlip, nompC, H15, Lam, Glu-RIIA, Glu-RIIB, Ork1, shaker and HyperKinetic. In addition, a wide variety of defects in these targets, including point mutations, deletions, rearrangements or insertions in regulatory or coding sequences, as well as increases or decreases in levels of expression, may be assessed using standard technologies, as described below.

1. Genetic Diagnosis

Some embodiments of the instant invention pertain to methods for identifying the basis of a sleep disorder in a subject that involve obtaining mRNA from a neuronal cell of the subject and measuring the expression level of an mRNA selected from CG18190, Jheh 1, CG7228, lama, disco, CG6664, Casein kinase II β subunit, CG9171, GstE1, cAMP-dependent protein kinase R2 , CG15161, MESR3, Meics, Atpalpha, Calx, Rlip, nompC, H15, Lam, Glu-RIIA, Glu-RIIB, Ork1, shaker and Hyperkinetic. Alternatively, this may comprise determining specific alterations in the mRNA or the genomic sequence from which the mRNA derives.

A suitable mRNA-containing biological sample can be a neuronal cell from any tissue of the subject. Various sources include the skin, muscle, facia, brain, prostate, breast, endometrium, lung, head & neck, pancreas, small intestine, blood cells, liver, testes, ovaries, colon, skin, stomach, esophagus, spleen, lymph node, bone marrow or kidney.

Nucleic acid used is isolated from neuronal cells contained in the biological sample, according to standard methodologies (Sambrook et al., 2001). The nucleic acid may be genomic DNA or fractionated or whole cell RNA. Where RNA is used, it may be desired to convert the RNA to a complementary DNA. In one embodiment, the RNA is whole cell RNA; in another, it is poly-A RNA. Normally, the nucleic acid is amplified.

Next, the identified product is detected. Depending on the format, the specific nucleic acid of interest is identified in the sample directly using amplification or with a second, labeled nucleic acid following amplification. In certain applications, the detection may be performed by visual means (e.g., ethidium bromide staining of a gel or integral labeling). Alternatively, the detection may involve indirect identification of the product via chemiluminescence, radioactive scintigraphy of radiolabel or fluorescent label or even via a system using electrical or thermal impulse signals (Affymax Technology; Bellus, 1994).

Various types of defects may be identified by the present methods. Thus, “alterations” should be read as including deletions, insertions, point mutations, rearrangements and duplications. Point mutations result in stop codons, frameshift mutations or amino acid substitutions. Somatic mutations are those occurring in non-germline tissues. Germ-line tissue can occur in any tissue and are inherited. Mutations in and outside the coding region also may affect the amount of protein produced, both by altering the transcription of the gene or in destabilizing or otherwise altering the processing of either the transcript (mRNA) or protein.

A variety of different assays are contemplated in this regard, including but not limited to, fluorescent in situ hybridization (FISH), direct DNA sequencing, PFGE analysis, Southern or Northern blotting, single-stranded conformation analysis (SSCA), RNAse protection assay, allele-specific oligonucleotide (ASO), dot blot analysis, denaturing gradient gel electrophoresis, RFLP and PCRTM-SSCP.

(i) Primers and Probes

The term primer, as defined herein, is meant to encompass any nucleic acid that is capable of priming the synthesis of a nascent nucleic acid in a template-dependent process. Typically, primers are oligonucleotides from ten to twenty base pairs in length, but longer sequences can be employed. Primers may be provided in double-stranded or single-stranded form, although the single-stranded form is preferred. Probes are defined differently, although they may act as primers. Probes, while perhaps capable of priming, are designed to binding to the target DNA or RNA and need not be used in an amplification process.

In preferred embodiments, the probes or primers are labeled with radioactive species (³²P, ¹⁴C, ³⁵S, ³H, or other label), with a fluorophore (rhodamine, fluorescein) or a chemillumiscent (luciferase).

(ii) Template Dependent Amplification Methods

A number of template dependent processes are available to amplify the marker sequences present in a given template sample. One of the best known amplification methods is the polymerase chain reaction (referred to as PCR™) which is described in detail in U.S. Pat. Nos. 4,683,195, 4,683,202 and 4,800,159, and in Innis et al., 1990, each of which is incorporated herein by reference in its entirety.

Briefly, in PCR™, two primer sequences are prepared that are complementary to regions on opposite complementary strands of the marker sequence. An excess of deoxynucleoside triphosphates are added to a reaction mixture along with a DNA polymerase, e.g., Taq polymerase. If the marker sequence is present in a sample, the primers will bind to the marker and the polymerase will cause the primers to be extended along the marker sequence by adding on nucleotides. By raising and lowering the temperature of the reaction mixture, the extended primers will dissociate from the marker to form reaction products, excess primers will bind to the marker and to the reaction products and the process is repeated.

A reverse transcriptase PCR™ amplification procedure may be performed in order to quantify the amount of mRNA amplified. Methods of reverse transcribing RNA into cDNA are well known and described in Sambrook et al., 2001. Alternative methods for reverse transcription utilize thermostable, RNA-dependent DNA polymerases. These methods are described in WO 90/07641 filed Dec. 21, 1990. Polymerase chain reaction methodologies are well known in the art.

Another method for amplification is the ligase chain reaction (“LCR”), disclosed in EP 0 320 308, incorporated herein by reference in its entirety. In LCR, two complementary probe pairs are prepared, and in the presence of the target sequence, each pair will bind to opposite complementary strands of the target such that they abut. In the presence of a ligase, the two probe pairs will link to form a single unit. By temperature cycling, as in PCR™, bound ligated units dissociate from the target and then serve as “target sequences” for ligation of excess probe pairs. U.S. Pat. No. 4,883,750 describes a method similar to LCR for binding probe pairs to a target sequence.

Methods based on ligation of two (or more) oligonucleotides in the presence of nucleic acid having the sequence of the resulting “di-oligonucleotide,” thereby amplifying the di-oligonucleotide, may also be used in the amplification step of the present invention. Wu et al., (1989), incorporated herein by reference in its entirety.

(iii) Southern/Northern Blotting

Blotting techniques are well known to those of skill in the art. Southern blotting involves the use of DNA as a target, whereas Northern blotting involves the use of RNA as a target. Each provide different types of information, although cDNA blotting is analogous, in many aspects, to blotting or RNA species.

Briefly, a probe is used to target a DNA or RNA species that has been immobilized on a suitable matrix, often a filter of nitrocellulose. The different species should be spatially separated to facilitate analysis. This often is accomplished by gel electrophoresis of nucleic acid species followed by “blotting” on to the filter.

Subsequently, the blotted target is incubated with a probe (usually labeled) under conditions that promote denaturation and rehybridization. Because the probe is designed to base pair with the target, the probe will binding a portion of the target sequence under renaturing conditions. Unbound probe is then removed, and detection is accomplished as described above.

(iv) Separation Methods

It normally is desirable, at one stage or another, to separate the amplification product from the template and the excess primer for the purpose of determining whether specific amplification has occurred. In one embodiment, amplification products are separated by agarose, agarose-acrylamide or polyacrylamide gel electrophoresis using standard methods. See Sambrook et al., 2001.

Alternatively, chromatographic techniques may be employed to effect separation. There are many kinds of chromatography which may be used in the present invention: adsorption, partition, ion-exchange and molecular sieve, and many specialized techniques for using them including column, paper, thin-layer and gas chromatography (Freifelder, 1982).

(v) Detection Methods

Products may be visualized in order to confirm amplification of the marker sequences. One typical visualization method involves staining of a gel with ethidium bromide and visualization under UV light. Alternatively, if the amplification products are integrally labeled with radio- or fluorometrically-labeled nucleotides, the amplification products can then be exposed to x-ray film or visualized under the appropriate stimulating spectra, following separation.

In one embodiment, visualization is achieved indirectly. Following separation of amplification products, a labeled nucleic acid probe is brought into contact with the amplified marker sequence. The probe preferably is conjugated to a chromophore but may be radiolabeled. In another embodiment, the probe is conjugated to a binding partner, such as an antibody or biotin, and the other member of the binding pair carries a detectable moiety.

In one embodiment, detection is by a labeled probe. The techniques involved are well known to those of skill in the art and can be found in many standard books on molecular protocols. See Sambrook et al. (2001). For example, chromophore or radiolabel probes or primers identify the target during or following amplification.

One example of the foregoing is described in U.S. Pat. No. 5,279,721, incorporated by reference herein, which discloses an apparatus and method for the automated electrophoresis and transfer of nucleic acids. The apparatus permits electrophoresis and blotting without external manipulation of the gel and is ideally suited to carrying out methods according to the present invention.

In addition, the amplification products described above may be subjected to sequence analysis to identify specific kinds of variations using standard sequence analysis techniques. Within certain methods, exhaustive analysis of genes is carried out by sequence analysis using primer sets designed for optimal sequencing (Pignon et al, 1994). The present invention provides methods by which any or all of these types of analyses may be used. Using the sequences disclosed herein, oligonucleotide primers may be designed to permit the amplification of sequences throughout the CG18190, Jheh 1, CG7228, lama, disco, CG6664, Casein kinase II β subunit, CG9171, GstE1, cAMP-dependent protein kinase R2, CG15161, MESR3, Meics, Atpalpha, Calx, Rlip, nompC, H15, Lam, Glu-RIIA, Glu-RIIB, Ork1, hyperkinetic and shaker genes that may then be analyzed by direct sequencing.

(vi) Kit Components

All the essential materials and reagents required for detecting variation in gene structure or expression may be assembled together in a kit. This generally will comprise preselected primers and probes. Also included may be enzymes suitable for amplifying nucleic acids including various polymerases (RT, Taq, Sequenase™ etc.), deoxynucleotides and buffers to provide the necessary reaction mixture for amplification. Such kits also generally will comprise, in suitable means, distinct containers for each individual reagent and enzyme as well as for each primer or probe.

2. Immunological Diagnosis

Antibodies (discussed above) of the present invention can be used in detecting alterations in the expression level of sleep-related gene products. In addition, immunologic assays may be able to detect changes in primary or secondary structure of proteins as well. ELISAs and Western blotting are the most common forms of immunologic detection.

In one example, antibodies are immobilized onto a selected surface, preferably a surface exhibiting a protein affinity such as the wells of a polystyrene microtiter plate. After washing to remove incompletely adsorbed material, it is desirable to bind or coat the assay plate wells with a non-specific protein that is known to be antigenically neutral with regard to the test antisera such as bovine serum albumin (BSA), casein or solutions of powdered milk. This allows for blocking of non-specific adsorption sites on the immobilizing surface and thus reduces the background caused by non-specific binding of antigen onto the surface.

After binding of antibody to the well, coating with a non-reactive material to reduce background, and washing to remove unbound material, the immobilizing surface is contacted with the sample to be tested in a manner conducive to immune complex (antigen/antibody) formation. Following formation of specific immunocomplexes between the test sample and the bound antibody, and subsequent washing, the occurrence and even amount of immunocomplex formation may be determined by subjecting same to a second antibody having specificity for the same target, but that differs in binding specificity from the first antibody. Appropriate conditions preferably include diluting the sample with diluents such as BSA, bovine gamma globulin (BGG) and phosphate buffered saline (PBS)/Tween®. These added agents also tend to assist in the reduction of nonspecific background. The layered antisera is then allowed to incubate for from about 2 to about 4 hr, at temperatures preferably on the order of about 25° to about 27° C. Following incubation, the antisera-contacted surface is washed so as to remove non-immunocomplexed material. A preferred washing procedure includes washing with a solution such as PBS/Tween®, or borate buffer.

To provide a detecting means, the second antibody will preferably have an associated enzyme that will generate a color development upon incubating with an appropriate chromogenic substrate. Thus, for example, one will desire to contact and incubate the second antibody-bound surface with a urease, alkaline phosphatase, glucose oxidase, or (horseradish) peroxidase-conjugated anti-human IgG for a period of time and under conditions which favor the development of immunocomplex formation (e.g., incubation for 2 hr at room temperature in a PBS-containing solution such as PBS/Tween®).

After incubation with the second enzyme-tagged antibody, and subsequent to washing to remove unbound material, the amount of label is quantified by incubation with a chromogenic substrate such as urea and bromocresol purple or 2,2′-azino-di-(3-ethyl-benzthiazoline)-6-sulfonic acid (ABTS) and H₂O₂, in the case of peroxidase as the enzyme label. Quantitation is then achieved by measuring the degree of color generation, e.g., using a visible spectrum spectrophotometer.

The preceding format may be altered by first binding the sample to the assay plate. Then, primary antibody is incubated with the assay plate, followed by detecting of bound primary antibody using a labeled second antibody with specificity for the primary antibody.

The antibody compositions of the present invention will find great use in immunoblot or Western blot analysis. The antibodies may be used as high-affinity primary reagents for the identification of proteins immobilized onto a solid support matrix, such as nitrocellulose, nylon or combinations thereof. In conjunction with immunoprecipitation, followed by gel electrophoresis, these may be used as a single step reagent for use in detecting antigens against which secondary reagents used in the detection of the antigen cause an adverse background. Immunologically-based detection methods for use in conjunction with Western blotting include enzymatically-, radiolabel-, or fluorescently-tagged secondary antibodies are considered to be of particular use in this regard.

3. Treating Sleep Defects by Modulating Gene Expression/Function

The present invention also involves, in other embodiments, methods of reducing the need for sleep, or improving sleep deprivation recovery, in a subject. Such methods include modulating the expression level or activity of one or more gene products encoded by CG18190, Jheh 1, CG7228, lama, disco, CG6664, Casein kinase II β subunit, CG9171, GstE1, cAMP-dependent protein kinase R2 , CG15161, MESR3, Meics, Atpalpha, Calx, Rlip, nompC, H15, Lam, Glu-RIIA, Glu-RIIB, Ork1, hyperkinetic and shaker. Further embodiments pertain to methods of inhibiting or increasing sleep in a subject that include modulating the expression level or activity of a gene product encoded by a gene selected from the group consisting of CG18190, Jheh 1, CG7228, lama, disco, CG6664, Casein kinase II β subunit, CG9171, GstE1, cAMP-dependent protein kinase R2, CG15161, MESR3, Meics, Atpalpha, Calx, Rlip, nompC, H15, Lam, Glu-RIIA, Glu-RIIB, Ork1, hyperkinetic and shaker.

The lengthy discussion of expression vectors, antisense, ribozymes, siRNA and gene transfer discussed in previous sections is incorporated into this section by reference. Such agents may be used to inhibit the expression of CG18190, Jheh 1, CG7228, lama, disco, CG6664, Casein kinase II β subunit, CG9171, GstE1, cAMP-dependent protein kinase R2, CG15161, MESR3, Meics, Atpalpha, Calx, Rlip, nompC, H15, Lam, Glu-RIIA, Glu-RIIB, Ork1, and shaker. Also useful will be single chain antibodies, and genetic constructs coding therefor, that are directed to these targets. Particularly useful expression vectors are viral vectors such as adenovirus, adeno-associated virus, herpesvirus, vaccinia virus and retrovirus. Also encompassed are liposomally-encapsulated expression vectors. Pharmaceutical agents that modify the activity or expression of sleep-related gene products, for example those identified according to the screening methods disclosed herein, may also be employed. Such agents include toxins specific for various channels that are identified herein as contributing to sleep function, and antibodies designed to bind near the pore of channels.

Those of skill in the art are aware of how to administer therapeutic agents in accordance with the present invention. For example, gene delivery in vivo may rely on viral or non-viral vectors. For viral vectors, one generally will prepare a viral vector stock. Depending on the kind of virus and the titer attainable, one will deliver 1×10⁴, 1×10⁵, 1×10⁶, 1×10⁷, 1×10⁸, 1×10⁹, 1×10¹⁰, 1×10¹¹ or 1×10¹² infectious particles to the patient. Similar figures may be extrapolated for liposomal or other non-viral formulations by comparing relative uptake efficiencies. Formulation as a pharmaceutically acceptable composition is discussed below. Various routes are contemplated, including local and systemic, but targeted provision to the heart is preferred. (See, for example Hammond et al., supra, hereby incorporated by reference in its entirety.)

4. Combined Therapy

In many clinical situations, it is advisable to use a combination of distinct therapies. Thus, it is envisioned that, in addition to the therapies described above, one would also wish to provide to the patient more “traditional” pharmaceutical sleep-modifying therapies. Also envisioned are combinations with pharmaceuticals identified according to the screening methods described herein.

Combinations may be achieved by contacting a subject with a single composition or pharmacological formulation that includes both agents, or by contacting the subject with two distinct compositions or formulations, at the same time, wherein one composition includes the expression construct and the other includes the agent. Alternatively, the sleep therapy of the present invention may precede or follow the other agent treatment by intervals ranging from minutes to weeks. In embodiments where the other agent and expression construct are applied separately to the subject, one would generally ensure that a significant period of time did not expire between the time of each delivery, such that the agent and expression construct would still be able to exert an advantageously combined effect on the subject. In such instances, it is contemplated that one would contact the subject with both modalities within about 12-24 hours of each other and, more preferably, within about 6-12 hours of each other, with a delay time of only about 12 hours being most preferred. In some situations, it may be desirable to extend the time period for treatment significantly, however, where several days (2, 3, 4, 5, 6 or 7) to several weeks (1, 2, 3, 4, 5, 6, 7 or 8) lapse between the respective administrations.

It also is conceivable that more than one administration of either a therapeutic gene, protein or therapeutic agent, or the traditional agent will be desired. Various combinations may be employed, where sleep-modifying therapy of the present invention is “A” and the traditional agent is “B”, as exemplified below:

| | | | | | | | |
|---|---|---|---|---|---|---|---|
| A/B/A | B/A/B | B/B/A | A/A/B | B/A/A | A/B/B | B/B/B/A | B/B/A/B |

| | | | | | | |
|---|---|---|---|---|---|---|
| A/A/B/B | A/B/A/B | A/B/B/A | B/B/A/A | B/A/B/A | B/A/A/B | B/B/B/A |
| A/A/A/B | B/A/A/A | A/B/A/A | A/A/B/A | A/B/B/B | B/A/B/B | B/B/A/B |

Other combinations are contemplated as well. An exemplary list of traditional sleep-related drugs includes various sleep-inducing agents such sedatives and tranquilizers. Particular drugs include 40 Winks, acetaminophen, isomethepentene, dichloralphenazone, diphenhydramine, AllerMax Oral, promethazine, anergan, hydroxyzine, lorazepam, Banophen Oral, Benadryl, atarax, ativan, butabarbital, butisol, bydramine, chlordiazepoxide, clorazepate, Compoz Gel Caps, Compox Nighttime Sleep Aid, flurazepam, dalmane, diazepam, diastat, intensol, dihydrex, Diphen Cough, Diphenacen-50 Injection, Diphenhist, diphenydramine, quazepam, Doral, Dormrin OTC, estazolam, clorazepate, Gen-XENE, Genahist Oral, halcion, haloperidol, triazolam, haldol, haldol deconoate, hydroxyzine, hyrexin-50 Injection, Hyzine-50, librium, luminal, phenobarbital, maximum strength Nytol, mephobarbital, mebaral, Mile Nervine Caplets OTC, pentobarbital, nembutal, nordryl, nordryl oral, Nytol Oral OTC, pentazocine, phenergan, ProSom, quazepam, Restall, Restoril, temazepam, Siladryl Oral OTC, Silphen Couhg, Sleep-eze 3 Oral OTC, Sleepinal, Sleepwell 2-nite OTC, Sominex Oral OTC, Sonata, Talacen, Talwin Compound, Talwin NX, Tranxene, Tusstat Syrup, Twilite Oral OTC, Uni-Bent Cough Syrup, valium, vistacot, vistaril, zalepon, and zolpidem.

5. Formulations and Routes for Administration to Patients

Where clinical applications are contemplated, it will be necessary to prepare pharmaceutical compositions in a form appropriate for the intended application. Generally, this will entail preparing compositions that are essentially free of pyrogens, as well as other impurities that could be harmful to humans or animals.

One will generally desire to employ appropriate salts and buffers to render delivery vectors stable and allow for uptake by target cells. Buffers also will be employed when recombinant cells are introduced into a patient. Aqueous compositions of the present invention comprise an effective amount of the vector to cells, dissolved or dispersed in a pharmaceutically acceptable carrier or aqueous medium. Such compositions also are referred to as inocula. The phrase “pharmaceutically or pharmacologically acceptable” refer to molecular entities and compositions that do not produce adverse, allergic, or other untoward reactions when administered to an animal or a human. As used herein, “pharmaceutically acceptable carrier” includes any and all solvents, dispersion media, coatings, antibacterial and antifungal agents, isotonic and absorption delaying agents and the like. The use of such media and agents for pharmaceutically active substances is well know in the art. Except insofar as any conventional media or agent is incompatible with the vectors or cells of the present invention, its use in therapeutic compositions is contemplated. Supplementary active ingredients also can be incorporated into the compositions.

The active compositions of the present invention may include classic pharmaceutical preparations. Administration of these compositions according to the present invention will be via any common route so long as the target tissue is available via that route. This includes oral and nasal administration, but also includes intradermal, subcutaneous, intramuscular, intraperitoneal, intravascular or intravenous injection.

Solutions of the active compounds as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial an antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

As used herein, “pharmaceutically acceptable carrier” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

For oral administration, the polypeptides of the present invention may be incorporated with excipients and used in the form of non-ingestible mouthwashes and dentifrices. A mouthwash may be prepared incorporating the active ingredient in the required amount in an appropriate solvent, such as a sodium borate solution (Dobell's Solution). Alternatively, the active ingredient may be incorporated into an antiseptic wash containing sodium borate, glycerin and potassium bicarbonate. The active ingredient may also be dispersed in dentifrices, including: gels, pastes, powders and slurries. The active ingredient may be added in a therapeutically effective amount to a paste dentifrice that may include water, binders, abrasives, flavoring agents, foaming agents, and humectants.

The compositions of the present invention may be formulated in a neutral or salt form. Pharmaceutically-acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

Upon formulation, solutions will be administered in a manner compatible with the dosage formulation and in such amount as is therapeutically effective. The formulations are easily administered in a variety of dosage forms such as injectable solutions, drug release capsules and the like. For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

### G. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1: Materials and Methods

Analysis of Sleep in Drosophila.

The inventors monitored fly behavior using visual observation, an ultrasound activity monitoring system, and an automatic infrared system (Drosophila Activity Monitoring System, DAMS; Trikinetics, Waltham, Mass.). The ultrasound method (Shaw et al., 2000) allows a continuous, high-resolution measurement of the behavior of a single fly housed inside an ultrasound standing wave chamber (FIG. 1A). Whenever the fly moves its head, wings, or limbs, a perturbation of the standing wave is produced and is counted as a movement. Although very precise, this method is impractical for evaluating sleep/waking parameters in a large-scale project. The DAMS is instead designed to monitor hundreds or thousands of flies simultaneously. One DAMS monitor contains 32 glass tubes, each housing a single fly and enough food for 1-week recording (FIG. 1B). As each fly moves back and forth in its tube, it interrupts an infrared light beam that bisects the tube. Each crossing is counted as a movement and the number of movement every minute are summed up and expressed as “activity index”. Both the ultrasound and the infrared system had been validated by visual observation and give similar results: flies are mostly active and moving around during the day, while during the night they show long periods of immobility that can last several hours (FIG. 1C).

Behavioral quiescence qualifies as sleep only if it is accompanied by a reversible increase in arousal threshold. Arousal threshold in flies has been measured using vibratory, visual, auditory stimuli (Shaw et al., 2000; Nitz et al., 2002) and, more recently, thermal stimuli (inventors' unpublished results). In all cases it was found that flies that had been behaviorally awake immediately before the stimulus readily responded to low and medium stimulus intensities. By contrast, flies that had been behaviorally quiescent for 5 min or more rarely showed a motor response, although they quickly responded when the stimulus intensity was increased. Thus, sleep can be operatively defined in flies as any period of behavioral quiescence (no counts detected by the DAMS) lasting longer than 5 minutes (FIG. 1D).

Analysis of the Response to Sleep Deprivation in Drosophila.

Sleep deprivation can be performed by gentle tapping on the glass tube whenever the fly stops moving for more than 5 min, or automatically. Currently, in the inventors' laboratory, wakefulness is enforced by placing the DAMS monitors vertically within a framed box able to rotate along its major axis under the control of a motor (FIG. 2A). The box can rotate 180° C. clock-wise or counter-clock-wise (2-3 revolutions/min). At the nadir of each rotation, the monitors are dropped 1 cm. This causes the flies to fall from their current position to the bottom of the tube. This method can effectively sleep deprive thousands of flies simultaneously for one or more days. Wild-type flies sleep longer after being sleep deprived (FIGS. 2B-C). Like in mammals, this sleep rebound occurs mainly immediately after the end of the sleep deprivation period (FIG. 2B), is more pronounced after longer (12-24 hours) than after shorter (6 hours) periods of sleep loss, and the recovered sleep only represents a fraction of what was lost (FIG. 2C). Importantly, there is no increase in sleep duration when female flies are subjected to 12 hours of the same stimulation during the day (when they are normally awake), ruling out aspecific effects (FIG. 2C). In mammals, sleep after sleep deprivation is also qualitatively different, i.e., is richer in slow-wave activity, a well-characterized EEG marker of sleep intensity and sleep pressure, and is less fragmented (i.e., there are fewer periods of brief awakenings during sleep; refs. Borbely and Achermann, 1999; Huber et al., 2000). New evidence from the inventors' laboratory shows that in flies sleep continuity is increased and the number of brief awakenings is reduced after sleep deprivation (Huber et al., 2004; FIG. 2D).

Analysis of the Effects of Sleep Loss on Vigilance in Drosophila.

The inventors have assessed the effects of sleep deprivation on vigilance and memory in wild-type flies using vigilance tests and memory tests. In the vigilance test (FIG. 3), the locomotor response induced by a complex stimulus (visual+acoustic+vibratory) produced by a flap vigorously pushed against the glass tubes where the flies are housed is measured. Wild-type flies, as well as most mutant lines tested so far, respond by moving away from the side where the stimulus is delivered. By doing so, they cross the infrared beam, and the latency to crossing is measured by the DAMS monitor. The inventors only consider periods during which flies are awake and spontaneously patrolling the tubes (flies do not respond to the stimulus when asleep). The inventors calculate the mean latency to crossing the infrared beam from the time point at which the stimulus is delivered. For comparison, one then calculates the mean latency to crossing the infrared beam for a time point 1 minute before the stimulus is delivered. The difference before the 2 mean latencies is taken as an indicator of vigilance. Preliminary data show that this difference is reduced in wild-type flies after 24 hours of sleep deprivation, an indication that vigilance is affected by sleep loss (FIG. 4).

Analysis of the Effects of Sleep Loss on Memory in Drosophila.

The ability of flies to learn and to retain memories can be tested using the heat box system, introduced by Dr. Martin Heisenberg (Wustmann et al., 1996; Wustmann and Heisenberg, 1997; Putz and Heisenberg, 2002). In each heating chamber of this apparatus (FIG. 5), a fly can be conditioned to avoid one side of the chamber if the chamber is heated whenever the fly enters that side; in a subsequent memory test without heat, the fly keeps avoiding the heat-associated side. The procedure has been extensively tested and offers several advantages relative to other methods: 1) it is fast, robust, requires little handling and therefore it is suitable to test a large number of flies; 2) flies are freely moving; 3) statistically significant learning curves can be obtained for individual flies. The inventors' laboratory has recently acquired a heat box system with 16 individual heating chambers. The system was built in Germany by the same people who developed the system in Dr. Heisenberg's laboratory several years ago.

### Example 2: Results

The demonstration that Drosophila sleeps has advanced the knowledge of the phylogeny of sleep, supporting the notion that sleep fulfills at least one fundamental function in many divergent animal species. However, Drosophila can also benefit sleep research by offering a powerful tool for the genetic dissection of sleep, just as it has benefited research on circadian rhythms. The inventors have embarked on a large-scale mutagenesis screening in search for flies that need little sleep and/or do not show a sleep rebound after sleep deprivation. The final goal is to screen as many mutant fly lines as there are fly genes. Over the last 3 years, ˜9000 mutant lines have been screened, many of them carrying a mutation in a single gene (Cirelli, 2003). The mutation was caused either by the insertion of a transposon in the fly genome (insertional mutagenesis; ˜3000 lines screened so far), or by ethyl methanesulfonate (EMS, chemical mutagenesis; ˜6000 lines screened so far). Insertional lines such as those available from public stock centers, e.g., the ˜1000 lines of the Berkeley Drosophila Genome Project primary collection (Spradling et al., 1999) and the ˜2300 lines of the Rorth collection (Rorth et al., 1998) include both loss-of-function mutations and gain-of-function mutations. The first are often due to the insertion of a transposon inside a transcription unit, the latter to gene overexpression following the transposon insertion upstream of the transcription start site. Insertional and chemical mutagenesis offer different advantages. Insertional mutagenesis usually allows rapid identification of the mutated gene by sequencing the flanking sequences from one or both ends of the transposon insertion. Moreover, the mobilization of the inserted element can generate new alleles, and expression patterns can be characterized by lacZ staining of tissues. However, transposons do not insert at random into the genome, but have preferred hot spots (Liao et al., 2000). Chemical mutagenesis with EMS, on the other hand, randomly induces small (point) mutations over the entire genome at a reasonable rate, but the molecular characterization of the gene of interest may be not as straightforward.

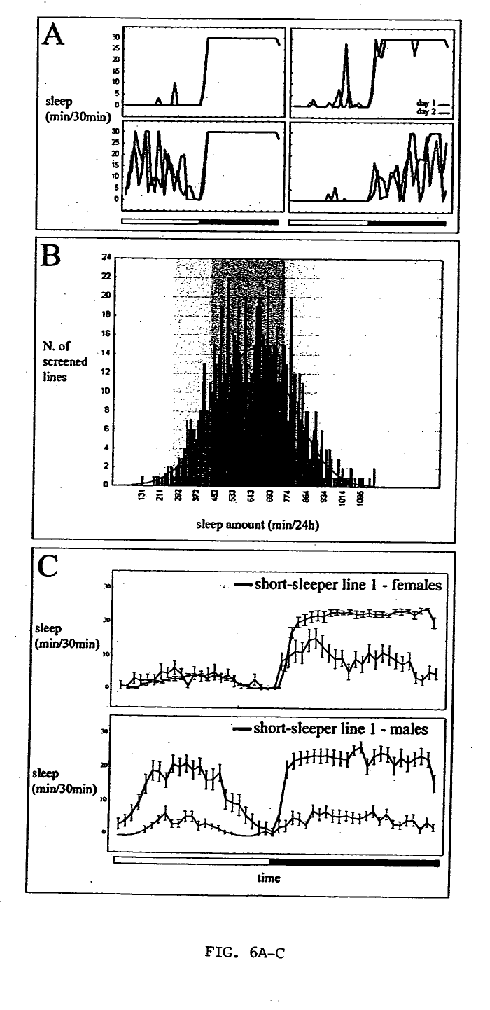

In the current mutagenesis screening, mutant flies were continuously recorded in a DAMS monitor for one week, including 2-3 baseline days, 24 hours of sleep deprivation, and 1-3 days of recovery after sleep deprivation. Ten to sixteen flies (4-7 day old at the beginning of the experiment) were tested for each line. This relatively high number of flies is needed because sleep pattern and sleep amount, although consistent across different days in each individual adult fly, may vary among different flies (FIG. 6A). Interestingly, the analysis of thousand of lines has confirmed a significant difference between male and female flies: while female flies sleep almost exclusively during the night, males show also a long period of sleep in the middle of the day. The daily amount of sleep in the mutant lines tested so far shows a linear distribution, with female flies for most lines sleeping between 400 and 800 min/day, with a mean of ˜600 min, similar to that of wild-type flies (Canton-S female flies=664±137, mean±SD; FIG. 6B). Eighteen lines have so far qualified as “short-sleepers”, i.e., their daily sleep amount is less than 2 standard deviations from the mean of all mutant lines tested so far (<280 min/day in female flies; FIG. 6B). An example of a short sleeper line is shown in FIG. 6C.

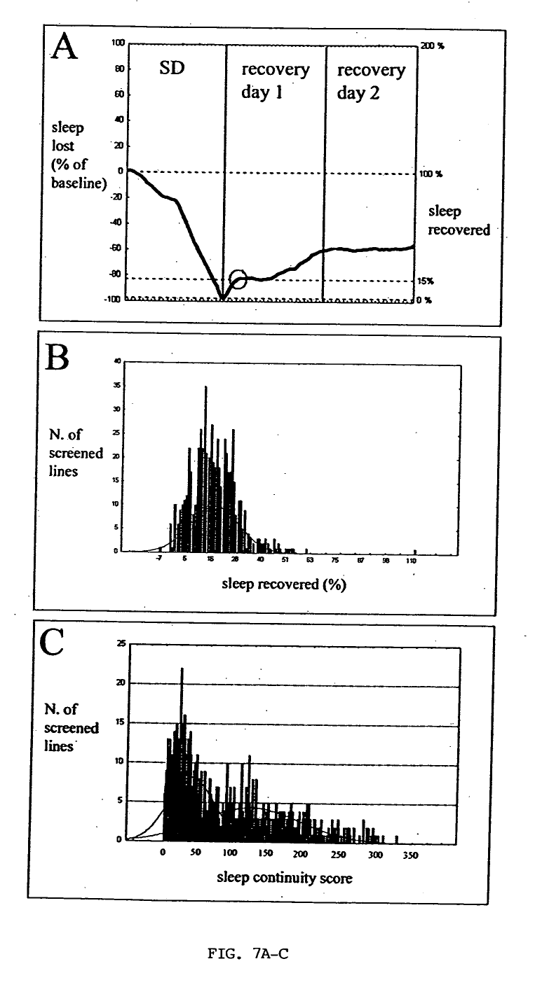

Almost all mutant lines tested so far showed an increase in sleep duration and a decrease in sleep fragmentation after 24 hours of sleep deprivation. As in wild-type flies, the sleep rebound is most pronounced during the first 4-6 hours immediately after the end of the sleep deprivation period, and in most cases does not persist the second day after sleep loss (FIGS. 2B and 7). Similarly to wild-type flies, most mutant lines only recover a small fraction (10-40%) of the sleep lost. So far, the inventors have identified only 5 lines, one of which is also a short sleeper line, which show no sleep rebound after 24 hours of sleep deprivation, suggesting that this phenotype might be even rarer than the short-sleeper phenotype. Since sleep deprivation, as well as chronic sleep restriction, affect vigilance, short sleeper lines and “no-rebound” lines are currently being tested to determine whether their waking performance is normal.

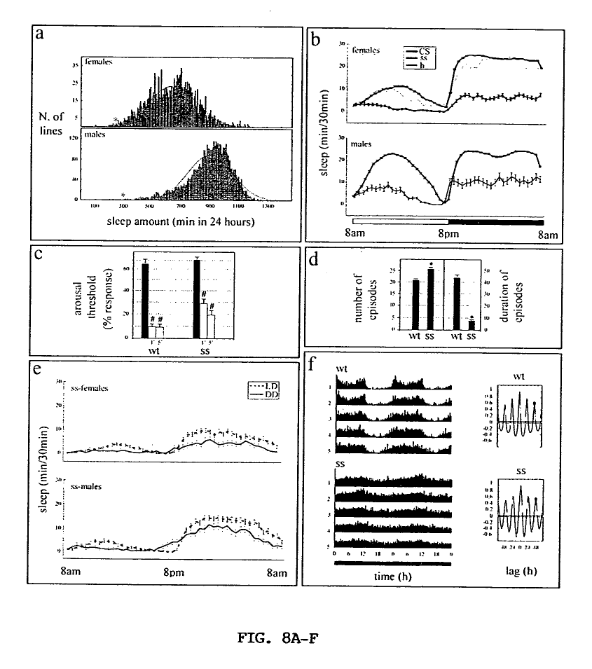

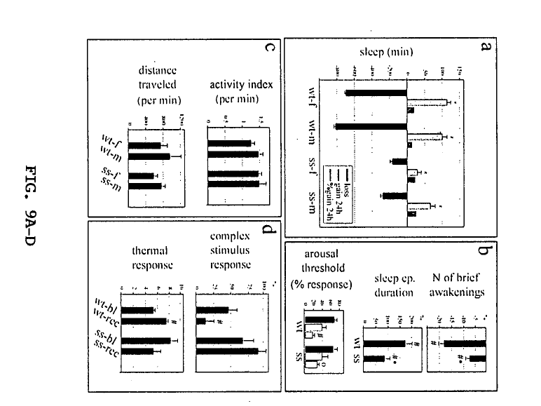

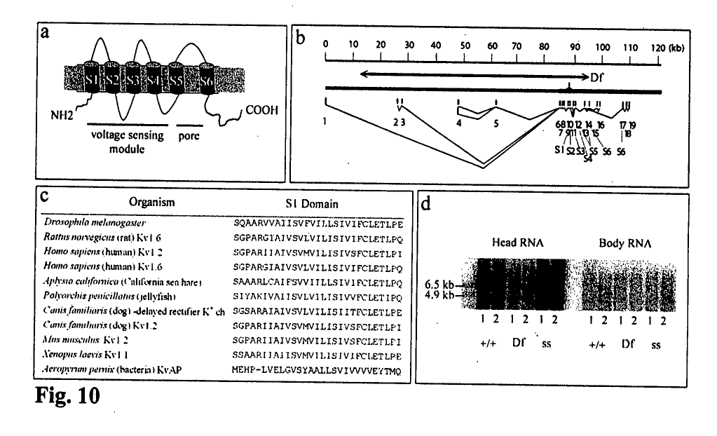

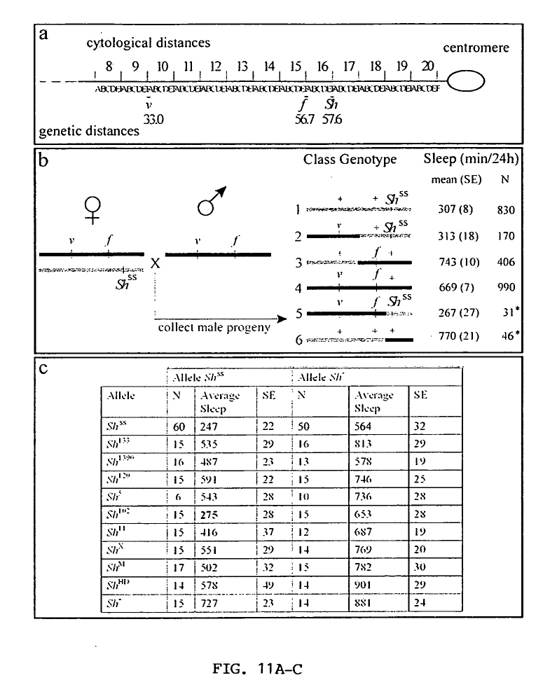

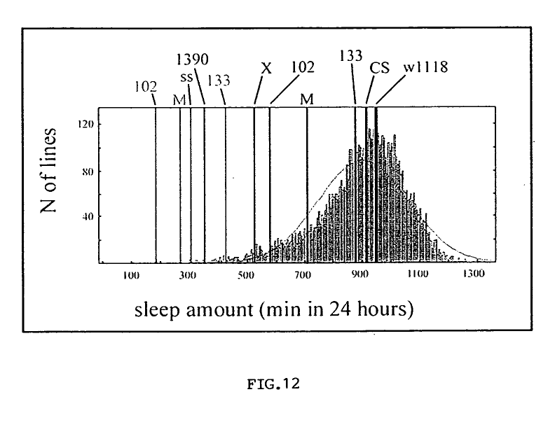

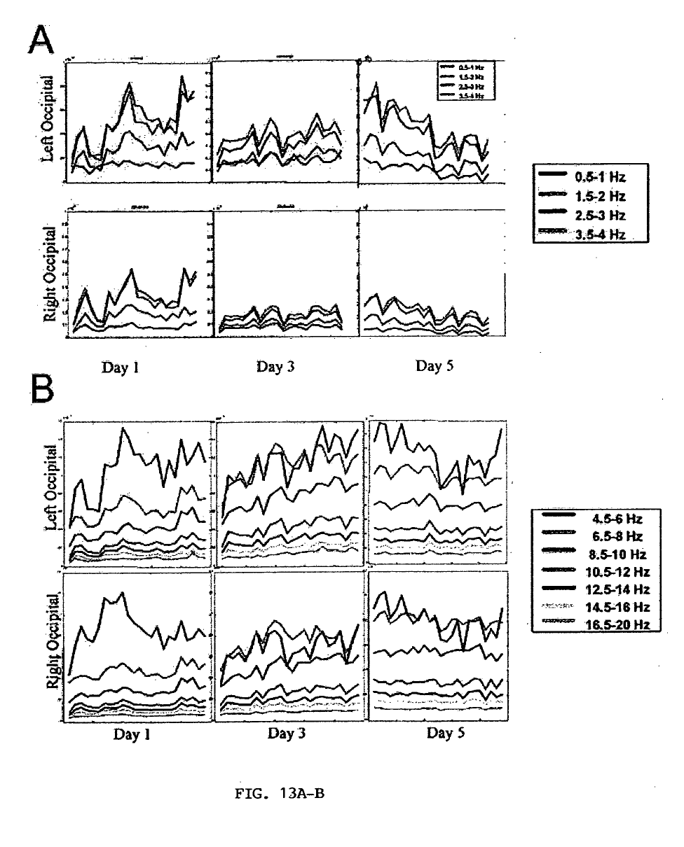

Among the most promising short sleeper lines identified in the mutagenesis screening were EMS lines 1174 and 1179. The inventors have shown (FIGS. 8A-12), the inventors show that 1174 and 1179 flies (called ss flies for short sleepers) sleep only one third of the wild-type amount. Moreover, they show that these flies perform normally in a number of tasks, have preserved sleep homeostasis, but are not impaired by sleep deprivation. The inventors demonstrate in this study that the short sleeper phenotype in 1174 and 1179 flies is due to the same point mutation in a conserved domain of the Shaker gene. Moreover, after crossing out genetic modifiers accumulated over many generations, they also show that other Shaker alleles also become short sleepers. Thus, this study demonstrates that Shaker, which encodes the alpha subunit of a voltage-dependent potassium channel controlling membrane repolarization and transmitter release, may regulate sleep need or efficiency. More recent data from the inventors' laboratory also show that mutations in Hyperkinetic, the gene coding for the beta (regulatory) subunit of the same voltage-potassium channel mutated in lines 1174 and 1179, are also associated with a short sleeper phenotype. The potassium channel coded by Shaker is highly conserved across species, and his mammalian homologues are the potassium channels of the Kv1 family, with Kv 1.2 being the one sharing the highest homology.

A recent report (Liguori et al., 2001) described a patient affected by Morvan's syndrome whose most prominent symptom was a severe and continuous insomnia similar to that reported in patients with fatal familial insomnia (FFI). In the Morvan's patient, the insomnia would temporarily improve after plasma exchange. Antibodies against voltage-dependent K channels, including Kv 1.2, were shown in the patient's CNS by direct immunocytochemistry. Thus, given the established role of Kv channels in the control of neuronal depolarization, the fly mutant data reported here (EMS lines 1174 and 1179), and the Morvan's syndrome report, it is possible that Kv channels play a major role in controlling sleep and waking states. To test this hypothesis, the inventors have custom-designed antibodies capable of binding to a portion of the extracellular pore forming region of the Kv 1.2 channel. Previous studies by Zhou et al. (1998) have shown that antibodies against this region can reduce by 70% the Kv 1.2-mediated current.

Anti-Kv 1.2 is currently administered to rats either via a miniosmotic pump that allows the continuous infusion (1-week) of the antibody in the cerebral cortex of one side or via a bolus injection in the carotid artery following transient opening of the brain blood barrier using mannitol. The goal is to determine whether the antibody infusion can prevent slow wave sleep. Rats are chronically implanted for EEG and EMG recordings. The preliminary control data obtained thus far show that the infusion of rabbit IgGs per se does not affect sleep. Moreover, the histological analysis shows that following the unilateral infusion of anti-Kv 1.2 the antibody can be detected on the extracellular membranes of neurons in the cortical regions targeted by the injection but not elsewhere.

Finally, preliminary experiments show that a unilateral infusion of anti-Kv1.2 is able to reduce the amount of slow waves during sleep (FIGS. 13A-B). The effect can last several days (at least 5 days, as indicated in FIGS. 13A-B), is specific for the site of the injection (in FIGS. 13A-B, low panels restricted to the right side), and is restricted to the frequency band (0.5-4 Hz) typical of slow waves (compare FIGS. 13A and B).

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

H. References

The following references, to the extent that they provide exemplary procedural or other details supplementary to those set forth herein, are specifically incorporated herein by reference.
U.S. Pat. No. 4,196,265
U.S. Pat. No. 4,554,101
U.S. Pat. No. 4,683,195
U.S. Pat. No. 4,683,202
U.S. Pat. No. 4,800,159
U.S. Pat. No. 4,883,750
U.S. Pat. No. 5,279,721
U.S. Pat. No. 5,354,855
U.S. Pat. No. 5,792,453
U.S. Pat. No. 5,994,131
U.S. Pat. No. 6,100,242
U.S. Pat. No. 6,406,921
U.S. Pat. No. 6,457,809
Angel et al., Mol. Cell. Biol., 7:2256, 1987.
Angel et al., Cell, 49:729, 1987b.
Angel et al., Mol. Cell. Biol., 7:2256, 1987a.
Atchison and Perry, Cell, 46:253, 1986.
Atchison and Perry, Cell, 48:121, 1987.
Auluck et al., Science, 295(5556):865-868, 2002.
Baichwal and Sugden, In: Gene Transfer, Kucherlapati (Ed.), NY, Plenum Press, 117-148, 1986.
Banerji et al., Cell, 27(2 Pt 1):299-308, 1981.
Banerji et al., Cell, 33(3):729-740, 1983.
Barany and Merrifield, In: The Peptides, Gross and Meienhofer (Eds.), Academic Press, NY, 1-284, 1979.
Belenky et al., J. Sleep Res., 12(1):1-12, 2003.
Berkhout et al., Cell, 59:273-282, 1989.
Blanar et al., EMBO J., 8:1139, 1989.
Bodine and Ley, EMBO J., 6:2997, 1987.
Borbély and Achermann, J. Biol. Rhythms, 14(6):557-568, 1999.
Boshart et al., Cell, 41:521, 1985.
Bosze et al., EMBO J., 5(7):1615-1623, 1986.
Braddock et al., Cell, 58:269, 1989.
Bulla and Siddiqui, J. Virol., 62:1437, 1986.
Campbell and Villarreal, Mol. Cell. Biol., 8:1993, 1988.
Campere and Tilghman, Genes and Dev., 3:537, 1989.
Campo et al., Nature, 303:77, 1983.
Capaldi et al., Biochem. Biophys. Res. Comm., 74(2):425-433, 1977.
Celander and Haseltine, J. Virology, 61:269, 1987.
Celander et al., J. Virology, 62:1314, 1988.
Chandler et al., Cell, 33:489, 1983.
Chang et al., Hepatology, 14:134A, 1991.
Chang et al., Mol. Cell. Biol., 9:2153, 1989.
Chatterjee et al., Proc. Nat'l. Acad. Sci. USA, 86:9114, 1989.
Chen and Okayama, Mol. Cell Biol., 7(8):2745-2752, 1987.
Choi et al., Cell, 53:519, 1988.
Cirelli C. Bioessays, 25(10):940-949, 2003.
Coffin, In: Virology, Fields et al. (Eds.), Raven Press, NY, 1437-1500, 1990.
Cohen et al., J. Cell. Physiol., 5:75, 1987.
Costa et al., Mol. Cell. Biol., 8:81, 1988.
Couch et al., Am. Rev. Resp. Dis., 88:394-403, 1963.
Coupar et al., Gene, 68:1-10, 1988.
Cripe et al., EMBO J., 6:3745, 1987.
Culotta and Harner, Mol. Cell. Biol., 9:1376, 1989.
Dandolo et al., J. Virology, 47:55-64, 1983.
De Villiers et al., Nature, 312(5991):242-246, 1984.
Deschamps et al., Science, 230:1174-1177, 1985.
Dubensky et al., Proc. Nat'l. Acad. Sci. USA, 81:7529-7533, 1984.
Edbrooke et al., Mol. Cell. Biol., 9:1908, 1989.
Edlund et al., Science, 230:912-916, 1985.
EPO 0 273 085
EPO 320 308
Fechheimer et al., Proc. Nat'l. Acad. Sci. USA, 84:8463-8467, 1987.
Feng and Holland, Nature, 334:6178, 1988.
Ferkol et al., FASEB J., 7:1081-1091, 1993.
Firak and Subramanian, Mol. Cell. Biol., 6:3667, 1986.
Fire et al., Nature, 391(6669):806-811, 1998.
Foecking and Hofstetter, Gene, 45(1):101-105, 1986.
Forster and Symons, Cell, 49(2):211-220, 1987.
Fraley et al., Proc. Nat'l. Acad. Sci. USA, 76:3348-3352, 1979.
Freifelder, In: Physical Biochemistry Applications to Biochemistry and Molecular Biology, 2nd Ed. Wm. Freeman and Co., NY, 1982.
Friedmann, Science, 244:1275-1281, 1989.
Fujita et al., Cell, 49:357, 1987.
Gerlach et al., Nature (London), 328:802-805, 1987.
Ghosh and Bachhawat, In: Liver Diseases, Targeted Diagnosis and Therapy Using Specific Receptors and Ligands, Wu et al. (Eds.), Marcel Dekker, NY, 87-104, 1991.
Ghosh-Choudhury et al., EMBO J., 6:1733-1739, 1987.
Gilles et al., Cell, 33:717, 1983.
Gloss et al., EMBO J., 6:3735, 1987.
Godbout et al., Mol. Cell. Biol., 8:1169, 1988.
Gomez-Foix et al., J. Biol. Chem., 267:25129-25134, 1992.
Goodboum and Maniatis, Proc. Nat'l. Acad. Sci. USA, 85:1447, 1988.
Goodboum et al., Cell, 45:601, 1986.
Gopal, Mol. Cell Biol., 5:1188-1190, 1985.
Graham and Prevec, In: Methods in Molecular Biology: Gene Transfer and Expression Protocol, Murray (Ed.), Humana Press, Clifton, N.J., 7:109-128, 1991.
Graham and Van Der Eb, Virology, 52:456-467, 1973.
Graham et al., J. General Virology, 36:59-74, 1977.
Greene et al., Immunology Today, 10:272, 1989
Grishok et al., Science, 287:2494-2497, 2000.
Grosschedl and Baltimore, Cell, 41:885, 1985.
Grunhaus et al., Seminar in Virology, 200(2):535-546, 1992.
Haslinger and Karin, Proc. Nat'l. Acad. Sci. USA, 82:8572, 1985.
Hauber and Cullen, J. Virology, 62:673, 1988.
Hen et al., Nature, 321:249, 1986.
Hensel et al., Lymphokine Res., 8:347, 1989.
Hermonat and Muzycska, Proc. Nat'l. Acad. Sci. USA, 81:6466-6470, 1984.
Herr and Clarke, Cell, 45:461, 1986.
Hersdorffer et al., DNA Cell Biol., 9:713-723, 1990.
Herz and Gerard, Proc. Nat'l. Acad. Sci. USA, 90:2812-2816, 1993.
Hirochika et al., J. Virol., 61:2599, 1987.
Hirsch et al., Mol. Cell. Biol., 10:1959, 1990.
Holbrook et al., Virology, 157:211, 1987.
Holley et al., Bioassays, 19:218-284, 1997.
Holley et al., Bioassays, 19:218-284, 1997.
Horlick and Benfield, Mol. Cell. Biol., 9:2396, 1989.
Horwich et al. J. Virol., 64:642-650, 1990.
Huang et al., Cell, 27:245, 1981.
Huber et al., Neuroreport., 11(15):3321-3325, 2000.
Huber et al., Sleep 27:628-39, 2004.
Hug et al., Mol. Cell. Biol., 8:3065, 1988.
Hwang et al., Mol. Cell. Biol., 10:585, 1990.
Imagawa et al., Cell, 51:251, 1987.
Imbra and Karin, Nature, 323:555, 1986.
Imler et al., Mol. Cell. Biol., 7:2558, 1987.
Imperiale and Nevins, Mol. Cell. Biol., 4:875, 1984.
Innis et al., PCR Protocols, Academic Press, Inc., San Diego Calif., 1990.
Jakobovits et al., Mol. Cell. Biol., 8:2555, 1988.
Jameel and Siddiqui, Mol. Cell. Biol., 6:710, 1986.
Jaynes et al., Mol. Cell. Biol., 8:62, 1988.
Johnson et al., In: Biotechnology And Pharmacy, Pezzuto et al. (Eds.), Chapman and Hall, NY, 1993.
Johnson et al., Mol. Cell. Biol., 9:3393, 1989.
Jones and Shenk, Cell, 13:181-188, 1978.
Joyce, Nature, 338:217-244, 1989.
Kadesch and Berg, Mol. Cell. Biol., 6:2593, 1986.
Kaneda et al., Science, 243:375-378, 1989.
Karin et al., Mol. Cell. Biol., 7:606, 1987.
Karlsson et al., EMBO J., 5:2377-2385, 1986.
Katinka et al., Cell, 20:393, 1980.
Kato et al, J. Biol. Chem., 266:3361-3364, 1991.
Kawamoto et al., Mol. Cell. Biol., 8:267, 1988.
Ketting et al., Cell, 99:133-141, 1999.
Kiledjian et al., Mol. Cell. Biol., 8:145, 1988.
Kim and Cook, Proc. Nat'l. Acad. Sci. USA, 84(24):8788-8792, 1987.
Klamut et al., Mol. Cell. Biol., 10:193, 1990.
Klein et al., Nature, 327:70-73, 1987.
Koch et al., Mol. Cell. Biol., 9:303, 1989.
Kriegler and Botchan, In: Eukaryotic Viral Vectors, Gluzman (Ed.), Cold Spring Harbor: Cold Spring Harbor Laboratory, N.Y., 1982.
Kriegler and Botchan, Mol. Cell. Biol., 3:325, 1983.
Kriegler et al., Cell, 38:483, 1984.
Kriegler et al., Cell, 53:45, 1988.
Kuhl et al., Cell, 50:1057, 1987.
Kunz et al., Nucl. Acids Res., 17:1121, 1989.
Kyte and Doolittle, J. Mol. Biol., 57(1):105-32, 1982.
Larsen et al., Proc Nat'l Acad. Sci. USA., 83:8283, 1986.
Laspia et al., Cell, 59:283, 1989.
Latimer et al., Mol. Cell. Biol., 10: 760, 1990.
Le Gal La Salle et al., Science, 259:988-990, 1993.
Lee et al., Nature, 294:228, 1981.
Lee et al., Nucleic Acids Res., 12:4191-206, 1984.
Levinson et al., Nature, 295:79, 1982.
Levrero et al., Gene, 101: 195-202, 1991.
Liao et al., Proc. Acad. Sci. USA, 97(7):3347-51, 2000.
Liguori et al. Brain, 124(Pt 12):2417-26, 2001.
Lin and Avery, Nature, 402:128-129, 1999.
Lin et al., Mol. Cell. Biol., 10:850, 1990.
Lindsley & Zimm, In Genome of Drosophila Melanogaster, Academic Press, 1992.
Littleton and Ganetzky, Neuron., 26(1):35-43, 2000.
Luria et al., EMBO J., 6:3307, 1987.
Lusky and Botchan, Proc. Nat'l Acad. Sci. USA, 83:3609, 1986.
Lusky et al., Mol. Cell. Biol., 3:1108, 1983.
Macejak and Sarnow, Nature, 353:90-94, 1991.
Majors and Varmus, Proc. Nat'l Acad. Sci. USA, 80:5866, 1983.
Mann et al., Cell, 33:153-159, 1983.
Markowitz et al., J. Virol., 62:1120-1124, 1988.
McNeall et al., Gene, 76:81, 1989.
Merrifield, Science, 232(4748):341-347, 1986.
Michel and Westhof, J. Mol. Biol., 216:585-610, 1990.
Miksicek et al., Cell, 46:203, 1986.
Montgomery et al., Proc. Nat'l Acad. Sci. USA, 95:155-2-15507, 1998.
Mordacq and Linzer, Genes and Dev., 3:760, 1989.
Moreau et al., Nucl. Acids Res., 9:6047, 1981.
Muesing et al., Cell, 48:691, 1987.
Ng et al., Nuc. Acids Res., 17:601, 1989.
Nicolas and Rubenstein, In: Vectors: A survey of molecular cloning vectors and their uses, Rodriguez and Denhardt (Eds.), Stoneham: Butterworth, 494-513, 1988.
Nicolau and Sene, Biochim. Biophys. Acta, 721:185-190, 1982.
Nicolau et al., Methods Enzymol., 149:157-176, 1987.
Nitz et al., Curr. Biol., 12(22):1934-1940, 2002.
Ondek et al., EMBO J., 6:1017, 1987.
Ornitz et al., Mol. Cell. Biol., 7:3466, 1987.
Pace-Schott and Hobson, Nat. Rev. Neurosci., 3(8):591-605, 2002.
Palmiter et al., Nature, 300:611, 1982.
Paskind et al., Virology, 67:242-248, 1975.
PCT Appln. WO 84/03564
PCT Appln. WO 90/07641
Pech et al., Mol. Cell. Biol., 9:396, 1989.
Pelletier and Sonenberg, Nature, 334(6180):320-325, 1988.
Perales et al., Proc. Nat'l Acad. Sci. USA, 91:4086-4090, 1994.
Perez-Stable and Constantini, Mol. Cell. Biol., 10:1116, 1990.
Picard and Schaffner, Nature, 307:83, 1984.
Pignon et al., Hum. Mutat., 3:126-132, 1994.
Pinkert et al., Genes and Dev., 1:268, 1987.
Pinto et al., Braz. J. Med. Biol. Res., 35(5):599-604, 2002.
Ponta et al., Proc. Nat'l Acad. Sci. USA, 82:1020, 1985.
Porton et al., Mol. Cell. Biol., 10:1076, 1990.
Potter et al., Proc. Nat'l Acad. Sci. USA, 81:7161-7165, 1984.
Putz and Heisenberg, Learn Mem., 9(5):349-359, 2002.
Queen and Baltimore, Cell, 35:741, 1983.
Quinn et al., Mol. Cell. Biol., 9:4713, 1989.
Racher et al., Biotechnology Techniques, 9:169-174, 1995.
Ragot et al., Nature, 361:647-650, 1993.
Redondo et al., Science, 247:1225, 1990.
Reinhold-Hurek and Shub, Nature, 357:173-176, 1992.
Reisman and Rotter, Mol. Cell. Biol., 9:3571, 1989.
Reiter et al., Genome Res., 11(6):1114-1125, 2001.
Remington's Pharmaceutical Sciences, 15^{th} ed., pages 1035-1038 and 1570-1580, Mack Publishing Company, Easton, Pa., 1980.
Renan, Radiother. Oncol., 19:197-218, 1990.
Resendez Jr. et al., Mol. Cell. Biol., 8:4579, 1988.
Rich et al., Hum. Gene Ther., 4:461-476, 1993.
Ridgeway, In: Vectors: A survey of molecular cloning vectors and their uses, Rodriguez and Denhardt (Eds.), Stonehan:Butterworth, 467-492, 1988.
Ripe et al., Mol. Cell. Biol., 9:2224, 1989.
Rippe et al., Mol. Cell Biol., 10:689-695, 1990.
Rittling et al., Nuc. Acids Res., 17:1619, 1989.
Rorth et al., Development, 125(6):1049-1057, 1998.
Rosen et al., Cell, 41:813, 1988.
Rosenfeld et al., Cell, 68:143-155, 1992.
Rosenfeld et al., Science, 252:431-434, 1991.
Roux et al., Proc. Nat'l Acad. Sci. USA, 86:9079-9083, 1989.
Sakai et al., Genes and Dev., 2:1144, 1988.
Sambrook et al., In: Molecular cloning, Cold Spring Harbor Laboratory Press, Cold Spring Harbor, N.Y., 2001.
Saper et al., Trends Neurosci., 24(12):726-731, 2001.
Sarver et al., Science, 247:1222-1225, 1990.
Satake et al., J. Virology, 62:970, 1988.
Scanlon et al., Proc. Nat'l Acad. Sci. USA, 88:10591-10595, 1991.
Schaffner et al., J. Mol. Biol., 201:81, 1988.
Searle et al., Mol. Cell. Biol., 5:1480, 1985.
Sharp and Marciniak, Cell, 59:229, 1989.
Sharp and Zamore, Science, 287:2431-2433, 2000.
Sharp, Genes Dev., 13:139-141, 1999.
Shaul and Ben-Levy, EMBO J., 6:1913, 1987.
Shaw et al., Science., 287(5459):1834-1837, 2000.
Sherman et al., Mol. Cell. Biol., 9:50, 1989.
Sleigh and Lockett, J. EMBO, 4:3831, 1985.
Spalholz et al., Cell, 42:183, 1985.
Spandau and Lee, J. Virology, 62:427, 1988.
Spandidos and Wilkie, EMBO J., 2:1193, 1983.
Spradling et al., Genetics, 153(1):135-177, 1999.
Stephens and Hentschel, Biochem. J, 248:1, 1987.
Stewart and Young, In: Solid Phase Peptide Synthesis, 2d. ed., Pierce Chemical Co., 1984.
Stratford-Perricaudet and Perricaudet, In: Human Gene Transfer, Eds, Cohen-Haguenauer and Boiron, John Libbey Eurotext, France, 51-61, 1991.
Stratford-Perricaudet et al., Hum. Gene. Ther., 1:241-256, 1990.
Stuart et al., Nature, 317:828, 1985.
Sullivan and Peterlin, Mol. Cell. Biol., 7:3315, 1987.
Swartzendruber and Lehman, J. Cell. Physiology, 85:179, 1975.
Tabara et al., Cell, 99:123-132, 1999.
Takebe et al., Mol. Cell. Biol., 8:466, 1988.
Tam et al., J. Am. Chem. Soc., 105:6442, 1983.
Tavernier et al., Nature, 301:634, 1983.
Taylor and Kingston, Mol. Cell. Biol., 10:165, 1990a.
Taylor and Kingston, Mol. Cell. Biol., 10:176, 1990b.
Taylor et al., J. Biol. Chem., 264:15160, 1989.
Temin, In: Gene Transfer, Kucherlapati (Ed.), NY, Plenum Press, 149-188, 1986.
Thiesen et al., J. Virology, 62:614, 1988.
Top et al., J. Infect. Dis., 124:155-160, 1971.
Treisman, Cell, 42:889, 1985.
Tronche et al., Mol. Biol. Med., 7:173, 1990.
Trudel and Constantini, Genes and Dev. 6:954, 1987.
Tur-Kaspa et al., Mol. Cell Biol., 6:716-718, 1986.
Tyndell et al., Nuc. Acids. Res., 9:6231, 1981.
Van Dongen et al., J. Sleep Res., 12(3): 181-187, 2003.
Vannice and Levinson, J. Virology, 62:1305, 1988.
Varmus et al., Cell, 25:23-36, 1981.
Vasseur et al., Proc Nat'l Acad. Sci. U.S.A., 77:1068, 1980.
Wagner et al., Proc. Nat'l Acad. Sci. USA 87(9):3410-3414, 1990.
Wang and Calame, Cell, 47:241, 1986.
Weber et al., Cell, 36:983, 1984.
Weinberger et al. Mol. Cell. Biol., 8:988, 1984.
Winoto and Baltimore, Cell 59:649, 1989.
Wong et al., Gene, 10:87-94, 1980.
Wu and Wu, Adv. Drug Delivery Rev., 12:159-167, 1993.
Wu and Wu, Biochemistry, 27:887-892, 1988.
Wu and Wu, J. Biol. Chem., 262:4429-4432, 1987.
Wu et al., Proc. Nat'l Acad. Sci. USA, 86(8):2757-2760, 1989.
Wustmann and Heisenberg, Learn Mem., 4(4):328-336, 1997.
Wustmann et al., J. Comp. Physiol., 179(3):429-436, 1996.
Yang et al., Proc. Nat'l Acad. Sci. USA, 87:9568-9572, 1990.
Yutzey et al. Mol. Cell. Biol., 9:1397, 1989.
Zars et al., Science, 288(5466):672-675, 2000.
Zelenin et al., FEBS Lett., 280:94-96, 1991.
Zhou et al., J. Gen. Physiol. 111(4):555-63, 1998.

1. A method of screening for a sleep altering composition comprising:
(a) providing a Drosophila cell;
(b) contacting said cell with a candidate compound; and
(c) measuring the effect of said compound on expression level or activity of a first gene product encoded by a gene selected from the group consisting of CG18190, Jheh 1, CG7228, lama, disco, CG6664, Casein kinase II β subunit, CG9171, GstE1, cAMP-dependent protein kinase R2, CG15161, MESR3, Meics, Atpalpha, Calx, Rlip, nompC, H15, Lam, Glu-RIIA, Glu-RIIB, Ork1, Shaker and Hyperkinetic,
whereby a change in the expression level or activity of said gene product, as compared to the expression level or activity of said gene product in a similar cell not treated with said candidate compound, indicates that said candidate compound is a sleep altering composition.

2. The method of claim 1, wherein said Drosophila cell is a neuronal cell.

3. The method of claim 1, wherein the cell is located in a living fly.

4. The method of claim 1, wherein step (c) comprises measuring said expression level.

5. The method of claim 1, wherein step (c) comprises measuring said activity.

6. The method of claim 1, further comprising measuring the effect of said compound on the expression level or activity of a second gene product from said group.

7. The method of claim 4, wherein measuring expression level comprises measuring mRNA levels for said first gene product.

8. The method of claim 4, wherein measuring expression level comprises measuring mRNA turnover for said first gene product.

9. The method of claim 4, wherein measuring expression level comprises measuring protein levels for said first gene product.

10. The method of claim 7, wherein measuring expression level comprises a technique selected from the group consisting of quantitative RT-PCR or Northern blot.

11. The method of claim 9, wherein measuring expression level comprises a technique selected from the group consisting of ELISA or Western blot.

12. The method of claim 5, wherein measuring activity comprises an assay for enzyme function.

13. The method of claim 5, wherein measuring comprises an assay for binding function.

14. The method of claim 1, wherein said composition promotes sleep.

15. The method of claim 1, wherein said composition inhibits sleep.

16. The method of claim 1, wherein said composition promotes recovery from sleep deprivation.

17. The method of claim 1, wherein said composition reduces the need for sleep.

18. The method of claim 1, further comprising measuring the expression level or activity of said gene product in a similar cell not treated with said candidate compound.

19. The method of claim 1, further comprising treating said cell with a known sleep modulating composition.

20. The method of claim 1, further comprising assessing the effect of said candidate substance on an organism.

21. A method of reducing the need for sleep in a subject comprising modulating the expression level or activity of a gene product encoded by a gene selected from the group consisting of CG18190, Jheh 1, CG7228, lama, disco, CG6664, Casein kinase II β subunit, CG9171, GstE1, cAMP-dependent protein kinase R2, CG15161, MESR3, Meics, Atpalpha, Calx, Rlip, nompC, H15, Lam, Glu-RIIA, Glu-RIIB, Ork1, Shaker and Hyperkinetic.

22. The method of claim 21, wherein the expression level or activity of one or more gene produced encoded by a gene of CG18190 or Jheh1 is increased.

23. The method of claim 22, wherein the expression level or activity is increased by providing the gene product or an agonist small molecule to said subject.

24. The method of claim 23, wherein said gene product or agonist is provided to said subject multiple times over a defined period.

25. The method of claim 21, further comprising providing a stimulant to said subject.

26. The method of claim 21, wherein the expression level or activity of Ork1 is decreased.

27. The method of claim 26, wherein the expression level or activity is decreased by providing an antisense molecule, a ribozyme, an interfering RNA or an antagonist small molecule to said subject.

28. The method of claim 27, wherein said antisense molecule, ribozyme, interfering RNA or antagonist small molecule is provided to said subject multiple times over a defined period.

29. The method of claim 21, wherein said subject suffers from a sleep disorder.

30. The method of claim 21, wherein said subject suffers from environmental sleep deprivation.

31. A method of promoting recovery from sleep loss in a subject comprising modulating the expression level or activity of a gene product encoded by a gene selected from the group consisting of CG18190, Jheh 1, CG7228, lama, disco, CG6664, Casein kinase II β subunit, CG9171, GstE1, cAMP-dependent protein kinase R2, CG15161, MESR3, Meics, Atpalpha, Calx, Rlip, nompC, H15, Lam, Glu-RIIA, Glu-RIIB, Ork1, Shaker and Hyperkinetic.

32. The method of claim 31, wherein the expression level or activity of one or more of CG18190 or Jheh1 is increased.

33. The method of claim 32, wherein the expression level or activity is increased by providing the gene product or an agonist small molecule to said subject.

34. The method of claim 33, wherein said gene product or agonist is provided to said subject multiple times over a defined period.

35. The method of claim 31, further comprising providing a stimulant to said subject.

36. The method of claim 31, wherein the expression level or activity of one or more of Ork1 is decreased.

37. The method of claim 36, wherein the expression level or activity is decreased by providing an antisense molecule, a ribozyme, an interfering RNA or an antagonist small molecule to said subject.

38. The method of claim 37, wherein said antisense molecule, ribozyme, interfering RNA or antagonist small molecule is provided to said subject multiple times over a defined period.

39. The method of claim 31, wherein said subject suffers from a sleep disorder.

40. The method of claim 31, wherein said subject suffers from environmental sleep deprivation.

41. A method of inhibiting sleep in a subject comprising modulating the expression level or activity of a gene product encoded by a gene selected from the group consisting of CG18190, Jheh 1, CG7228, lama, disco, CG6664, Casein kinase II β subunit, CG9171, GstE1, cAMP-dependent protein kinase R2, CG15161, MESR3, Meics, Atpalpha, Calx, Rlip, nompC, H15, Lam, Glu-RIIA, Glu-RIIB, Ork1, Shaker and Hyperkinetic.

42. The method of claim 41, wherein the expression level or activity of one or more of CG18190 or Jheh1 is increased.

43. The method of claim 42, wherein the expression level or activity is increased by providing the gene product or an agonist small molecule to said subject.

44. The method of claim 43, wherein said gene product is provided to said subject multiple times over a defined period.

45. The method of claim 41, further comprising providing a stimulant to said subject.

46. The method of claim 41, wherein the expression level or activity of one or more of Ork1 is decreased.

47. The method of claim 46, wherein the expression level or activity is decreased by providing an antisense molecule, a ribozyme, an interfering RNA or an antagonist small molecule to said subject.

48. The method of claim 47, wherein said gene product is provided to said subject multiple times over a defined period.

49. The method of claim 41, wherein said subject suffers from a sleep disorder.

50. The method of claim 41, wherein said subject suffers from environmental sleep deprivation.

51. A method of increasing sleep in a subject comprising modulating the expression level or activity of a gene product selected from the group consisting of CG18190, Jheh 1, CG7228, lama, disco, CG6664, Casein kinase II β subunit, CG9171, GstE1, cAMP-dependent protein kinase R2, CG15161, MESR3, Meics, Atpalpha, Calx, Rlip, nompC, H15, Lam, Glu-RIIA, Glu-RIIB, Ork1, Shaker and Hyperkinetic.

52. The method of claim 51, wherein the expression level or activity of one or more of Ork1 is increased.

53. The method of claim 52, wherein the expression level or activity is increased by providing the gene product or an agonist small molecule to said subject.

54. The method of claim 53, wherein said gene product is provided to said subject multiple times over a defined period.

55. The method of claim 51, further comprising providing a sedative to said subject.

56. The method of claim 51, wherein the expression level or activity of one or more of CG18190 or Jheh1 is decreased.

57. The method of claim 56, wherein the expression level or activity is decreased by providing an antisense molecule, a ribozyme, an interfering RNA or an antagonist small molecule to said subject.

58. The method of claim 57, wherein said gene product is provided to said subject multiple times over a defined period.

59. The method of claim 51, wherein said subject suffers from a sleep disorder.

60. A method for identifying the basis of a sleep disorder in a subject comprising:
(a) obtaining mRNA from a neuronal cell of said subject; and
(b) measuring the expression level or activity of SEQ ID NO:2, 4, 6, 8, 10, 12, 14, 16, 18, 20, 22, 24, 26, 28-30, 32, 34, 36, 38, 40, 42, 44, 46, 48, 54 and/or 55.
whereby a change in the expression level or activity of a gene product in step (b), as compared to the expression level or activity of said gene product in a similar cell from a normal subject, identifies the basis of said sleep disorder.
